(19) United States
(12) Patent Application Publication
(10) Pub. No.: US 2026/0183413 A1
(43) Pub. Date: Jul. 2, 2026

(54) POLYPEPTIDE CONJUGATION COMPOSITIONS AND METHODS OF USE

(71) Applicant: Catena Biosciences,, Berkeley, CA (US)

(72) Inventors: Marco Lobba, Berkeley, CA (US); Richard Kendall, Berkeley, CA (US); Saurabh Johri, Berkeley, CA (US); Maxwell Nguyen, Berkeley, CA (US); Devin Trinter, Berkeley, CA (US)

(21) Appl. No.: 19/562,554

(22) Filed: Mar. 10, 2026

Related U.S. Application Data
(63) Continuation of application No. PCT/US2024/046215, filed on Sep. 11, 2024.
(60) Provisional application No. 63/581,936, filed on Sep. 11, 2023.
(60) Provisional application No. 63/591,072, filed on Oct. 17, 2023.
(60) Provisional application No. 63/607,467, filed on Dec. 7, 2023.
(60) Provisional application No. 63/631,357, filed on Apr. 8, 2024.

(51) Int. Cl. A61K 47/68

(52) U.S. Cl. CPC A61K 47/6851; A61K 47/68031; A61K 47/68037; A61K 47/6809

(57) ABSTRACT

Provided herein are conjugates for chemoselective modification of at least two polypeptides to at least two payloads.

### CROSS-REFERENCE

This application is a continuation of PCT/US2024/046215, filed on Sep. 11, 2024, which claims the benefit of and priority to U.S. Provisional Patent Application No. 63/581,936, filed Sep. 11, 2023, U.S. Provisional Patent Application No. 63/591,072, filed Oct. 17, 2023, U.S. Provisional Patent Application No. 63/607,467, filed Dec. 7, 2023, and U.S. Provisional Patent Application No. 63/631,357, filed Apr. 8, 2024, the disclosures of which are hereby incorporated by reference in their entirety for all purposes.

### SEQUENCE LISTING

The instant application contains a Sequence Listing which has been submitted via Patent Center and is hereby incorporated by reference in its entirety. Said XML copy, created on Mar. 10, 2026, is named CEB-100USWOC1_SL.xml, and is 107,188 bytes in size.

### BACKGROUND

Coupling biomolecules (e.g., polypeptides such as antibodies) to payloads to generate conjugates, while preserving the function of the biomolecule, has long been a goal of chemical biology and biopharmaceutical research. Examples of conjugates include protein-peptide conjugates for vaccine development, antibody-drug conjugates, and antibody-protein conjugates for immunotherapies.

While many techniques have been developed to allow for attachment of payloads to polypeptides, it has been challenging to develop methods for attaching payloads in a site-specific manner to any position on a polypeptide's surface. There is a need for improved conjugation procedures that can modify a polypeptide or biomolecule in a simple yet site specific manner.

### SUMMARY

The present disclosure provides for chemoselective modification of a polypeptide or biomolecule.

In one aspect, disclosed herein is a conjugate of Formula A, Formula B, Formula C, or Formula D:

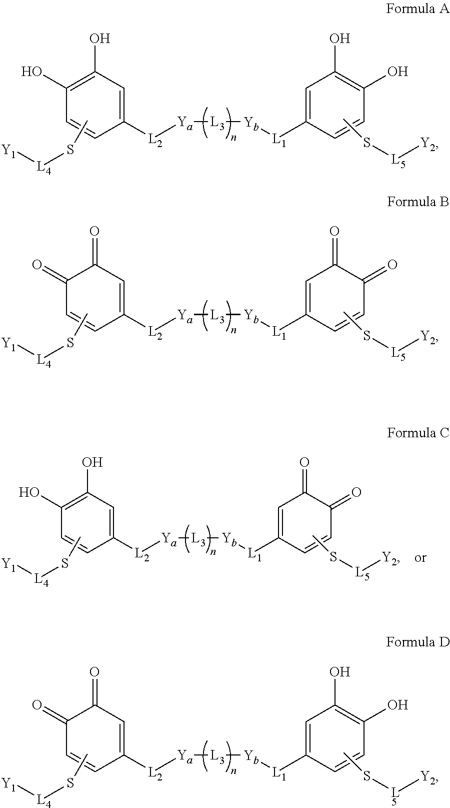

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0;
n is an integer greater than 0;
L₁ is an optional first linker;
L₂ is an optional second linker;
L₃ is an optional third linker;
L₄ is an optional fourth linker;
L₅ is an optional fifth linker;
Y₁ is a first payload; and
Y₂ is a second payload.

In one aspect, disclosed herein is a conjugate of Formula A′, Formula B′, Formula C′, or Formula D′:

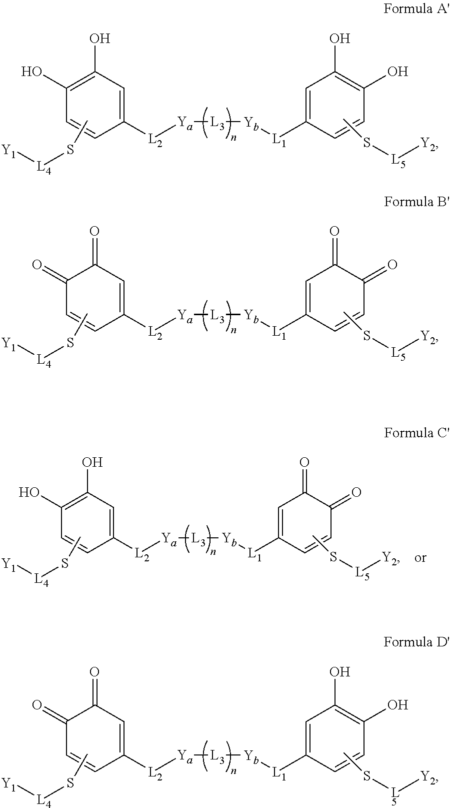

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ comprises a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, wherein Yₐ is linked to “S” via the tyrosine or a portion thereof;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ comprises a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof, wherein Y_{b} is linked to “S” via the tyrosine or a portion thereof;
n is an integer greater than 0;
L₃ is an optional third linker;
L₄ is an optional fourth linker;
L₅ is an optional fifth linker;
Y₁ is a first payload; and
Y₂ is a second payload.

In some embodiments, the tyrosine or a portion thereof is selected from the group consisting of

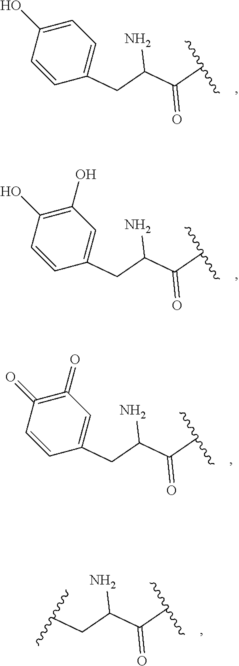

In some embodiments, the first terminal tag comprises at least two amino acids selected from the group consisting of aspartate (D), glutamate (E), arginine (R), and lysine (K).

In some embodiments, the second terminal tag comprises at least two amino acids selected from the group consisting of aspartate (D), glutamate (E), arginine (R), and lysine (K).

In some embodiments, the first terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, the second terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, the first terminal tag comprises SGGY, SGY, SGGGY, ESY, EESY, ESSY, ESSSY, SESY, SGGGY, EESY, SSSY, SNNY, SSNY, EGGY, SESY, or SEGY.

In some embodiments, the second terminal tag comprises EEEY, EEEEY, SSSEY, SSEEY, SSSSY, or SSNNY.

In some embodiments, Yₐ, Y_{b′} or both Yₐ and Y_{b} are glycosylated.

In some embodiments, Yₐ, Y_{b′} or both Yₐ and Y_{b} comprise a non-terminal tyrosine.

In some embodiments, at least one of L₃, L₄, or L₅ is a cleavable linker.

In some embodiments, the cleavable linker is an electrophilically cleavable linker, a nucleophilically cleavable linker, a photocleavable linker, a metal cleavable linker, an electrolytically-cleavable linker, an acid cleavable linker, or a proteolytically cleavable linker.

In some embodiments, the cleavable linker further include a pegylated group, a sugar group, or a modification that increases hydrophilicity.

In some embodiments, the cleavable linker is cleavable under reductive and/or oxidative conditions.

In some embodiments, the cleavable linker is cleavable under acidic conditions.

In some embodiments, the cleavable linker comprises a disulfide bond.

In some embodiments, the cleavable linker is a proteolytically cleavable linker and comprises a protease recognition sequence.

In some embodiments, the protease recognition sequence is recognized by a protease selected from the group comprising a metalloprotease, cathepsin B, and tobacco etch virus (TEV).

In some embodiments, the cleavable linker comprises a dipeptide, tripeptide or tetrapeptide. In some embodiments, the dipeptide is a valine-citrulline (Val-Cit) dipeptide, a valine-lysine dipeptide, a valine-alanine dipeptide. In some embodiments, the tetrapeptide is a glycine-glycine-phenylalanine-glycine (GGFG) tetrapeptide.

In some embodiments, the cleavable linker is selected from the group comprising PABC (p-aminobenzyl alcohol), glucuronide, and MABC (m-aminobenzyl alcohol).

In some embodiments, the cleavable linker is Val-Cit-PABC.

In some embodiments, wherein “Y1-L4-S-” and/or “—S-L5-Y2” is selected from the group consisting of:

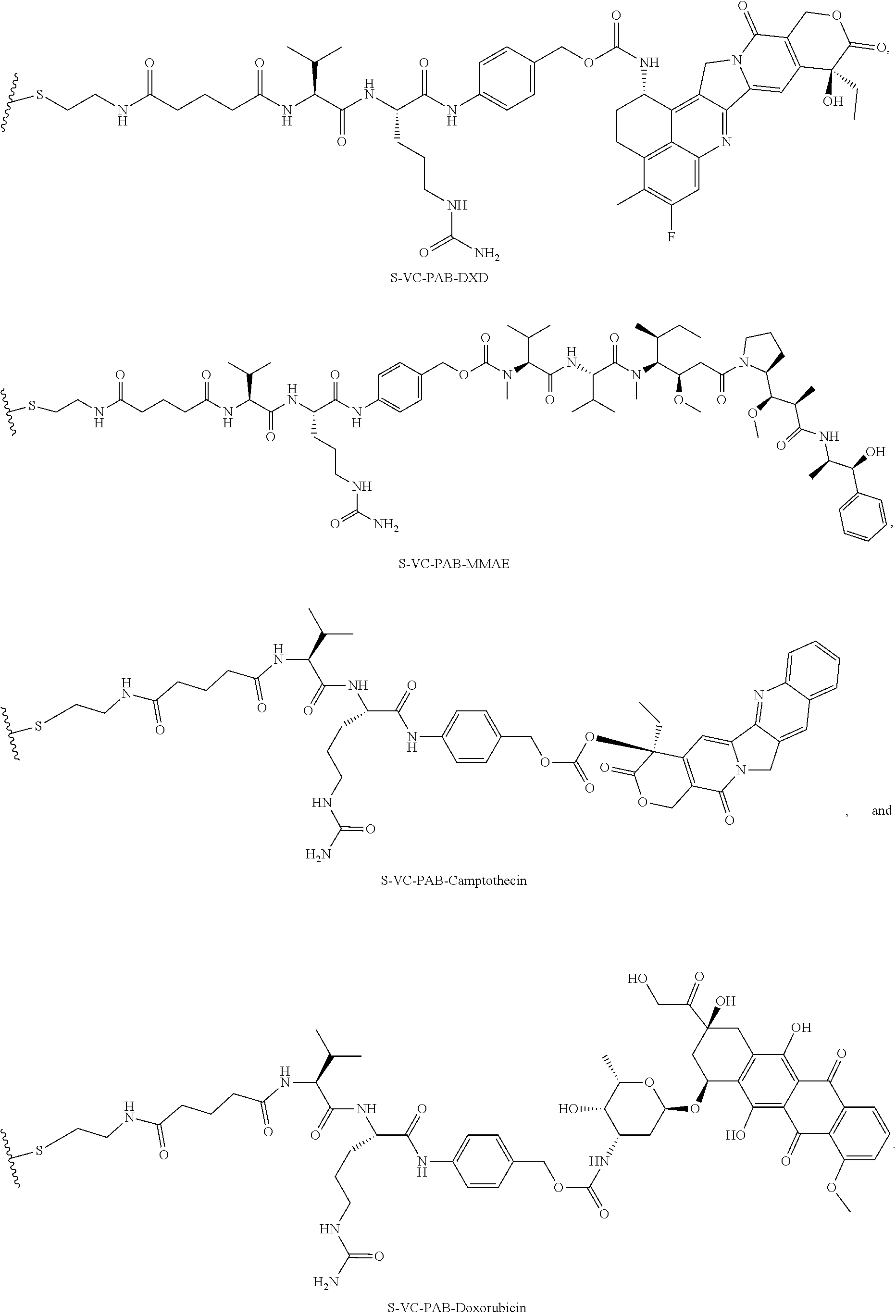

In some embodiments, Yₐ is a heavy chain variable region (VH), a light chain variable region (VL), a heavy chain, a light chain, a constant chain of an antibody or antibody fragment, a peptide, or a cyclic peptide.

In some embodiments, the peptide is a binding peptide.

In some embodiments, Y_{b} is a heavy chain variable region (VH), a light chain variable region (VL), a heavy chain, a light chain, a constant chain of an antibody, antibody fragment, a peptide, or a cyclic peptide. In some embodiments, the peptide is a binding peptide.

In some embodiments, Yₐ and Y_{b} are attached via L₃. In some embodiments, L₃ comprises a peptide sequence, a dimerization and docking domain, a leucine zipper, or knobs-into-holes. In some embodiments, L₃ comprises a peptide bond, a disulfide bond, a maleimide bond, thioether bond, an azide-alkyne cycloaddition, a cystinyl-dopa, or a hydrogen bond. In some embodiments, L₃ is a linker.

In some embodiments, the linker comprises a sequence selected from the group consisting of (GS)ₙ₃, (G2S)ₙ₃, (G3S)ₙ₃, (G4S)ₙ₃, (G)ₙ₃, (GGSGGD)ₙ₃, (GGSGGE)ₙ₃, (GGGSGSGGGGS)ₙ₃, and (GGGGGPGGGGP)ₙ₃ and wherein n3 is an integer from 2 to 20.

In some embodiments, L₃ comprises a terminal tyrosine or a portion thereof.

In some embodiments, n is 1, 2, 3, 4, or 5. In some embodiments, n is 1.

In some embodiments, Y₁ and Y₂ are the same.

In some embodiments, Y₁ and Y₂ are different.

In some embodiments, Y₁, Y₂, or both Y₁ and Y₂ is a small molecule.

In some embodiments, the small molecule is selected from the group consisting of deruxtecan, exatecan, FL 118, irinotecan, topotecan, SN-38, rubitecan, belotecan, lurototecan, gimatecan, diflomotecan, karenitecan, silatecan, namitecan, elomotecan, DRF-1042, delimotecan, NSC606985, chimmitecan, ZBH-1205, auristatin (MMAE, MMAF, MMAG, MMAH) calicheamicin, doxorubicin, taxol and taxol derivatives, maytansinoids (DM1-4), pyrolodiazepines (PBDs), tubulysins, eribulin, anthramycin, duocarmycin, anthracycline, and camptothecin (CPT), including the lactone and carboxylate forms of CPT.

In some embodiments, the small molecule is selected from the group of a topoisomerase inhibitor and a tubulin inhibitor.

In some embodiments, Y₁, Y₂, or both Y₁ and Y₂ comprise a nucleic acid, an immune agonist, a peptide, a cytokine, or a binding domain. In some embodiments, Y₁, Y₂, or both Y₁ and Y₂ comprise a nucleic acid.

In some embodiments, Y₁, Y₂, or both Y₁ and Y₂ comprise an oligonucleotide.

In some embodiments, Y₁, Y₂, or both Y₁ and Y₂ comprises a peptide.

In some embodiments, Y₁, Y₂, or both Y₁ and Y₂ comprises a binding peptide.

In another aspect, provided herein is a conjugate of Formula A-I, Formula B-I, Formula C-I, or Formula D-I:

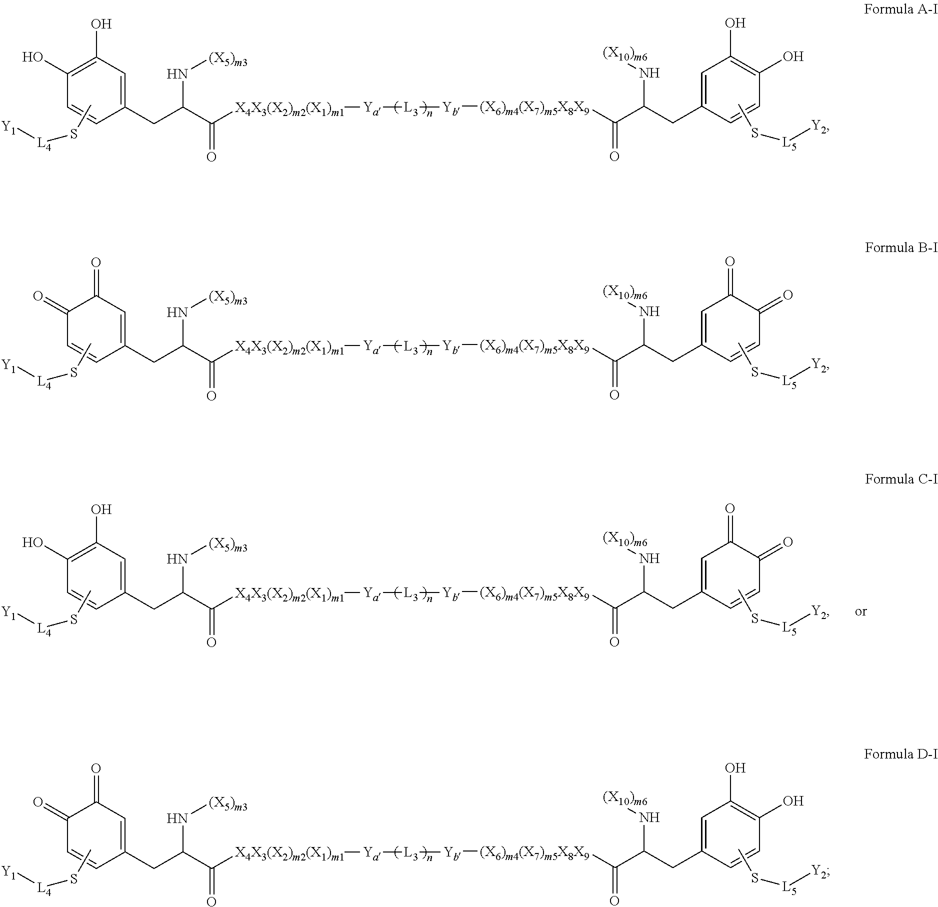

wherein:
Y_{a′} is a first polypeptide,
each of X₁, X₂, X₃, X₄, and X₅ is independently any amino acid, wherein m1, m2, and m3 are each an integer greater than or equal to 0;
Y_{b′} is a second polypeptide,
each of X₆, X₇, X₈, X₉, and X₁₀ is independently any amino acid, wherein m4, m5, and m6 are each an integer greater than or equal to 0, and wherein the —(X₁)ₘ₁(X₂)ₘ₂X₃X₄— moiety is different from the —(X₆)ₘ₁(X₇)ₘ₂X₈ X₉— moiety;
n is an integer greater than 0;
L₃ is an optional third linker;
L₄ is an optional fourth linker
Lₐ is an optional fifth linker
Y₁ is a first payload; and
Y₂ is a second payload.

In some embodiments, the —(X₁)ₘ₁(X₂)ₘ₂X₃X₄— moiety comprises at least two amino acids selected from the group consisting of aspartate (D), glutamate (E), arginine (R), and lysine (K).

In some embodiments, the —(X₆)ₘ₁(X₇)ₘ₂X₈ X₉— moiety comprises at least two amino acids selected from the group consisting of aspartate (D), glutamate (E), arginine (R), and lysine (K).

In some embodiments, the —(X₁)ₘ₁(X₂)ₘ₂X₃X₄— moiety is selected from the group consisting of GGGG, RGGG, RGRG, RRRG, RRRR, EGGG, EGEG, EEEG, EEEE, GGGW, GGWG, RRRW, RRWR, EEEW, EEWE, DDDD, SGG, SG, KKKK, RRKK, RRRK, EDED, EDDD, EEDD, RRKK, KKGG, DDGGEES, ESSS, SSS, SNN, SSN, SEG, SSSE, EGG, SSEE, SES, ESS, ES, SR, SK, SN, ER, EK, EG, SE, SHRK, SARK, SPPE, SSEE, SEEE, and SSSS.

In some embodiments, the —(X₆)ₘ₁(X₇)ₘ₂X₈ X₉— moiety is selected from the group consisting of GGGG, RGGG, RGRG, RRRG, RRRR, EGGG, EGEG, EEEG, EEEE, GGGW, GGWG, RRRW, RRWR, EEEW, EEWE, DDDD, SGG, SG, KKKK, RRKK, RRRK, EDED, EDDD, EEDD, RRKK, KKGG, DDGG, EES, ESSS, SSS, SNN, SSN, SEG, SSSE, EGG, SSEE, SES, ESS, ES, SR, SK, SN, ER, EK, EG, SE, SHRK, SARK, SPPE, SSEE, SEEE, and SSSS.

In some embodiments, the —(X₁)ₘ₁(X₂)ₘ₂X₃X₄— moiety is SGG, SG, SGGG, ES, EES, ESS, ESSS, SES, SGGG, EES, SSS, SNN, SSN, EGG, SES, or SEG.

In some embodiments, the —(X₆)ₘ₁(X₇)ₘ₂X₈ X₉— moiety is EEE, EEEE, SSSE, SSEE, SSSS, or SSNN.

In some embodiments, Y_{a′}, Y_{b′}, or both Y_{a′} and Y_{b′} are glycosylated.

In some embodiments, at least one of L₃, L₄, or L₅ is a cleavable linker. In some embodiments, the cleavable linker is an electrophilically cleavable linker, a nucleophilically cleavable linker, a photocleavable linker, a metal cleavable linker, an electrolytically-cleavable linker, an acid cleavable linker, or a proteolytically cleavable linker.

In some embodiments, the cleavable linker further include a pegylated group, a sugar group, or a modification that increases hydrophilicity. In some embodiments, the cleavable linker is cleavable under reductive and/or oxidative conditions. In some embodiments, the cleavable linker is cleavable under acidic conditions. In some embodiments, the cleavable linker comprises a disulfide bond.

In some embodiments, the cleavable linker is a proteolytically cleavable linker and comprises a protease recognition sequence. In some embodiments, the protease recognition sequence is recognized by a protease selected from the group comprising a metalloprotease, cathepsin B, and tobacco etch virus (TEV).

In some embodiments, the cleavable linker comprises a dipeptide, tripeptide or tetrapeptide. In some embodiments, the dipeptide is a valine-citrulline (Val-Cit) dipeptide, a valine-lysine dipeptide, a valine-alanine dieptide. In some embodiments, the tetrapeptide is a glycine-glycine-phenylalanine-glycine (GGFG) tetrapeptide.

In some embodiments, the cleavable linker is selected from the group comprising PABC (p-aminobenzyl alcohol), glucuronide, and MABC (m-aminobenzyl alcohol),

In some embodiments, Y_{a′} is a heavy chain variable region (VH), a light chain variable region (VL), a heavy chain, a light chain, a constant chain of an antibody or antibody fragment, a peptide, or a cyclic peptide. In some embodiments, the peptide is a binding peptide.

In some embodiments, Y_{b′} is a heavy chain variable region (VH), a light chain variable region (VL), a heavy chain, a light chain, a constant chain of an antibody, antibody fragment, a peptide, or a cyclic peptide. In some embodiments, the peptide is a binding peptide.

In some embodiments, Y_{a′} and Y_{b′} are attached via L3.

In some embodiments, L₃ comprises a peptide sequence, a dimerization and docking domain, a leucine zipper, or knobs-into-holes.

In some embodiments, L₃ comprises a peptide bond, a disulfide bond, a maleimide bond, thioether bond, an azide-alkyne cycloaddition, a cystinyl-dopa, or a hydrogen bond.

In some embodiments, L₃ is a linker. In some embodiments, the linker comprises a sequence selected from the group consisting of (GS)ₙ₃, (G2S)ₙ₃, (G3S)ₙ₃, (G4S)ₙ₃, (G)ₙ₃, (GGSGGD)ₙ₃, (GGSGGE)ₙ₃, (GGGSGSGGGGS)ₙ₃, and (GGGGGPGGGGP)ₙ₃ and wherein n3 is an integer from 2 to 20. In some embodiments, n is 1, 2, 3, 4, or 5. In some embodiments, n is 1.

In some embodiments, Y₁ and Y₂ are the same.

In some embodiments, Y₁ and Y₂ are different.

In some embodiments, Y₁, Y₂, or both Y₁ and Y₂ is a small molecule.

In some embodiments, the small molecule is selected from the group consisting of deruxtecan, exatecan, FL 118, irinotecan, topotecan, SN-38, rubitecan, belotecan, lurototecan, gimatecan, diflomotecan, karenitecan, silatecan, namitecan, elomotecan, DRF-1042, delimotecan, NSC606985, chimmitecan, ZBH-1205, auristatin (MMAE, MMAF, MMAG, MMAH) calicheamicin, doxorubicin, taxol and taxol derivatives, maytansinoids (DM1-4), pyrolodiazepines (PBDs), tubulysins, eribulin, anthramycin, duocarmycin, anthracycline, and camptothecin (CPT), including the lactone and carboxylate forms of CPT.

In some embodiments, the small molecule is selected from the group of a topoisomerase inhibitor and a tubulin inhibitor.

In some embodiments, Y₁ and Y₂, or both Y₁ and Y₂ comprises a nucleic acid, an immune agonist, a peptide, a cytokine, or a binding domain. In some embodiments, Y₁ and Y₂, or both Y₁ and Y₂ comprises a nucleic acid. In some embodiments, Y₁ and Y₂, or both Y₁ and Y₂ comprises an oligonucleotide.

In some embodiments, Y₁ and Y₂, or both Y₁ and Y₂ comprises a peptide. In some embodiments, Y₁ and Y₂, or both Y₁ and Y₂ comprises a binding peptide.

In another aspect, provided herein is a polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, and a second terminal tag different from the first terminal tag, the second terminal tag comprising (X₆)ₘ2X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof.

In some embodiments, the first terminal is on a N-terminus of the polypeptide and the second terminal tag is on the C-terminus of the polypeptide.

In some embodiments, the polypeptide comprises a non-terminal tyrosine.

In some embodiments, the polypeptide is modified to expose the non-terminal tyrosine to be accessible to an enzyme.

In another aspect, provided herein is a composition of a first polypeptide and a second polypeptide, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine or a portion thereof, or X₁X₂X₃, wherein X₁-X₃ is any amino acid provided that at least one amino acid of X₁-X₃ is a tyrosine or a portion thereof, and wherein m1 is an integer greater than or equal to 0, and a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0.

In some embodiments, the first polypeptide and/or the second polypeptide comprise a non-terminal tyrosine.

In some embodiments, the first polypeptide and/or the second polypeptide is modified to expose the non-terminal tyrosine to be accessible to an enzyme.

In some embodiments, the first terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, the second terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, the first terminal tag comprises SGGY, SGY, SGGGY, ESY, EESY, ESSY, ESSSY, SESY, SGGGY, EESY, SSSY, SNNY, SSNY, EGGY, SESY, or SEGY.

In some embodiments, the second terminal tag comprises EEEY, EEEEY, SSEEY, SSSEY, SSSSY, or SSNNY.

In another aspect, provided herein is a method of covalently linking at least two polypeptides to at least two payloads, the method comprising: a) contacting a first polypeptide of the at least two polypeptides and a first payload of the at least two payloads using a first tyrosinase, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, and b) contacting a second polypeptide of the at least two polypeptides and a second payload of the at least two payloads using a second tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof.

In some embodiments, the first tyrosinase, the second tyrosinase, or both the first tyrosinase and the second tyrosinase is Agricus bisporus tyrosinase (abTYR).

In some embodiments, the first tyrosinase, the second tyrosinase, or both comprise a sequence at least 80% identity to any one of SEQ ID NOs: 1-6, 52, or 53.

In some embodiments, the first tyrosinase, the second tyrosinase, or both the first tyrosinase and the second tyrosinase is a Catenase.

In some embodiments, the first tyrosinase and the second tyrosinase is a Catenase.

In some embodiments, the first tyrosinase, the second tyrosinase, or both comprises a sequence at least 90% identity any one of SEQ ID NOs: 2-6.

In some embodiments, the first tyrosinase, the second tyrosinase, or both comprises a sequence at least 90% identity to SEQ ID NO: 2.

In some embodiments, the first tyrosinase is Agricus bisporus tyrosinase (abTYR) and the second tyrosinase is Catenase.

In some embodiments, the first tyrosinase and the second tyrosinase are provided at the same time.

In some embodiments, the first tyrosinase is provided first followed by the second tyrosinase.

In another aspect, provided herein is a method of covalently linking at least two polypeptides to at least two payloads, the method comprising: a) contacting a first polypeptide of the at least two polypeptides and a first payload of the at least two payloads in the presence of a tyrosinase, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, and b) contacting a second polypeptide of the at least two polypeptides and a second payload of the at least two payloads in the presence of the tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof.

In some embodiments, the first polypeptide is contacted with the tyrosinase at a temperature between 0-15° C. In some embodiments, the first polypeptide is contacted with the tyrosinase at a temperature of 4° C.

In some embodiments, the second polypeptide is contacted with the tyrosinase at a temperature between 25-50° C. In some embodiments, the second polypeptide is contacted with the tyrosinase at a temperature of 30° C.

In some embodiments, the first polypeptide is contacted with the tyrosinase for 5-70 min. In some embodiments, the first polypeptide is contacted with the tyrosinase for 45 min. In some embodiments, the first polypeptide is contacted with the tyrosinase for 60 min.

In some embodiments, the second polypeptide is contacted with the tyrosinase for more than 15 min. In some embodiments, the second polypeptide is contacted with the tyrosinase for 60-160 min. In some embodiments, the second polypeptide is contacted with the tyrosinase for 90 min. In some embodiments, the second polypeptide is contacted with the tyrosinase for 120 min.

In some embodiments, the tyrosinase comprises at least 80% identity to any one of SEQ ID NOs: 1-6, 52, or 53.

In another aspect, provided herein is a conjugate of Formula E′, Formula F′, Formula G′, Formula H′, Formula J′, or Formula K′:

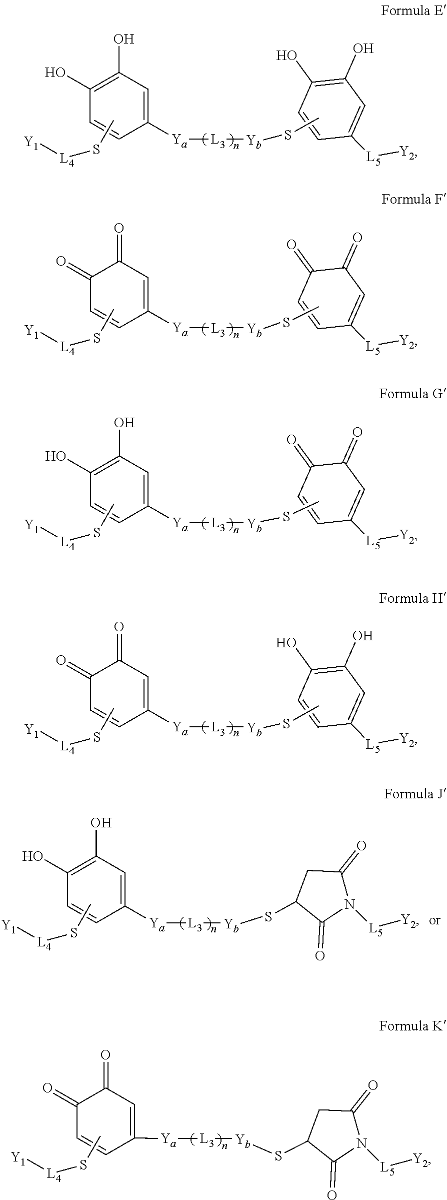

wherein:
Yₐ is a first polypeptide comprising a first terminal tag comprising (X₁)ₘ₁X₂X₃X₄X₅Y, wherein X₁—X₅ is any amino acid provided that no more than two amino acids of X₂, X₃, X₄, and X₅ are aspartate or glutamate and wherein m1 is an integer greater than or equal to 0;
Y_{b} is a second polypeptide;
n is an integer greater than 0;
L₃ is an optional third linker;
L₄ is an optional fourth linker;
L₅ is an optional fifth linker;
Y₁ is a first payload; and
Y₂ is a second payload.

In another aspect, provided herein is a compound of formula L-I:

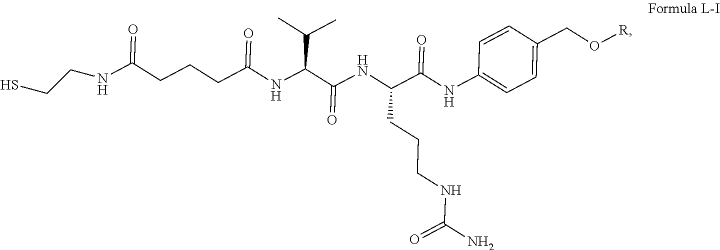

wherein R is a payload.

In some embodiments, the compound is selected from the group consisting of:

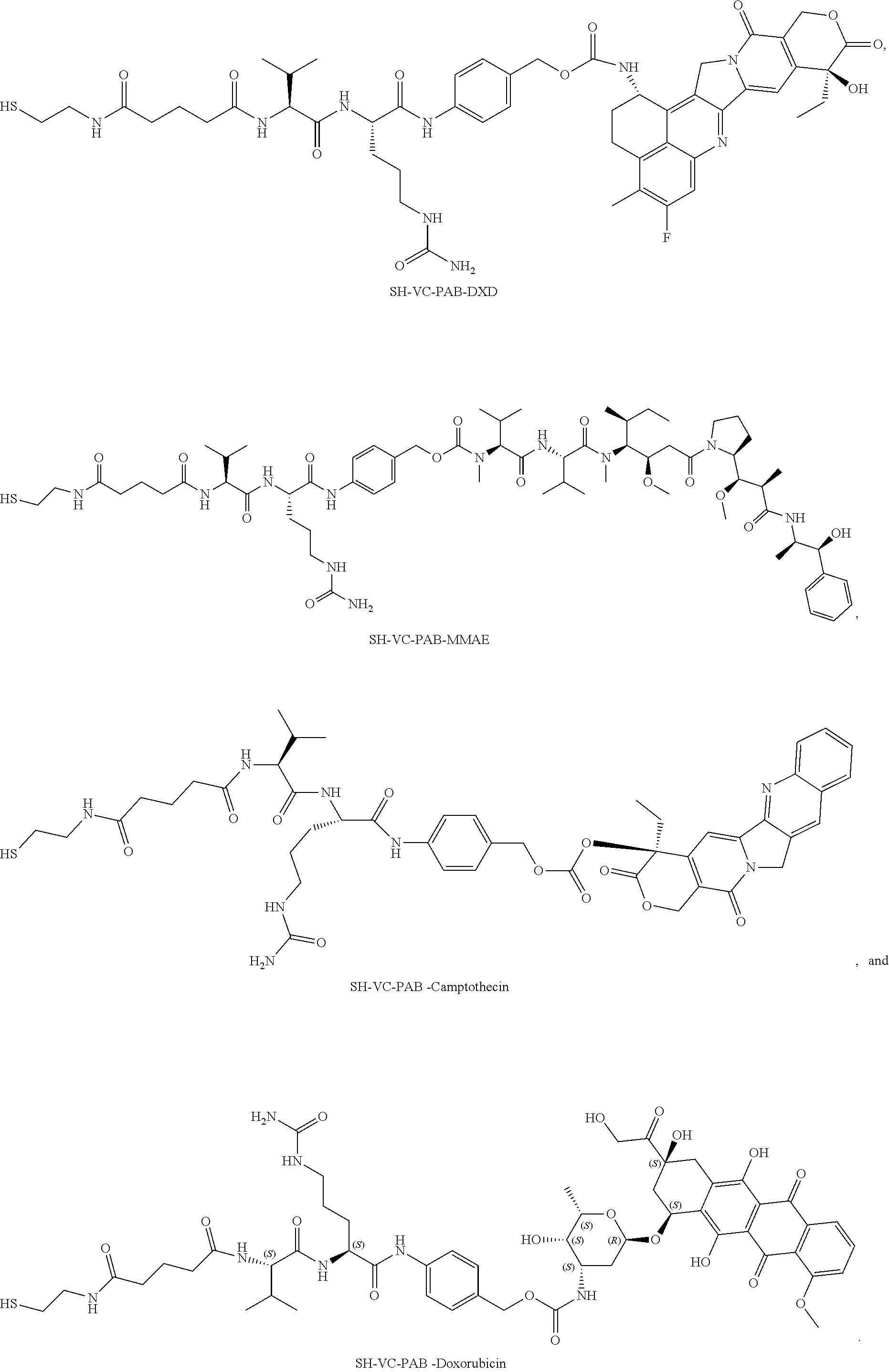

In another aspect, provided herein is a polypeptide-conjugate represented by Formula S1, Formula S2, Formula S3 or Formula S4

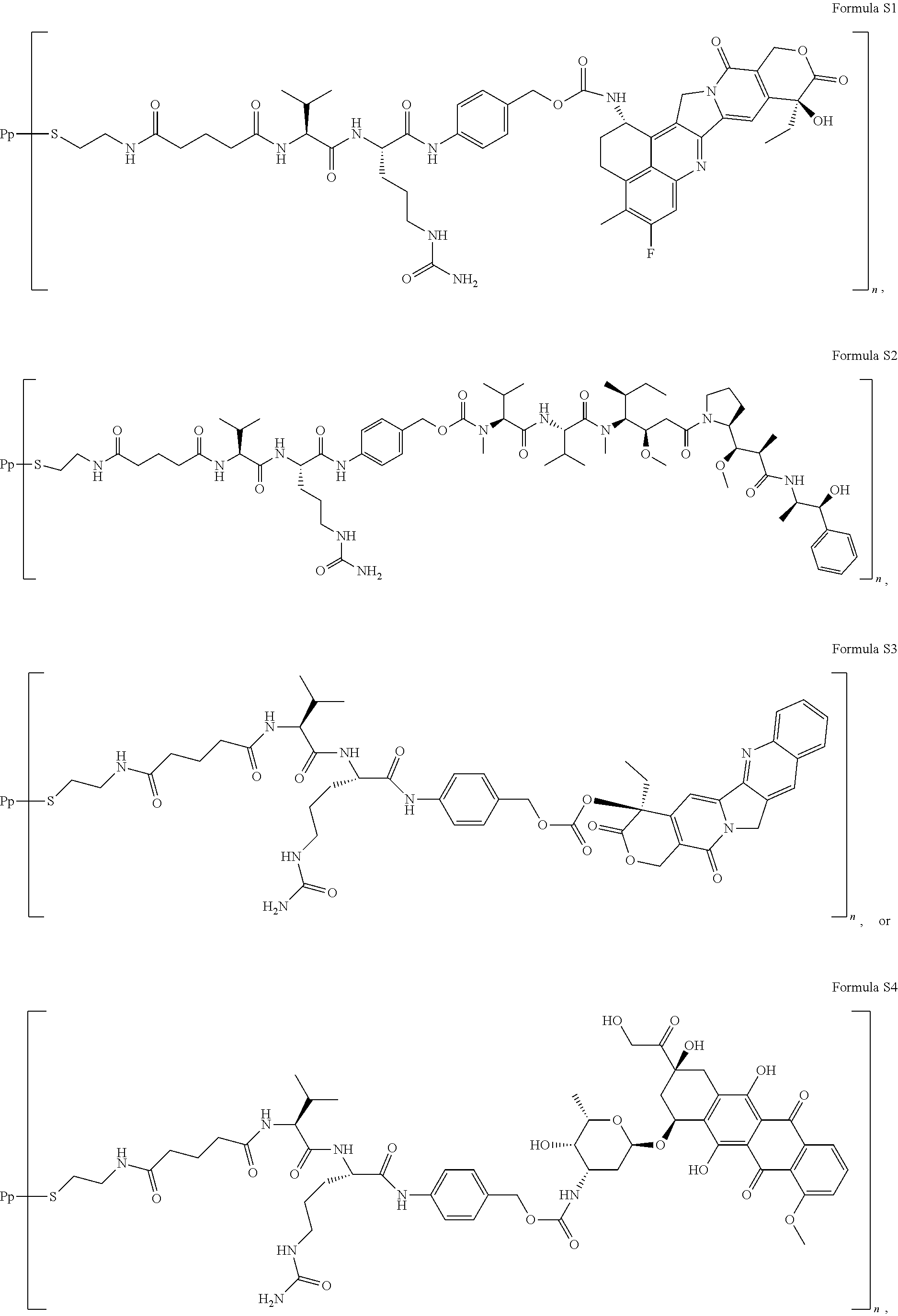

wherein: Pp is a polypeptide and contains an oxidized tyrosine; n is 1, 2, or 3; and wherein the “-S-” of the compound is conjugated to the oxidized tyrosine on the polypeptide.

In some embodiments, the polypeptide is an enzyme, an antibody or a portion thereof, a structural polypeptide, a ligand for a receptor, or a receptor.

In some embodiments, the polypeptide is an antibody or a portion thereof.

In some embodiments, the polypeptide is a heavy chain variable region (VH), a light chain variable region (VL), a heavy chain, a light chain, a constant chain of an antibody or antibody fragment, a peptide, or a cyclic peptide.

### BRIEF DESCRIPTION OF THE DRAWINGS

FIGS. 1A-1B show a summary of characteristics of the compositions and methods for conjugation of a polypeptide or biomolecule to one or more payloads disclosed herein. FIG. 1A shows a bar graph of the selectivity of a method disclosed herein for conjugation of a polypeptide engineered to have a tyrosine tag. FIG. 1B shows the results of a stability assay by showing the protein purification traces of a polypeptide conjugated to a payload by a maleimide bond right after the conjugation reaction and after 7 days in serum at 37° C. compared to a polypeptide conjugated to payload by a CysTyr bond, disclosed in the compositions and methods herein, right after the conjugation reaction and after 7 days in serum at 37° C.

FIG. 2 is an illustration of the different reaction sites accessible through Catenase conjugation, cysteine residues (Site 1), heavy and light chain termini (Sites 2 and 4), and native loop tyrosines (site 3-Y*).

FIG. 3 shows in vitro activity data for Trastuzumab-based ADCs. Panel A of FIG. 3 is the chemical structure of monomethyl auristatin E (MMAE), an exemplary payload, with exemplary linkers used for conjugation to a Trastuzumab-based antibody. Panels B-G of FIG. 3 show the results of in vivo activity data for a Trastuzumab-based antibody drug conjugate (ADC) produced with a CysTyr bond (Panels B-E of FIG. 3) and maleimide bond (Panels F-G of FIG. 3). In vivo activity was tested in HER2 expressing cell lines (Panels B, D, and F of FIG. 3) and HER2-negative cell lines (Panels C, E, and G of FIG. 3).

FIG. 4A shows a Coomassie-stained gel showing intact and reducing conditions for antibodies modified with 2 protein cargoes (1 cargo per heavy chain).

FIG. 4B shows the results of mass spectrometry analysis to observe the percent of modification on native tyrosine residues. Panel A of FIG. 4B show the result of a control heavy chain and Panel B of FIG. 4B shows the result of a heavy chain after a reaction with an exemplary enzyme, a Catenase.

FIG. 5 shows the results of mass spectrometry analysis of the conjugation of multiple payloads to the same antibody. Panel A of FIG. 5 shows the result of a heavy chain control. Panel B of FIG. 5 shows the result of a light chain control. Panel C of FIG. 5 shows the result of modification of an exemplary heavy chain with the observation of one or two additions of an exemplary payload on the exemplary heavy chain. Panel D of FIG. 5 shows the result of modification of an exemplary light chain with the observation of one or two additions of an exemplary payload on the exemplary light chain.

FIG. 6 shows the results of mass spectrometry analysis of an exemplary antibody heavy chain. The top graph is the mass spectra of a control heavy chain. The second graph from the top is the mass spectra of a heavy chain that underwent a conjugation reaction with an exemplary payload, Payload A, at 4° C. The third graph from the top is the mass spectra of the heavy chain from the second graph from the top after a cleanup step. The fourth graph is the mass spectra of the heavy chain from the third graph from the top after a reaction at 37° C. with a second exemplary payload, Payload B.

FIG. 7 shows the results of mass spectrometry analysis of an exemplary antibody light chain. The top graph is the mass spectra of a control light chain. The second graph from the top is the mass spectra of a light chain that underwent a conjugation reaction with an exemplary payload, Payload A, at 4° C. The third graph from the top is the mass spectra of the light chain from the second graph from the top after a cleanup step. The fourth graph is the mass spectra of the light chain from the third graph from the top after a reaction at 37° C. with a second exemplary payload, Payload B.

FIG. 8 shows the results of mass spectrometry analysis of an exemplary antibody. The top graph is the mass spectra of an antibody with its natural sugars after a conjugation with an enzyme, such as a Catenase. The second graph from the top is the mass spectra of an antibody that has been deglycosylated after a conjugation with an enzyme, such as a Catenase, done at 4° C. The third graph from the top is the mass spectra of an exemplary deglycosylated antibody after a conjugation with an enzyme, such as a Catenase, done at room temperature. The fourth graph from the top is the mass spectra of an exemplary antibody that does not have any sugars after a conjugation with an enzyme, such as a Catenase. The fifth graph is the mass spectra of an exemplary deglycosylated antibody after a conjugation with an enzyme, such as a Catenase, done in warm conditions (e.g. room temperature (22° C.) or 37° C.).

FIGS. 9A-9C shows the results of mass spectrometry analysis of an exemplary HER2 antibody. FIG. 9A is the mass spectra of an unmodified HER2 antibody compared to a HER2 antibody that has been conjugated with multiple payloads. FIG. 9B is a close up of the mass spectra of the light chain before and after conjugation with multiple payloads. FIG. 9C is a close up of the mass spectra of the heavy chain before and after conjugation with multiple payloads.

FIGS. 10A-10G show the results of in vitro activity data for HER2 antibody conjugates (circles) compared to DS8201a (T-DXd, triangles) in high HER2 cell lines BT474 (FIG. 10A), SKBR3 (FIG. 10C), and N87 (FIG. 10D), and low HER2 cell line JIMT-1 (FIG. 10B). DS8201a and one of the exemplary HER2 antibody conjugates show high selectivity through lack of inhibition in HER2 negative MDA-MB-468 cells (FIG. 10E). FIG. 10F and FIG. 10G show the results of in vitro activity data for HER2 antibody conjugates (circles) compared to DS8201a (T-DXd, triangles).

FIG. 11 shows the volume of tumor growth in a mice study comparing various doses of an exemplary HER2 antibody conjugated to a topoisomerase inhibitor and a tubulin inhibitor or Ds8201a (T-Dxd) as compared to a control.

FIG. 12A shows the results of mass spectrometry analysis of an exemplary anti-HER2 antibody with a light chain modified to have a terminal tag. The top graph shows the antibody before a reaction with Catenase at 4° C., and the bottom graph shows the antibody after a reaction with Catenase at 4° C.

FIG. 12B shows the results of mass spectrometry analysis of an exemplary anti-HER2 antibody with a heavy chain modified to have a terminal tag. The top graph shows the antibody before a reaction with Catenase at 4° C., and the bottom graph shows the antibody after a reaction with Catenase at 4° C.

FIG. 13A shows the results of mass spectrometry analysis of an exemplary anti-HER2 antibody with a light chain modified to have a terminal tag. The top graph shows the modified antibody before a reaction with Catenase at 30° C., and the bottom graph shows the modified antibody after a reaction with Catenase at 30° C.

FIG. 13B shows the results of mass spectrometry analysis of an exemplary anti-HER2 antibody with a heavy chain and a light chain each modified to have a terminal tag. The top graph shows the modified antibody before a reaction with Catenase at 30° C., and the bottom graph shows the modified antibody after a reaction with Catenase at 30° C.

FIGS. 14A-14C show results of mass spectrometry analysis of an exemplary anti-HER2 antibody modified to have a terminal tag on the heavy chain, a terminal tag on the light chain, and to include the mutations S298G and T299A on the heavy chain before, and after two reactions with Catenase and an exemplary payload, exatecan. FIG. 14A shows the mass spectrum of the modified antibody before a Catenase reaction. FIG. 14B shows the mass spectrum of the modified antibody after a Catenase reaction at 4° C. for 60 min. FIG. 14C shows the mass spectrum of the modified antibody from FIG. 14B after a second Catenase reaction at 30° C. for 90 min.

FIG. 15A-15D show results of mass spectrometry analysis of an exemplary anti-HER2 antibody modified to have a terminal tag on the heavy chain, a terminal tag on the light chian, and to include the mutations S298G T299A on the heavy chain before and after two reactions with Catenase and two exemplary payloads, MMAE and exatecan. FIG. 15A shows the mass spectrum of the modified antibody before a Catenase reaction. FIG. 15B shows the mass spectrum of the modified antibody after a Catenase reaction with MMAE at 4° C. for 45 min. FIG. 15C shows the mass spectrum of the modified antibody from FIG. 15B after a second Catenase reaction with exatecan at 30° C. for 120 min. FIG. 15D shows the mass spectrum of the modified antibody from FIG. 15C after purification.

FIGS. 16A-16D show the percent modification of a light chain or heavy chain with the indicated terminal tag that was conjugated by Catenase to the exemplary payload. FIG. 16A shows the percent modification of a heavy chain to exemplary payload #1 at 4° C. for 60 min. FIG. 16B shows the percent modification of a heavy chain to exemplary payload #2 at 4° C. for 60 min. FIG. 16C shows the percent modification of a light chain to exemplary payload #1 at 30° C. FIG. 16D shows the percent modification of a light chain to exemplary payload #2 at 30° C.

FIG. 17 shows the percent modification of a heavy chain and light chain of a modified trastuzumab to the indicated payloads by Catenase (SEQ ID NO: 2) at a moderate temperature.

FIG. 18 shows the corresponding mass spectra of the heavy chains and light chains of the reactions in FIG. 17.

FIG. 19 shows the percent modification of a heavy chain and light chain of a modified trastuzumab to the indicated payloads by Catenase at a cold low temperature.

FIG. 20 shows the corresponding mass spectra of the heavy chains and light chains of the reactions in FIG. 19.

FIG. 21 shows an NMR spectra of the synthesized SH-VC-PAB-DXD compound.

FIG. 22 shows an NMR spectra of the synthesized SH-VC-PAB-MMAE compound.

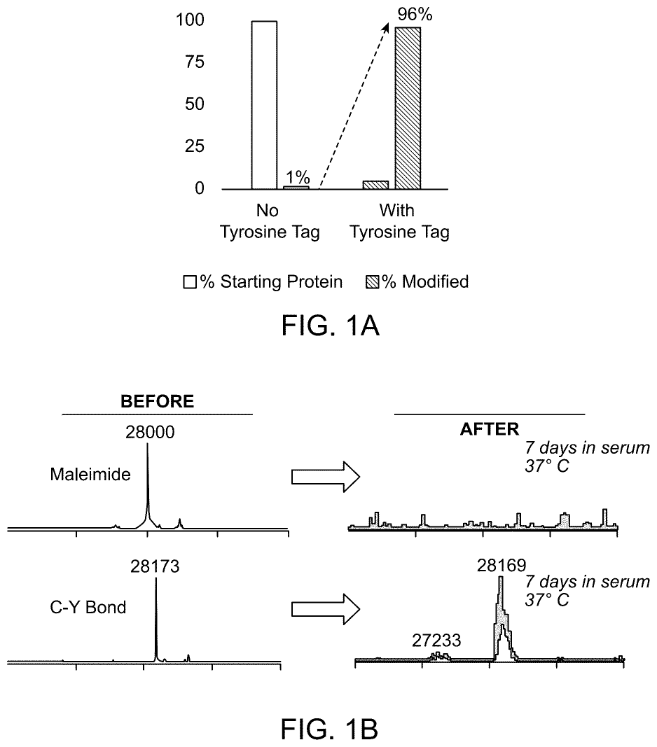

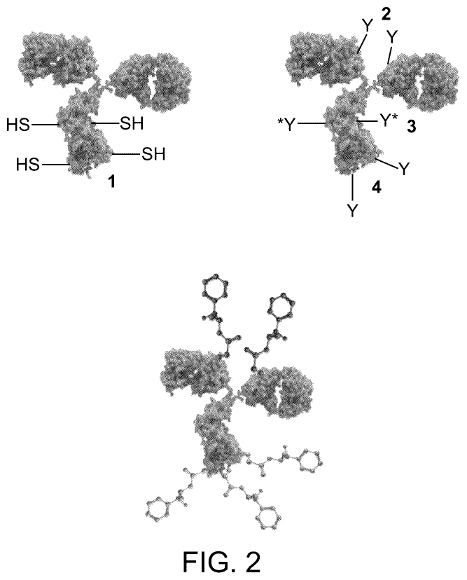

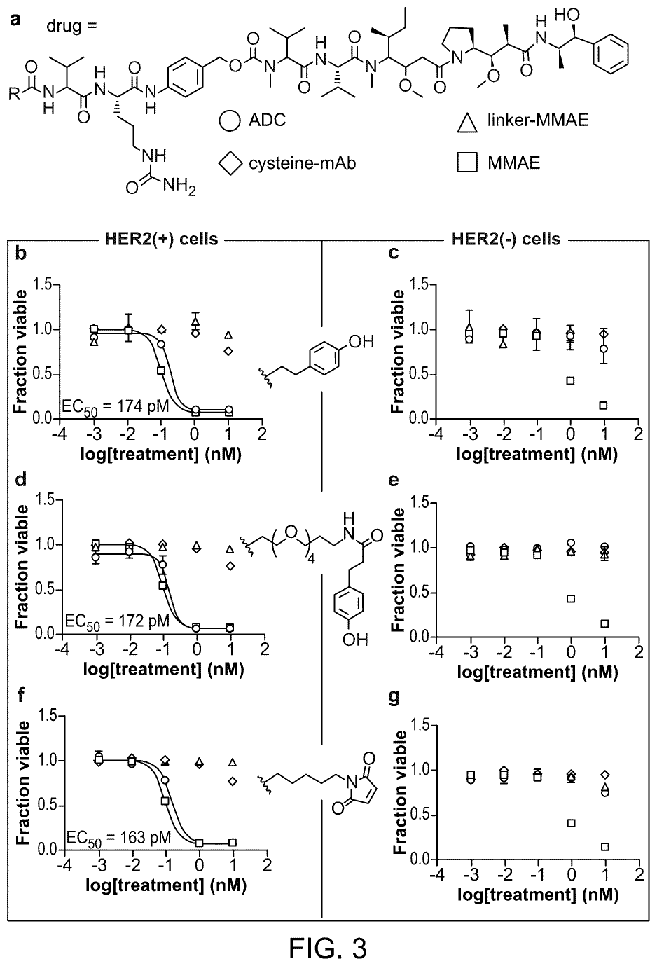

### DETAILED DESCRIPTION

Conjugates such as antibody-drug conjugates are one of the fastest growing drugs for diseases and disorders such as cancer. This approach generally comprises a polypeptide (e.g., an antibody) conjugated to a cytotoxic payload via a chemical linker. Conjugates are complex molecules that require careful attention to various components. Selection of an appropriate target, the polypeptide, the cytotoxic payload, and the manner in which the polypeptide is linked to the payload are key determinants of the safety and efficacy of such conjugates. There is a need for improved conjugation procedures that can modify a polypeptide or biomolecule in a simple yet site specific manner.

Provided herein are compositions and methods for conjugation of a polypeptide or biomolecule to one or more payloads.

### Definitions

Unless otherwise defined, all technical and scientific terms used herein have the same meaning as commonly understood by one of ordinary skill in the art to which this present disclosure pertains. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art.

Throughout this specification and claims, the word “comprise” or variations such as “comprises” or “comprising,” will be understood to imply the inclusion of a stated integer or group of integers but not the exclusion of any other integer or group of integers.

Unless otherwise indicated, and as an example for all sequences described herein under the general format “SEQ ID NO:”, “nucleic acid comprising SEQ ID NO: 1” refers to a nucleic acid, at least a portion of which has either (i) the sequence of SEQ ID NO: 1, or (ii) a sequence complementary to SEQ ID NO: 1. The choice between the two is dictated by the context. For instance, if the nucleic acid is used as a probe, the choice between the two is dictated by the requirement that the probe be complementary to the desired target.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a thiol group” includes a plurality of such thiol groups and reference to “the thiol group” includes reference to one or more thiol groups and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The term “fluorophore” refers to any molecular entity that is capable of absorbing energy of a first wavelength and re-emit energy at a different second wavelength. In certain embodiments, the subject biomolecule includes a fluorophore attached to one end of the biomolecule or at a central position. In some embodiments, the fluorophore may be attached to one end of the biomolecule. The fluorophore attached to the biomolecule need not be a single molecule, but may include multiple molecules.

The fluorophore may be synthetic or biological in nature, as known to those of skill in the art. More generally, any fluorophore can be used that is stable under coupling conditions and that can be sufficiently suppressed when in close proximity to the quencher such that a significant change in the intensity of fluorescence of the fluorophore is detectable in response to target specifically binding the probe. Examples of suitable fluorophores include, but are not limited to Oregon Green 488 dye, rhodamine and rhodamine derivatives, fluorescein isothiocyanate, fluorescein, 6-carboxyfluorescein (6-FAM), coumarin and coumarin derivatives, cyanine and cyanine derivatives, Alexa Fluors, DyLight Fluors, and the like.

In certain embodiments, the biomolecule includes a metal-chelating agent. A “chelate” as used herein in reference to a complex between a metal and a chelating ligand, refers to a combination of a metallic ion bonded to one or more ligands to form a heterocyclic ring structure. Chelate formation through neutralization of the positive charge(s) of the metal ion may be through the formation of ionic, covalent or coordinate covalent bonding. In certain embodiments, the metal chelating agent is includes, but are not limited to, 1,4,7,10-Tetraazacyclododecane-1,4,7,10-tetra acetic acid (also referred to as, DOTA, or tetraxetan).

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. A “polypeptide” encompasses a chemically modified polypeptide. For example, the term “polypeptide” can include a monovalent or divalent radical of i) a chemical structure of a polypeptide or ii) a chemical structure of a polypeptide substituted with a chemical functional group. In some embodiments, a polypeptide is a polypeptide that does not include a terminal tag. In some embodiments, a polypeptide is a small peptide, antibody, or immunoglobulin. The term “fusion protein” or grammatical equivalents thereof is meant to include a protein composed of a plurality of polypeptide components, that while typically separate or unjoined in their native state, typically are joined by their respective amino and carboxyl termini through a peptide linkage to form a single continuous polypeptide. Fusion proteins may be a combination of two, three or even four or more different proteins.

In general, polypeptides may be of any length, e.g., 2 or greater amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally 2 or greater amino acids in length, such as greater than 4 amino acids, greater than about 10 amino acids, greater than about 20 amino acids, usually up to about 50 amino acids. In some embodiments, peptides are between 2 and 30 amino acids in length.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., (1993) Nature 363:446; Desmyter et al., (1996) Nature Struct. Biol. 3:803). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody. [00148]“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

As used herein, the term “Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer.

Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (Chi) of the heavy chain. Fab fragments differ from Fab′ fragments by the addition of a few residues at the carboxyl terminus of the heavy chain Chi domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgGl, IgG2, IgG3, IgG4, IgA, and IgA2. The subclasses can be further divided into types, e.g., IgG2a and IgG2b.

As used herein, “Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen binding fragments.

As used herein, the term “binding” refers to a direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁷ M or greater, e.g., 5×10⁷ M, 10⁸ M, 5×10⁸ M, and greater. “Nonspecific binding” refers to binding with an affinity of less than about 10⁷ M, e.g., binding with an affinity of 10⁶ M, 10⁵ M, 10⁴ M, etc.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of protein as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The term “HER2 positive” or “HER2 high expressing” refers to HER2 overexpression in a cancer. A cancer is classified as “HER2 positive” by an immunohistochemistry (IHC) assay score of 3+ or an immunohistochemistry (IHC) assay score of 2+ with HER2 amplification. Having at least 4 copies of HER2 per tumor cell can also be used to classify a cancer as “HER2 positive” and can be determined using an in situ hybridization (ISH) assay.

The term “HER2 low expressing” refers to low HER2 expression in a cancer. A cancer is classified as “HER2 low expressing” by an immunohistochemistry (IHC) assay score of 0, an immunohistochemistry (IHC) assay score of 1+, or an immunohistochemistry (IHC) assay score of 2+ with no HER2 amplification. “HER2 low expressing” cancer can refer to HER2 negative cancers and HER2 triple negative cancers (estrogen receptor (ER) negative, progesterone receptor (PR) negative, and HER2 negative). Having at most 4 (e.g., at most 3, at most 2, at most 1) copies of HER2 per tumor cell can also be used to classify a cancer as “HER2 low expressing” and can be determined using an in situ hybridization (ISH) assay.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

### Polypeptides of the Disclosure

Described herein, in certain embodiments, are conjugates of Formula A′, Formula B′, Formula C′, or Formula D′:

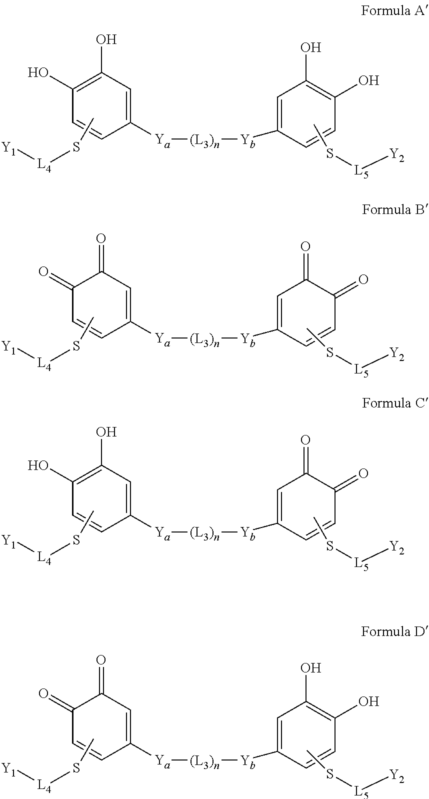

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, wherein Yₐ is linked to “S” through the tyrosine or a portion thereof;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof, wherein Y_{b} is linked to “S” through the tyrosine or a portion thereof;
n is an integer greater than 0;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula (A), Formula (B), Formula (C), or Formula (D):

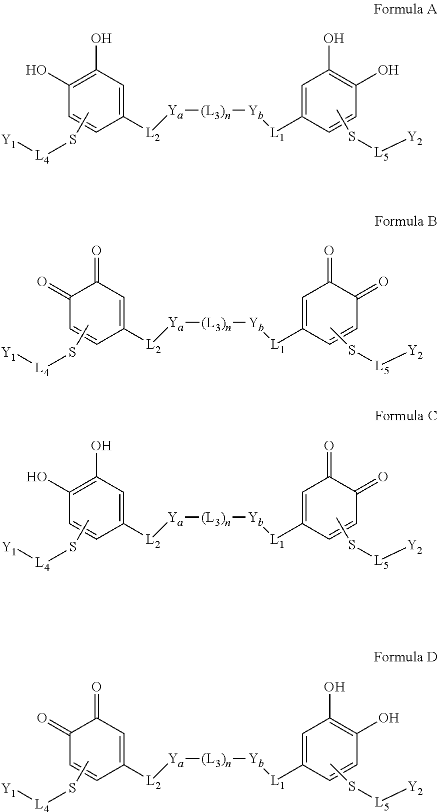

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0;
n is an integer greater than 0;
L₁ is an optional first linker;
L₂ is an optional second linker;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula A″, Formula B″, Formula C″, or Formula D″:

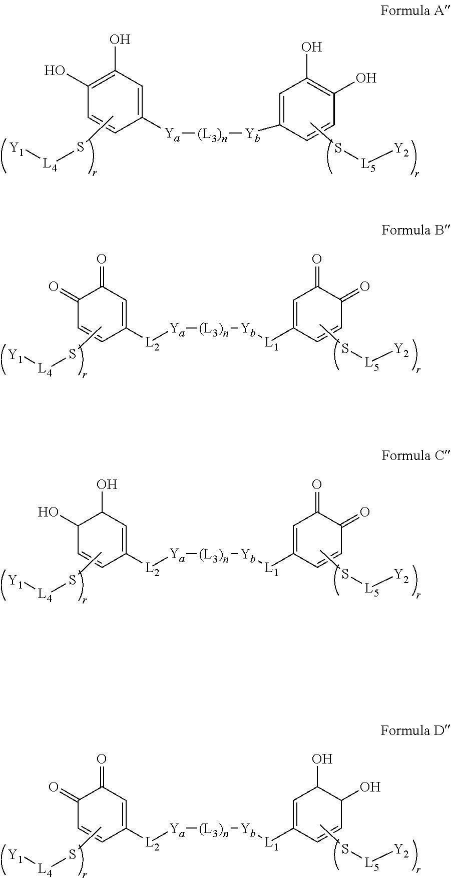

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m₁ is an integer greater than or equal to 0, or X₁X₂X₃, wherein X₁-X₃ is any amino acid provided that at least one amino acid of X₁-X₃ is a tyrosine or a portion thereof, wherein Yₐ is linked to “S” through the tyrosine or a portion thereof;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof, wherein Y_{b} is linked to “S” through the tyrosine or a portion thereof;
r is 1, 2, or 3;
n is an integer greater than 0;
L₃ is an optional third linker;
L₄ is an optional fourth linker;
L₅ is an optional fifth linker;
Y₁ is a first payload; and
   Y₂ is a second payload.

Described herein, in certain embodiments, are conjugates of Formula (A), Formula (B), Formula (C), or Formula (D):

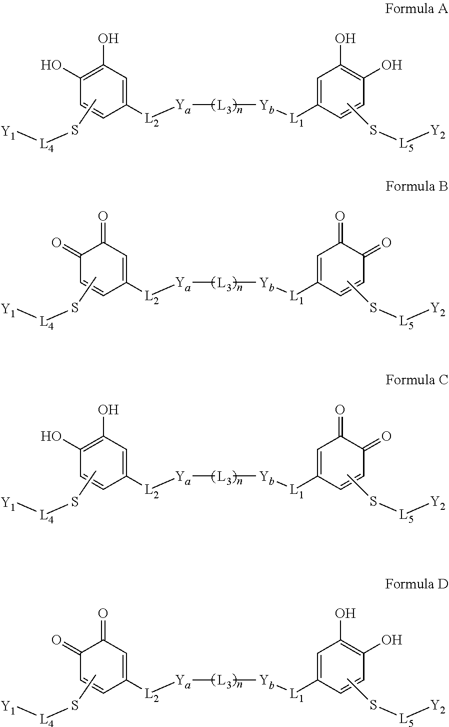

wherein:
Yₐ is a first polypeptide comprising a first terminal tag comprising (X₁)ₘ₁X₂X₃X₄X₅, wherein X₁—X₅ is any amino acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0, or X₁X₂X₃, wherein X₁-X₃ is any amino acid provided that at least one amino acid of X₁-X₃ is a tyrosine;
n is an integer greater than 0;
L₁ is an optional first linker;
L₂ is an optional second linker;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula A′, Formula B′, Formula C′, or Formula D′:

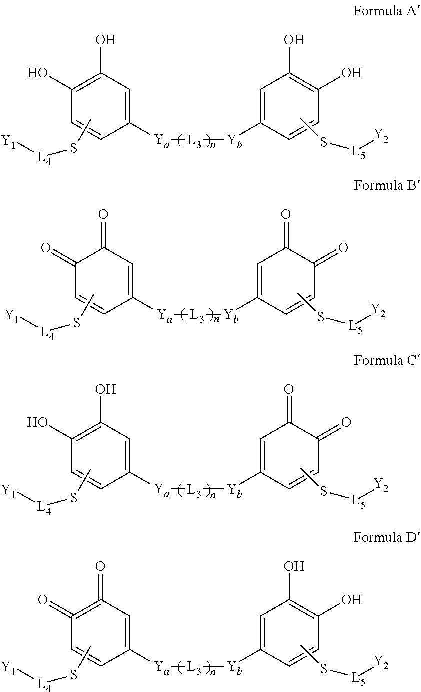

wherein:
Yₐ is a group derived from a first biomolecule comprising a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine, wherein the phenol of the tyrosine is oxidized to form:

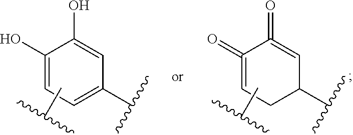

Y_{b} is a second biomolecule comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0, or X₁X₂X₃, wherein X₁-X₃ is any amino acid provided that at least one amino acid of X₁-X₃ is a tyrosine wherein the phenol of the tyrosine is oxidized to form:

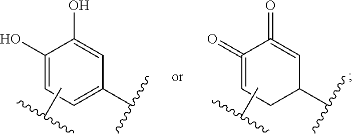

n is an integer greater than 0;
L₃ is an optional first linker;
L₄ is an optional second linker;
L₅ is an optional third linker;
Y₁ is a first payload; and
Y₂ is a second payload

In some embodiments, Yₐ, Y_{b′} or Yₐ and Y_{b} is a nanoparticle, a polymer, a nucleic acid sequence, or an aptamer.

Described herein, in certain embodiments, are conjugates comprising a phenol moiety or a catechol moiety conjugated to one or more payloads comprising a thiol moiety.

Described herein, in certain embodiments, are conjugates of Formula (E), Formula (F), Formula (G) or Formula (H):

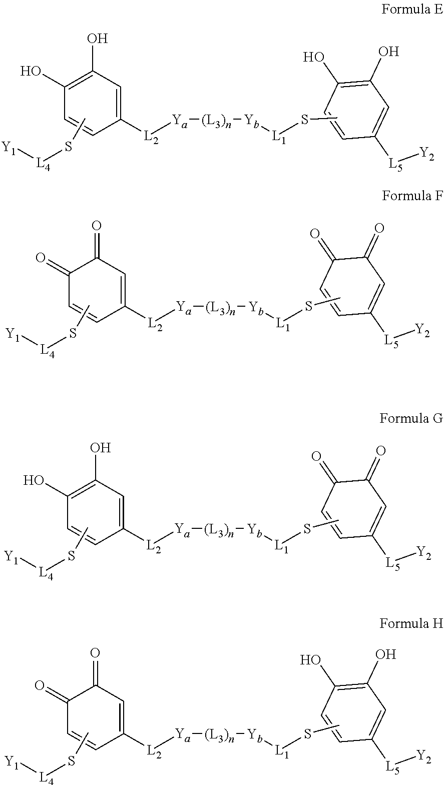

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine, wherein Yₐ is linked to “S” through the tyrosine or a portion thereof,
Y_{b} is a second polypeptide; n is an integer greater than 0;
L₁ is an optional first linker;
L₂ is an optional second linker;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula E′, Formula F′, Formula G′, Formula H′, Formula J′, or Formula K′:

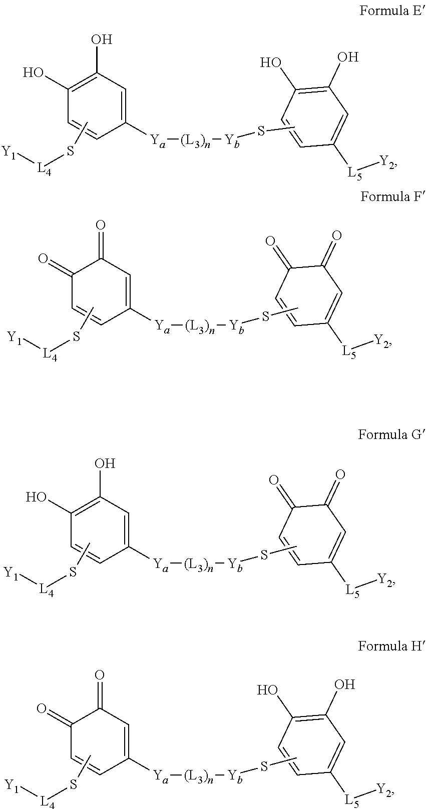

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, wherein Yₐ is linked to “S” through the tyrosine or a portion thereof;
Y_{b} is a second polypeptide;
n is an integer greater than 0;
L₃ is an optional first linker;
L₄ is an optional second linker;
L₅ is an optional third linker in Y₂;
Y₁ is a first payload; and
Y₂ is a group derived from a second payload comprising a phenol, wherein the phenol is linked to “S.”

Described herein, in certain embodiments, are conjugates of Formula E′, Formula F′, Formula G′, Formula H′, Formula J′, or Formula K′:

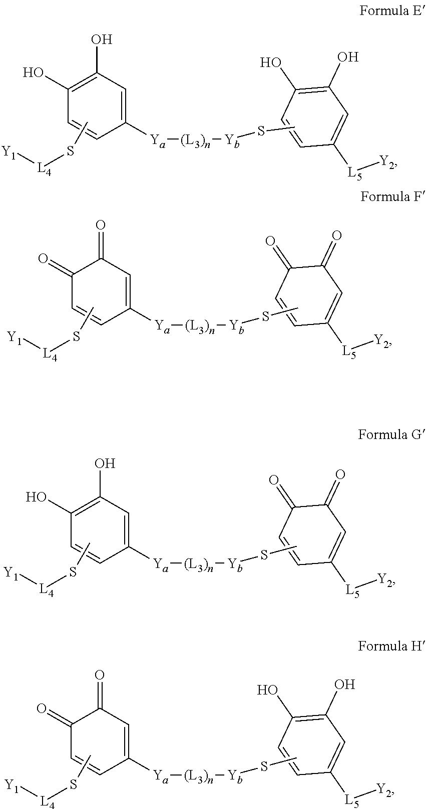

wherein:
Yₐ is a group derived from a first polypeptide comprising a first terminal tag comprising (X₁)ₘ₁X₂X₃X₄X₅Y, wherein X₁-X₅ is any amino acid provided that no more than two amino acids of X₂, X₃, X₄, and X₅ are aspartate or glutamate and wherein m1 is an integer greater than or equal to 0, or X₁X₂X₃, wherein X₁-X₃ is any amino acid provided that at least one amino acid of X₁-X₃ is a tyrosine, wherein the phenol of the tyrosine is oxidized to form:

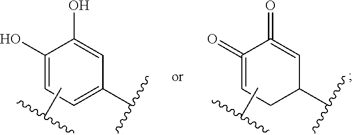

Y_{b} is a second polypeptide;
n is an integer greater than 0;
L₃ is an optional first linker;
L₄ is an optional second linker;
L₅ is an optional third linker in Y₂;
Y₁ is a first payload; and
Y₂ is a group derived from a second payload comprising a phenol, wherein the phenol is oxidized to form:

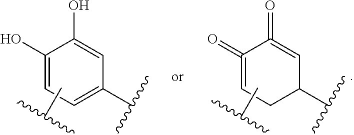

Described herein, in certain embodiments, are conjugates of Formula (J) or Formula (K):

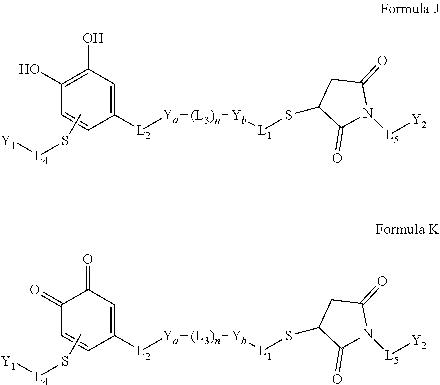

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine;
Y_{b} is a second polypeptide;
n is an integer greater than 0;
L₁ is an optional first linker;
L₂ is an optional second linker;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula J′ or Formula K′:

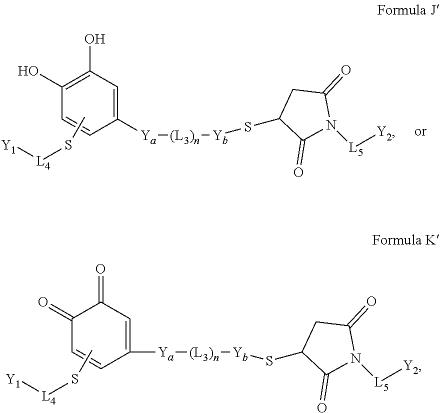

wherein:
Yₐ is a group derived from a first polypeptide comprising a first terminal tag comprising (X₁)ₘ₁X₂X₃X₄X₅, wherein X₁-X₅ is any amino acid provided that at least one amino acid of X₁-X₅ is a tyrosine, or X₁X₂X₃, wherein X₁-X₃ is any amino acid provided that at least one amino acid of X₁-X₃ is a tyrosine, wherein the phenol of the tyrosine is oxidized to form:

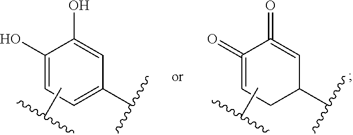

Y_{b} is a second polypeptide;
n is an integer greater than 0;
L₃ is an optional first linker;
L₄ is an optional second linker;
L₅ is an optional third linker;
Y₁ is a first payload; and
Y₂ is a second payload.

Described herein, in certain embodiments, are conjugates of Formula L, Formula M, Formula N, and Formula 0:

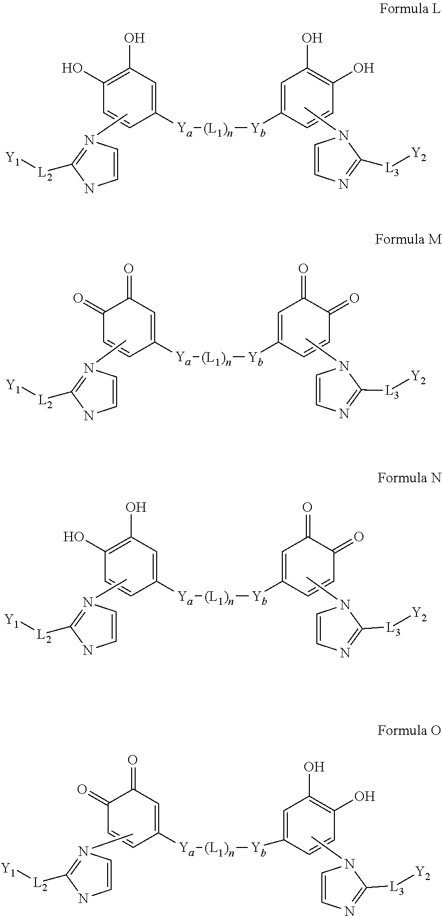

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, wherein Yₐ is linked to “S” through the tyrosine or a portion thereof;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof, wherein Y_{b} is linked to “S” through the tyrosine or a portion thereof;
n is an integer greater than 0;
L₁ is an optional third linker;
L₂ is an optional fourth linker;
L₃ is an optional fifth linker;
Y₁ is a first payload; and
Y₂ is a second payload.

Described herein, in certain embodiments, are conjugates of Formula L, Formula M, Formula N, and Formula O:

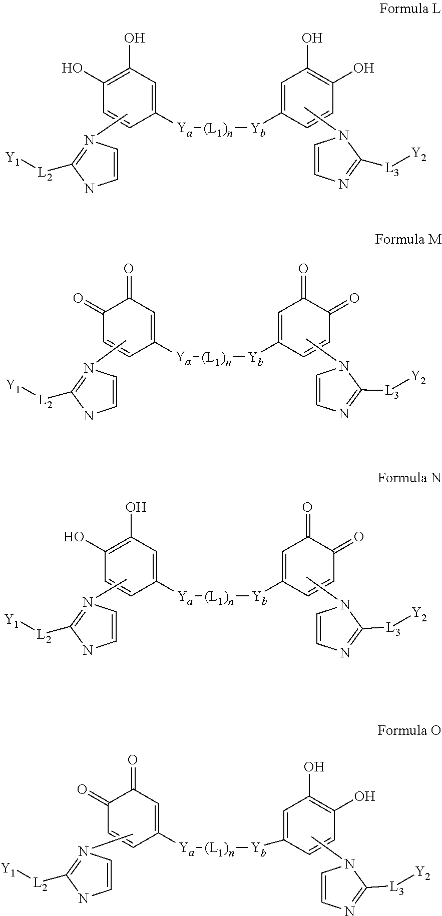

wherein:
Yₐ is group derived from a first polypeptide comprising a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine, wherein the phenol of the tyrosine is oxidized to form:

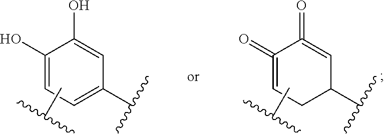

Y_{b} is a group derived from a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0, wherein the phenol of the tyrosine is oxidized to form:

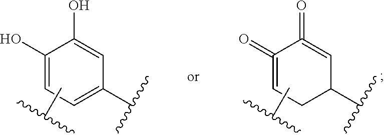

n is an integer greater than 0;
L₁ is an optional first linker;
L₂ is an optional second linker;
L₃ is an optional third linker;
Y₁ is a first payload; and
Y₂ is a second payload.

Described herein, in certain embodiments, are conjugates of Formula L-I, Formula M-I, Formula N-I, and Formula O-I:

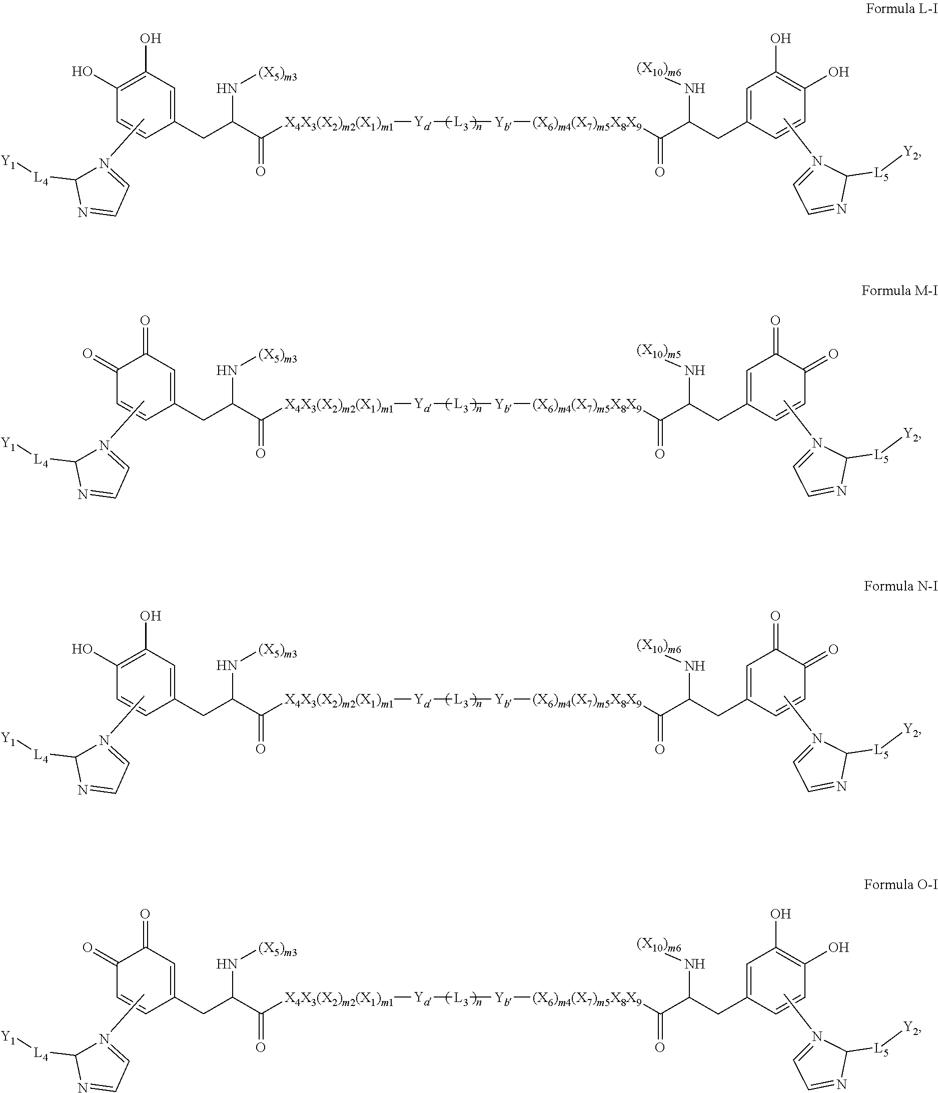

Y_{a′} is a first polypeptide,
each of X₁, X₂, X₃, X₄, and X₅ is independently any amino acid, wherein m1, m2, and m3 are each an integer greater than or equal to 0;
Y_{b′} is a second polypeptide,
each of X₆, X₇, X₈, X₉, and X₁₀ is independently any amino acid, wherein m4, m5, and m6 are each an integer greater than or equal to 0, and wherein the —(X₁)ₘ₁(X₂)ₘ₂X₃X₄-moiety is different from the —(X₆)ₘ₁(X₇)ₘ₂X₈ X₉-moiety;
n is an integer greater than 0;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula P, Formula Q, Formula R, and Formula S:

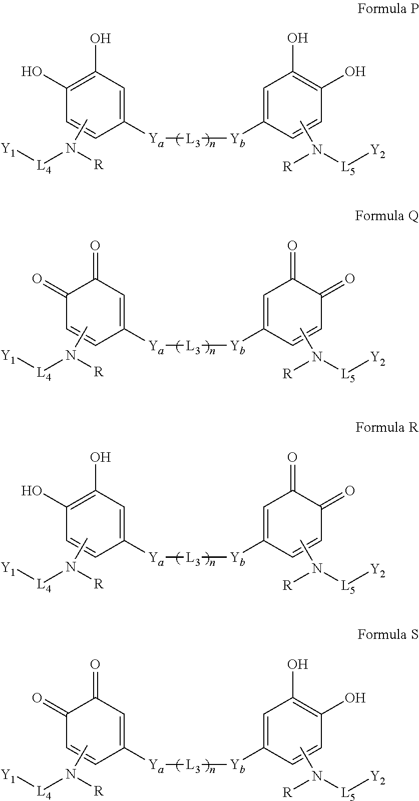

wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁—X₃ is any amino acid provided that at least one amino acid of X₁-X₃ is a tyrosine or a portion thereof, wherein Yₐ is linked to “S” through the tyrosine or a portion thereof;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof, wherein Y_{b} is linked to “S” through the tyrosine or a portion thereof,
n is an integer greater than 0;
L₃ is an optional third linker;
L₄ is an optional fourth linker;
L₅ is an optional fifth linker;
Y₁ is a first payload; and
   Y₂ is a second payload.

Described herein, in certain embodiments, are conjugates of Formula P, Formula Q, Formula R, and Formula S:

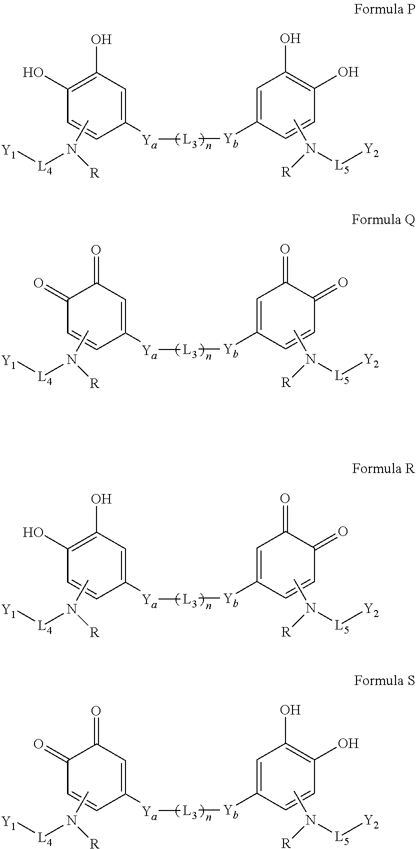

wherein:
Yₐ is a group derived from a first polypeptide comprising a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0; or X₁X₂X₃, wherein X₁-X₃ is any amino acid provided that at least one amino acid of X₁-X₃ is a tyrosine, wherein the phenol of the tyrosine is oxidized to form:

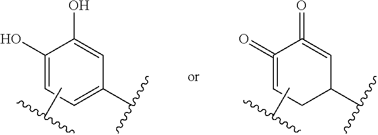

Y_{b} is a group derived from a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0, wherein the phenol of the tyrosine is oxidized to form:

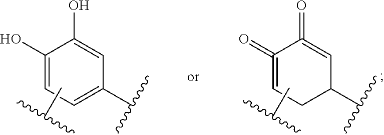

n is an integer greater than 0;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula P-I, Formula Q-I, Formula R-I, and Formula S-I:

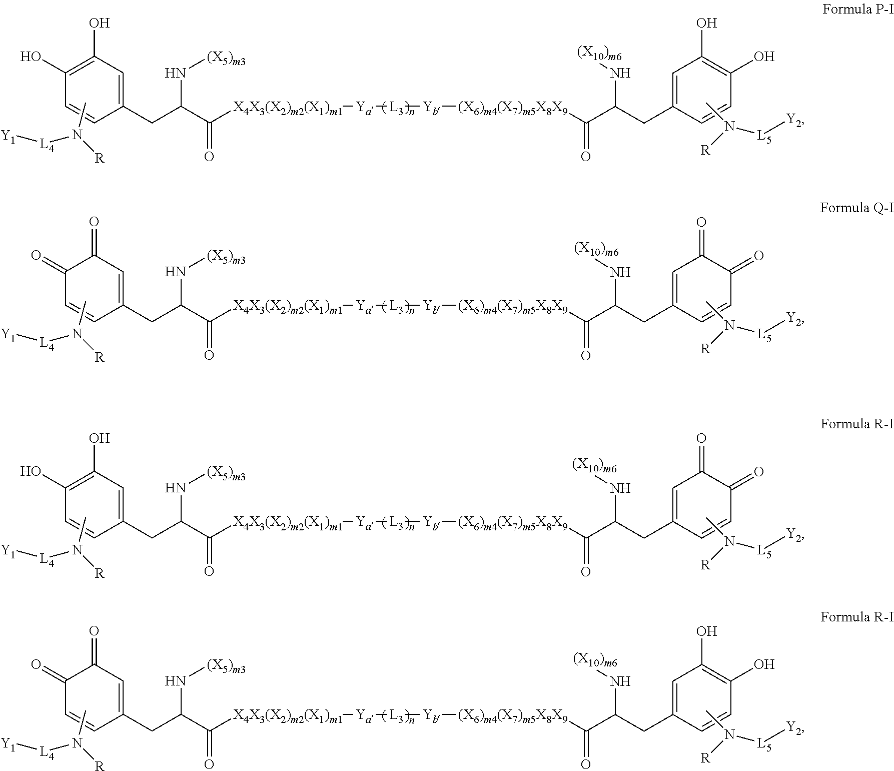

Y_{a′} is a first polypeptide,
each of X₁, X₂, X₃, X₄, and X₅ is independently any amino acid, wherein m1, m2, and m3 are each an integer greater than or equal to 0;
Y_{b′} is a second polypeptide,
each of X₆, X₇, X₈, X₉, and X₁₀ is independently any amino acid, wherein m4, m5, and m6 are each an integer greater than or equal to 0, and wherein the —(X₁)ₘ₁(X₂)ₘ₂X₃X₄-moiety is different from the —(X₆)ₘ₁(X₇)ₘ₂X₈ X₉-moiety;
n is an integer greater than 0;
L₃ is an optional third linker;
L₄ is an optional fourth linker;
L₅ is an optional fifth linker;
Y₁ is a first payload; and
Y₂ is a second payload.

Described herein, in certain embodiments, are conjugates of Formula T, Formula U, Formula V, and Formula W:

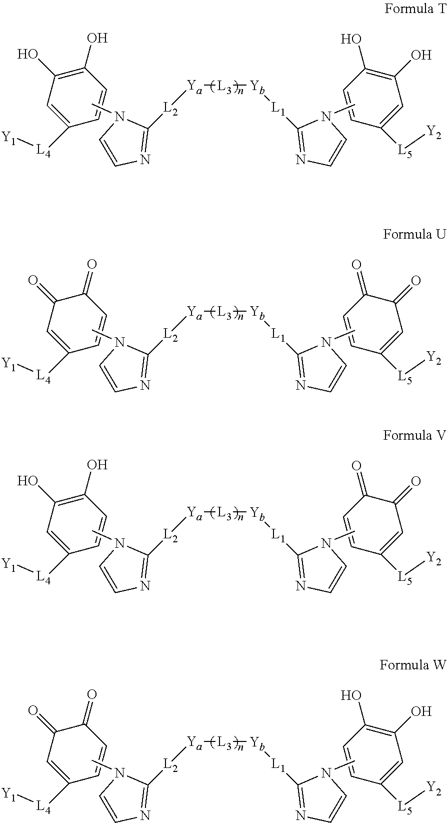

wherein:
Yₐ is a first polypeptide;
Y_{b} is a second polypeptide;
n is an integer greater than 0;
L₃ is an optional third linker;
L₄ is an optional fourth linker;
L₅ is an optional fifth linker in Y₂;
Y₁ is a first payload comprising a phenol, wherein the phenol is linked to imidazole; and
Y₂ is a second payload comprising a phenol, wherein the phenol is linked to imidazole.

Described herein, in certain embodiments, are conjugates of Formula X, Formula Y, Formula Z, and Formula AA:

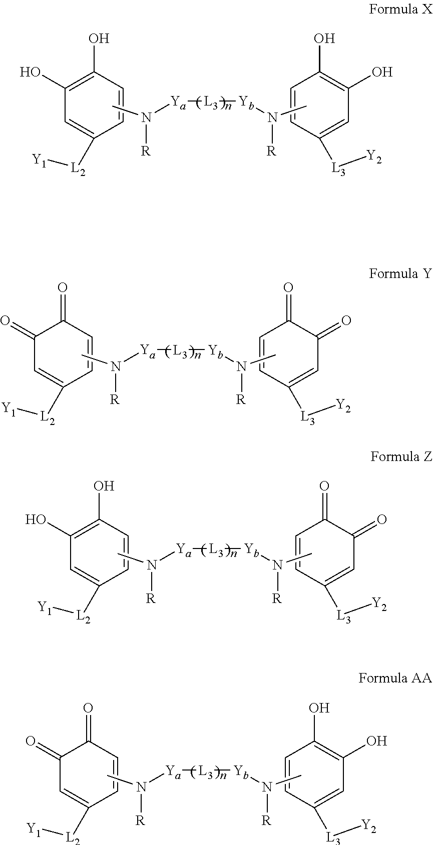

wherein:
Yₐ is a first polypeptide;
Y_{b} is a second polypeptide;
n is an integer greater than 0;
L₁ is an optional first linker;
L₂ is an optional second linker;
L₃ is an optional third linker;
Y₁ is a first payload comprising a phenol, wherein the phenol is linked to “N,” and
Y₂ is a second payload comprising a phenol, wherein the phenol is linked to “N.”

In some embodiments, the tyrosine or a portion thereof is selected from the group

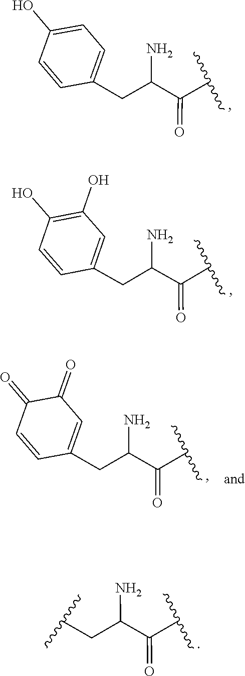 [SMILES: *C(=O)C(N)Cc1ccc(O)cc1 ; *C(=O)C(N)Cc1ccc(O)c(O)c1 ; *C(=O)C(N)CC1=CC(=O)C(=O)C=C1 ; *CC(N)C(*)=O]

In some embodiments, Yₐ comprises a first terminal tag. In some embodiments, the first terminal tag comprises (X₁)ₘ₁X₂X₃X₄X₅, wherein X₁-X₅ is any amino acid provided that at least one amino acid of X₁-X₅ is a tyrosine, and wherein m1 is an integer greater than or equal to 0. In some embodiments, the first terminal tag comprises (X₁)ₘ₁X₂X₃X₄X₅Y, wherein m1 is an integer greater than or equal to 0. In some embodiments, the first terminal tag comprises Y(X₁)ₘ₁X₂X₃X₄X₅, wherein m1 is an integer greater than or equal to 0. In some embodiments, the first terminal tag comprises X₁X₂X₃, wherein X₁-X₃ is any amino acid provided that at least one amino acid of X₁-X₃ is a tyrosine.

In some embodiments, the first terminal tag comprises at least two amino acids of aspartate (D), glutamate (E), arginine (R), and lysine (K). In some embodiments, the first terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, Y_{b} comprises a second terminal tag. In some embodiments, Y_{b} comprises a second terminal tag different from the first terminal tag. In some embodiments, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0. In some embodiments, the second terminal tag comprises (X₆)ₘ₂X₇X₈X₉X₁₀Y, wherein m2 is an integer greater than or equal to 0. In some embodiments, the second terminal tag comprises Y(X₆)ₘ₂X₇X₈X₉X₁₀, wherein m2 is an integer greater than or equal to 0. In some embodiments, the second terminal tag comprises X₁X₂X₃, wherein X₁—X₃ is any amino acid provided that at least one amino acid of X₁-X₃ is a tyrosine.

In some embodiments, the second terminal tag comprises at least two amino acids of aspartate (D), glutamate (E), arginine (R), and lysine (K). In some embodiments, the second terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, the first terminal tag comprises SGGY, SGY, SGGGY, ESY, EESY, ESSY, ESSSY, SESY, SGGGY, EESY, SSSY, SNNY, SSNY, EGGY, SESY, or SEGY.

In some embodiments, the second terminal tag comprises EEEY, EEEEY, SSEEY, SSSEY, SSSSY, or SSNNY.

In some embodiments, the first terminal tag comprises SGGY or SGY and the second terminal tag comprises EEEY.

In some embodiments the first terminal tag comprises SEY and the second terminal tag comprises SSEEY or SSSEY.

In some embodiments, the first terminal tag comprises SGGGY and the second terminal tag comprises EEEEY.

In some embodiments, the first terminal tag comprises ESSY and the second terminal tag comprises SSEEY.

In some embodiments, the first terminal tag comprises ESSY and the second terminal tag comprises SSSEY.

In some embodiments, Yₐ, Y_{b′} or both Yₐ and Y_{b} are glycosylated. In some embodiments, Yₐ, Y_{b′} or both Yₐ and Y_{b} comprise a non-terminal tyrosine.

In some embodiments, Yₐ is a heavy chain variable region (VH) of an antibody or antibody fragment, a light chain variable region (VL) of an antibody or antibody fragment, a heavy chain of an antibody or antibody fragment, a light chain of an antibody or antibody fragment, a constant chain of an antibody or antibody fragment, a peptide, or a cyclic peptide.

In some embodiments, Y_{b} is a heavy chain variable region (VH) of an antibody or antibody fragment, a light chain variable region (VL) of an antibody or antibody fragment, a heavy chain of an antibody or antibody fragment, a light chain of an antibody or antibody fragment, a constant chain of an antibody or antibody fragment, a peptide, or a cyclic peptide.

In some embodiments, Yₐ and Y_{b} are attached via L₃. In some embodiments, L₃ comprises a peptide sequence, a dimerization and docking domain, a leucine zipper, or knobs-into-holes. In some embodiments, L₃ comprises a peptide bond, a disulfide bond, a maleimide bond, thioether bond, an azide-alkyne cycloaddition, a cystinyl-dopa, or a hydrogen bond. In some embodiments, L₃ is a linker. In some embodiments, the linker comprises a sequence selected from the group consisting of (GS)n3, (G2S)n3, (G3S)n3, (G4S)n3, (G)n3, (GGSGGD)n3, (GGSGGE)n3, (GGGSGSGGGGS)n3, and (GGGGGPGGGGP)n3 and wherein n3 is an integer from 2 to 20.

In some embodiments, L₃ comprises a terminal tyrosine.

In some embodiments, n is 1, 2, 3, 4, or 5. In some embodiments, n is 1.

In some embodiments, Y₁ and Y₂ are the same payload. In some embodiments, Y1 and Y₂ are different payloads. In some embodiments, Y₁ and Y₂, or both Y₁ and Y₂ is a small molecule.

In some embodiments, the small molecule is selected from the group consisting of deruxtecan, exatecan, FL118, irinotecan, topotecan, SN-38, rubitecan, belotecan, lurototecan, gimatecan, diflomotecan, karenitecan, silatecan, namitecan, elomotecan, DRF-1042, delimotecan, NSC606985, chimmitecan, ZBH-1205, auristatin (MMAE, MMAF, MMAG, MMAH) calicheamicin, doxorubicin, taxol and taxol derivatives, maytansinoids (DM1-4), pyrolodiazepines (PBDs), tubulysins, eribulin, anthramycin, duocarmycin, anthracycline, and camptothecin (CPT), including the lactone and carboxylate forms of CPT.

In some embodiments, Y₁ and Y₂, or both Y₁ and Y₂ comprises a nucleic acid, an immune agonist, a peptide, a cytokine, or a binding domain.

In another aspect, provided herein is a polypeptide comprising a first terminal tag comprising (X₁)ₘ₁X₂X₃X₄X₅, wherein X₁-X₅ is any amino acid provided that at least one amino acid of X₁-X₅ is a tyrosine, or X₁X₂X₃, wherein X₁-X₃ is any amino acid provided that at least one amino acid of X₁-X₃ is a tyrosine; and wherein m1 is an integer greater than or equal to 0, and a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0. In some embodiments, the polypeptide comprises a non-terminal tyrosine. In some embodiments, the polypeptide is modified to expose the non-terminal tyrosine to be accessible to an enzyme. In some embodiments, the modification includes mutating the sequence of the polypeptide to create an aglycosylated polypeptide. In some embodiments, the polypeptide is deglycosylated by routine methods known in the art.

In another aspect disclosed herein is a composition of first polypeptide and a second polypeptide, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine, or X₁X₂X₃, wherein X₁-X₃ is any amino acid provided that at least one amino acid of X₁-X₃ is a tyrosine; and wherein m1 is an integer greater than or equal to 0, and a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0.

In some embodiments, the first polypeptide and/or the second polypeptide comprise a non-terminal tyrosine.

In some embodiments, the first polypeptide and/or the second polypeptide is modified to expose the non-terminal tyrosine to be accessible to an enzyme.

### Linkers

Linker sequences can be used to separate different components of the conjugates described herein. In some embodiments, the conjugates described herein comprise linkers L₁, L₂, L₃, L₄, and/or L₅.

In some embodiments, any one of the linkers comprises at least 5 to about 50 amino acids. In some embodiments, the linkers comprise about 5 to about 50 amino acids, about 5 to about 45 amino acids, about 5 to about 40 amino acids, about 5 to about 35 amino acids, about 5 to about 30 amino acids, about 5 to about 25 amino acids, about 5 to about 20 amino acids, about 5 to about 15 amino acids, about 5 to about 10 amino acids, about 10 to about 50 amino acids, about 15 to about 50 amino acids, about 20 to about 50 amino acids, about 25 to about 50 amino acids, about 30 to about 50 amino acids, about 35 to about 50 amino acids, about 40 to about 50 amino acids, or about 45 to about 50 amino acids.

In some embodiments, any one of the linkers comprises a sequence selected from the group consisting of (GS)n, (G2S)n, (G3 S)n, (G4S)n, and (G)n, and wherein n is an integer from 2 to 20. In some embodiments, n is an integer from 2 to 18, from 2 to 16, from 2 to 14, from 2 to 12, from 2 to 10, from 2 to 8, from 2 to 6, from 2 to 4, from 4 to 20, from 6 to 20, from 8 to 20, from 10 to 20, from 12 to 20, from 14 to 20, from 16 to 20, or from 18 to 20.

In some embodiments, any one of the linkers comprises a sequence selected from the group consisting of (GGSGGD)n or (GGSGGE)n, and wherein n is an integer from 2 to 6.

In some embodiments, any one of the linkers comprises a sequence selected from the group consisting of (GGGSGSGGGGS)n and (GGGGGPGGGGP)n, and wherein n is an integer from 1 to 3.

In some embodiments, any one of the linkers comprises a sequence selected from the group consisting of (GX)n, (GGX)n, (GGGX)n, (GGGGX)n, and (GzX)n, wherein z is between 1 and 20, and wherein n is at least 8. In some embodiments, z is between 2 and 18, 2 and 16, 2 and 14, 2 and 12, 2 and 10, 2 and 8, 2 and 6, 2 and 4, 4 and 20, 6 and 20, 8 and 20, 10 and 20, 12 and 20, 14 and 20, 16 and 20, or 18 and 20. In some embodiments, X is serine, aspartic acid, glutamic acid, threonine, or proline.

In some embodiments, a linker in the conjugates disclosed herein in a cleavable linker In some embodiments, at least one of L₁ L₂, L₃, L₄, or L₅ is a cleavable linker. In some embodiments, the cleavable linker is an electrophilically cleavable linker, a nucleophilically cleavable linker, a photocleavable linker, a metal cleavable linker, an electrolytically-cleavable linker, an acid cleavable linker, or a proteolytically cleavable linker. In some embodiments, the cleavable linker is cleavable under reductive and/or oxidative conditions. In some embodiments, the cleavable linker is cleavable under acidic conditions. In some embodiments, the cleavable linker is cleaved by an enzyme. In certain cases, the cleavable linker is a linker that is cleaved under reducing conditions. In some embodiments, the cleavable linker is cleaved rapidly by glutathione reduction. In some embodiments, the cleavable linker comprises a disulfide bond. In some embodiments, the cleavable linker is cleaved by a physical stimulus. In some embodiments, the cleavable linker is photocleavable.

In some embodiments, Li, L₂, L₃, L₄, or L₅ is an acid-labile linker. In some embodiments, the linker cleaves at a pH of 6 or less, such as, 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, 4.9, 4.85, 4.80, 4.75, 4.7, 4.65, 4.6, 4.55, 4.5 or even less.

In some embodiments, L₁, L₂, L₃, L₄, or L₅ is a photocleavable linker. Suitable photocleavable linkers include ortho-nitrobenzyl-based linkers, phenacyl linkers, alkoxybenzoin linkers, chromium arene complex linkers, NpSSMpact linkers and pivaloylglycol linkers, as described in Guillier et al. (Chem. Rev. 2000 1000:2091-2157).

In some embodiments, L1, L2, L3, L4 or L5 is a proteolytically cleavable linker. The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

In some embodiments, the proteolytically cleavable linker comprises a matrix metalloproteinase cleavage site, e.g., a cleavage site for a MMP selected from collagenase-1,-2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP).

In some embodiments, the cleavable linker comprises a disulfide bond and is cleavable under reducing conditions, e.g., using b-mercaptoethanol, cysteine-HCl, Iris (2-carboxyethyl) phosphine hydrochloride, or another reducing agent.

In some embodiments, the cleavable linker comprises a dipeptide. In some embodiments, the dipeptide is a valine-citrulline (Val-Cit) dipeptide or a valine-lysine dipeptide.

In some embodiments, the cleavable linker comprises a tetrapeptide. In some embodiments, the tetrapeptide is a glycine-glycine-phenylalanine-glycine tetrapeptide (GGFG).

In some embodiments, the cleavable linker is PABC (p-aminobenzyl alcohol), glucuronide, or MABC (m-aminobenzyl alcohol).

In some embodiments, the cleavable linker is a valine-citrulline (Val-Cit) PABC (p-aminobenzyl alcohol) (VC-PABC) linker. In some embodiments, the VC-PABC linker has the structure:

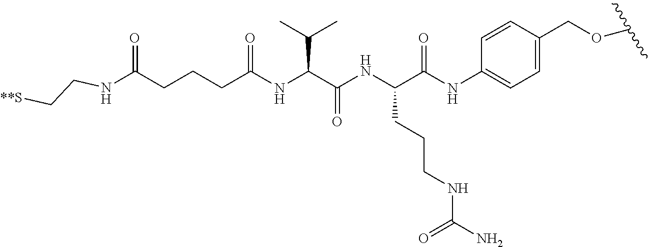

wherein the point of attachment of the payload is “ [symbol] .”

In some embodiments, the disclosure provides a compound of formula L-I,

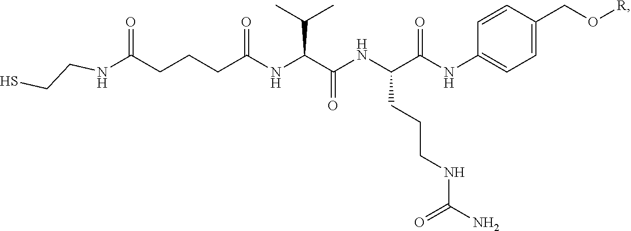

where R is a payload.

In some embodiments, the compound is selected from the compound is selected from the group consisting of:

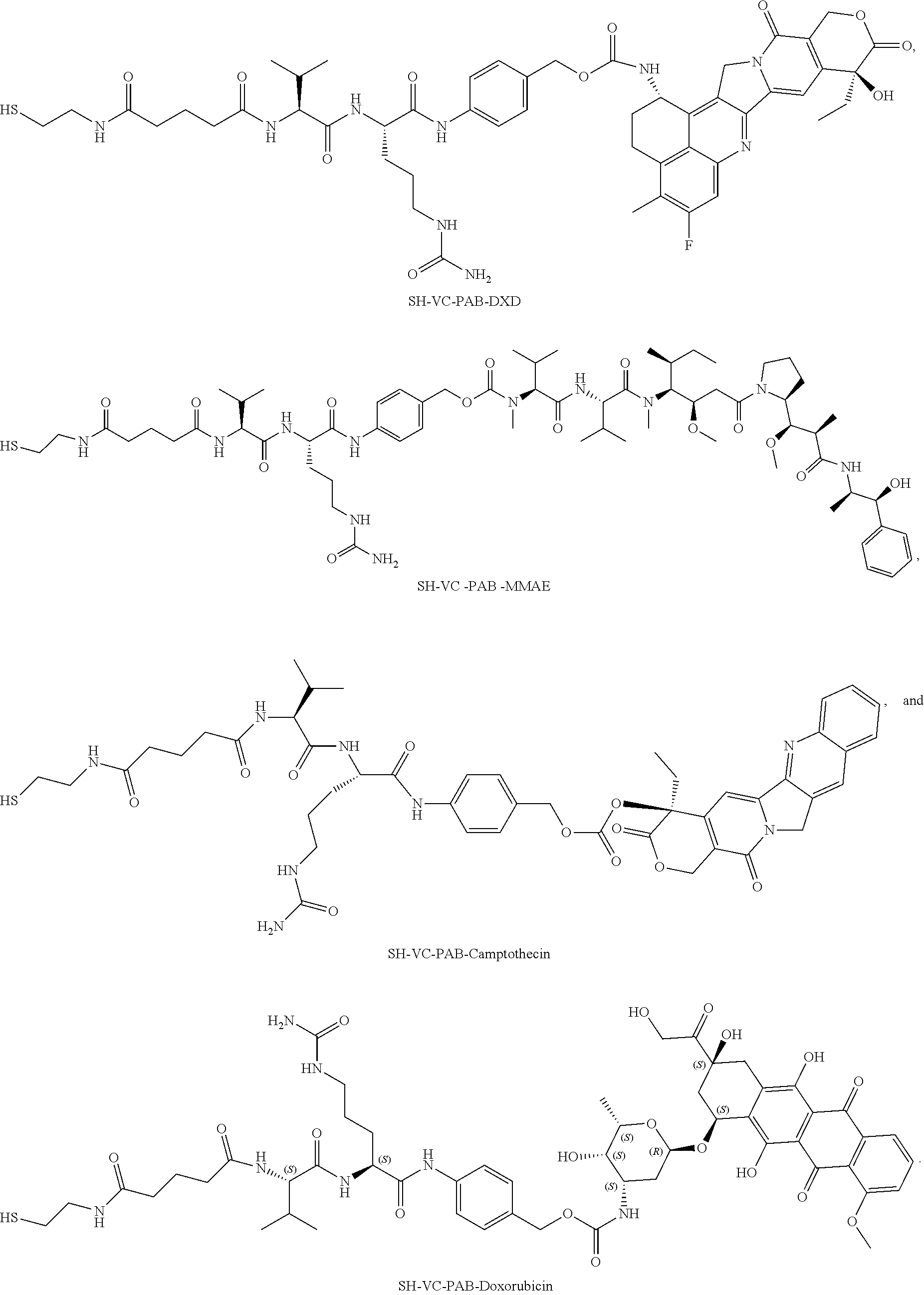

In some embodiments, the cleavable linker further include a polyethylene glycol group (i.e., a pegylated group), a sugar group, or a modification that increases hydrophilicity. In some embodiments, a cleavable linker including a sugar group is a glucuronide linker. In some embodiments, a modification that increases hydrophilicity include a longer peptide chain, inclusion of charged residues, additional PEG spacers, branched PEG substituents, peptoid bonds, solfonate bonds, glucuronide linkers, and methylation of tertiary amines to produce cations.

### Polypeptides and Biomolecules

Described herein, in certain embodiments, are conjugates comprising one or more polypeptides or biomolecules.

In some embodiments, the polypeptide is an enzyme, an antibody, a structural polypeptide, a ligand for a receptor, or a receptor.

In some embodiments, the polypeptide is an antibody. In some embodiments, the antibody is a single chain Fv (scFv). In some embodiments, the polypeptide is a heavy chain variable region (VH), a light chain variable region (VL), a heavy chain, a light chain, a constant chain of an antibody or antibody fragment. Other antibody-based recognition domains (cAb VFiFi (camelid antibody variable domains) and humanized versions, IgNAR VFi (shark antibody variable domains) and humanized versions, sdAb VFi (single domain antibody variable domains) and “camelized” antibody variable domains are suitable for use. In some embodiments, T-cell receptor (TCR) based recognition domains such as single chain TCR (scTv, single chain two-domain TCR containing nanobody) are also suitable for use.

Biomolecules that are suitable for use in a method or conjugate of the present disclosure include polypeptides, peptides, cyclic peptides, polynucleotides, nucleic acids, aptamers, glycoproteins, small molecules, carbohydrates, lipids, glycolipids, lipoproteins, fatty acids, lipopolysaccharides, sugars, amino acids, organic dyes, synthetic polymers, steroids, nanoparticles, polymers, purines, pyrimidines, derivatives, structural analogs thereof and combinations thereof.

In some embodiments, a biomolecule suitable for conjugating to a payload is a peptide. In some embodiments, the peptide is a peptide ligand or a binding peptide. In some embodiments, the binding peptide can be of different origins, e.g., synthetic, human, mouse, or rat. In some embodiments, a binding peptide may be or have been engineered to include one or more (e.g., two, three, four, or five) solvent-exposed cysteine or lysine residues, which may provide a site for conjugation. In some embodiments, the binding peptides may include only naturally occurring amino acid residues, or may include one or more non-naturally occurring amino acid residues. In some embodiments, binding peptides may be linear or cyclic. In some embodiments, the peptide ligand or binding peptide is a bicyclic peptide. In some embodiments, a binding peptide can be a monospecific peptide or multi-specific peptide (e.g., a bispecific peptide or a trispecific peptide). Bispecific peptide ligands or dual specific peptide ligands may bind one target at a time or bind two targets simultaneously. In some embodiments, a binding peptide is a cysteine motif binding peptide.

As used herein, “binding peptide” refers to peptides having a potential capability of binding other compounds and/or structures (e.g., polypeptides and proteins).

Suitable lipids may include, e.g., 3-N-[(methoxypoly (ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC), cholesterol, dipalmitoylphosphatidylcholine, 3-N-[(w-methoxy poly(ethylene glycol)2000)carbamoyl]-1,2-dimyrestyloxypropylamine, 1,2-dilinoleyloxy-3-N,Ndimethylaminopropane, 1,2-distearoyl-sn-glycero-3-phosphocholine, PEG-cDMA, 1,2-dilinoleyloxy-3-(N;N-dimethyl)aminopropane (DLinDMA), 2,2-dilinoleyl-4-dimethylaminoethyl-[1,3]-dioxolane (DLin-KC2-DMA), and the like.

Suitable biomolecules may include affinity moieties. Suitable affinity moieties may include His5 (TIHIIH) (SEQ ID NO: 34); HisX6 (HIHHH) (SEQ ID NO: 35); c-myc (EQKLISEEDL) (SEQ ID NO:36); Flag (DYKDDDDK) (SEQ ID NO:37); StrepTag (WSHPQFEK) (SEQ ID NO:38); hemagglutinin, e.g., HA Tag (YPYDVPDYA) (SEQ ID NO:39); glutathione-S-transferase (GST); thioredoxin; cellulose binding domain, RYIRS (SEQ ID NO:40); Phe-His-His-Thr (SEQ ID NO:41); chitin binding domain; S-peptide; T7 peptide; SH2 domain; C-end RNA tag, WEAAAREACCRECCARA (SEQ ID NO:42); metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit. SI 00 proteins parvalbumin, calbindin D9K, calbindin D28K, and calretinin; biotin; streptavidin; MyoD; leucine zipper polypeptides; and maltose binding protein. In some embodiments, a suitable biomolecule is biotin.

In some embodiments, a biomolecule suitable for conjugating to a payload is a dimerization domain. Non-limiting examples of suitable dimerization domains include polypeptides of the following dimerization pairs:
a) FK506 binding protein (FKBP) and FKBP;
b) FKBP and calcineurin catalytic subunit A (CnA);
c) FKBP and cyclophilin;
d) FKBP and FKBP-rapamycin associated protein (FRB);
e) gyrase B (GyrB) and GyrB;
f) dihydrofolate reductase (DHFR) and DHFR;
g) DmrB and DmrB;
h) PYL and ABI;
i) Cry2 and CIB 1; and
j) GAI and GID1.

In some cases, a biomolecule suitable for conjugating to a payload is a member of a specific binding pair. Specific binding pairs include, e.g.: i) antibody-antigen; ii) cell adhesion molecule-extracellular matrix; iii) ligand-receptor; iv) biotin-avidin; and the like.

Suitable synthetic polymers include, but are not limited to, polyalkylenes such as polyethylene and polypropylene and polyethyleneglycol (PEG); poly chloroprene; polyvinyl ethers such as poly(vinyl acetate); polyvinyl halides such as poly(vinyl chloride); polysiloxanes; polystyrenes; polyurethanes; polyacrylates such as poly(methyl (meth) acrylate), poly(ethyl (meth)acrylate), poly(n-butyl (meth)acrylate), poly(isobutyl (meth)acrylate), poly(tert-butyl (meth)acrylate), poly(hexyl (meth)acrylate), poly(isodecyl (meth) acrylate), poly(lauryl (meth)acrylate), poly(phenyl (meth)acrylate), poly(methyl acrylate), poly(isopropyl acrylate), poly (isobutyl acrylate), and poly(octadecyl acrylate); polyacrylamides such as poly (acrylamide), poly (methacrylamide), poly(ethyl acrylamide), poly (ethyl methacrylamide), poly(N-isopropyl acrylamide), poly(n, iso, and tert-butyl acrylamide); and copolymers and mixtures thereof.

In some embodiments, the biomolecule is a fluorescent protein. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

In some embodiments, the biomolecule is a nucleic acid. In some embodiments, the nucleic acid is a DNA molecule. In some embodiments, the nucleic acid is an RNA molecule. In some embodiments, the nucleic acid comprises both deoxyribonucleotides and ribonucleotides. In some embodiments, the nucleic acid is a single-stranded DNA molecule. In some embodiments, the nucleic acid is a double-stranded DNA molecule. In some embodiments, the nucleic acid is a single-stranded RNA molecule. Suitable nucleic acids include, e.g., a small interfering RNA (siRNA), a short hairpin RNA (shRNA), a microRNA (miRNA), a ribozyme, a aptamer, and the like. Suitable nucleic acids include nucleic acids that are or act as siRNAs or other RNA interference reagents (RNAi agents or iRNA agents), shRNA, antisense oligonucleotides, self-cleaving RNAs, ribozymes, fragment thereof and/or variants thereof (such as Peptidyl transferase 23S rRNA, RNase P, Group I and Group II introns, GIR1 branching ribozymes, Leadzyme, Hairpin ribozymes, Hammerhead ribozymes, HDV ribozymes, Mammalian CPEB3 ribozyme, VS ribozymes, glmS ribozymes, CoTC ribozyme, etc.), microRNAs, microRNA mimics, supermirs, aptamers, antimirs, antagomirs, U1 adaptors, triplex-forming oligonucleotides, RNA activators, long non-coding RNAs, short non-coding RNAs (e.g., piRNAs), immunomodulatory oligonucleotides (such as immunostimulatory oligonucleotides, immunoinhibitory oligonucleotides), GNA, LNA, ENA, PNA, TNA, HNA, TNA, XNA, HeNA, CeNA, morpholinos, G-quadruplex (RNA and DNA), antiviral oligonucleotides, and decoy oligonucleotides. Nucleic acids can be of any length, and can include one or more of a modified ribonucleotide base, a modified deoxyribonucleotide base, a modified deoxyribose, a modified ribose, and a modified backbone linkage (e.g., a phosphorothioate linkage).

In some embodiments, the biomolecule is an oligonucleotide. In some embodiments, an oligonucleotide can include an oligonucleotide complementary to a gene sense sequence, a pre-mRNA sense sequence, and/or mRNA sense sequence, or a portion thereof. In some embodiments, an oligonucleotide can include an oligonucleotide of a gene sense sequence, a pre-mRNA sense sequence, and/or mRNA sense sequence, or a portion thereof. In some embodiments, oligonucleotides described herein can also be nucleotide chemical analog-based compounds capable of binding to a gene sense sequence, a pre-mRNA sense sequence, and/or an mRNA sense sequence, or a portion thereof. In some embodiments, the oligonucleotide is a sense oligonucleotide. In some embodiments, the oligonucleotide is an antisense oligonucleotide. In some embodiments, the antisense oligonucleotide can be a single-stranded nucleic acid molecule.

In some embodiments, the oligonucleotide may be an oligonucleotide sequence of 5 to 100 nucleotides in length, for example, 10 to 40 nucleotides in length, for example, 14 to 40 nucleotides in length, for example, 10 to 30 nucleotides in length, for example, 14 to 30 nucleotides in length, for example, 14 to 25 nucleotides in length, for example, 15 to 22 oligonucleotides in length, for example, 16 to 40 nucleotides in length, for example, 18 to 24 nucleotides in length, for example 20 to 40 nucleotides in length, or for example, 20 to 24 nucleotides in length. An oligonucleotide may comprise an oligonucleotide sequence complementary to one or more than one portion of an mRNA sequence.

In some embodiments, an oligonucleotide comprises one or more ribonucleotides, one or more deoxyribonucleotides, or a mixture of ribonucleotides and deoxyribonucleotides.

In some embodiments, an oligonucleotide comprises one or more modified nucleosides, for example, 5-methylcytidine, 5-methyl-2′-deoxycytidine, deoxycytidine, 5-methyl-2′-deoxycytidine 5′-monophosphate, or 5-methyl-2′-deoxycytidine-5′-monophosphorothioate. In certain embodiments, an oligonucleotide comprises one or more modified nucleosides, for example, 2′-O-methylcytidine, 2′-O-methylguanosine, 2′-O-methylthymidine, 2′-O-methyluridine, or 2′-O-methyladenosine. In some embodiments, an oligonucleotide comprises one or more modified nucleotide, for example, 5-methyl cytosine or 5-methylguanine. In some embodiments, an oligonucleotides comprises one or more modified nucleotides, for example, 2′-O-(2-methoxyethyl) nucleosides, 2′-deoxy-2′-fluoro nucleosides, or 2′-fluoro-o-D-arabinonucleosides.

In some embodiments, an oligonucleotide comprises bridged nucleic acids, locked nucleic acids (LNA), constrained ethyl (cET) nucleic acids, tricyclo-DNAs (tcDNA), 2′-0,4′-C-ethylene linked nucleic acids (ENA), or peptide nucleic acids (PNA).

In some embodiments, an oligonucleotide may have a modified linkage, such as a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, and an aminoalkylphosphoramidate linkage, a thiophosphoramidate linkage, a thionoalkylphosphonate linkage, a thionoalkylphosphotriester linkage, a thiophosphate linkage, a selenophosphate linkage, and/or a boranophosphate linkage.

### Payloads of the Disclosure

Described herein, in certain embodiments, are conjugates comprising one or more payloads.

In some embodiments, the conjugates comprise multiple payloads. In some embodiments, the conjugates comprise 1, 2, 3, 4, 5, 6, 7, 8, 9, 10, or more than 10 payloads. In some embodiments, the conjugates comprise 1 payload. In some embodiments, the conjugates comprise 2 payloads. In some embodiments, the conjugates comprise 3 payloads. In some embodiments, the conjugates comprise 4 payloads. In some embodiments, the conjugates comprise 5 payloads. In some embodiments, the conjugates comprise 6 payloads. In some embodiments, the conjugates comprise 7 payloads. In some embodiments, the conjugates comprise 8 payloads. In some embodiments, the conjugates comprise 9 payloads. In some embodiments, the conjugates comprise 10 payloads. In some embodiments, the conjugates comprise multiple payloads conjugated to different chains of an antibody (e.g., a light chain and a heavy chain). In some embodiments, the conjugates comprise multiple payloads that are the same. In some embodiments, the conjugates comprise multiple payloads that are different. In some embodiments, the conjugates comprise multiple payloads that are different, wherein more than one of each of the different payloads are the same. For example, the conjugates comprise two tubulin inhibitor payloads and two topoisomerase inhibitor payloads.

The payloads can be naturally occurring, or may be synthetically or recombinantly produced, and may be isolated, substantially purified, or present within the native milieu of the unmodified molecule upon which the moiety-containing payloads is based (e.g., on a cell surface or within a cell, including within a host animal, e.g., a mammalian animal, such as a murine host (e.g., rat, mouse), hamster, canine, feline, bovine, swine, and the like). In some embodiments, the payloads are present in vitro in a cell-free reaction. In other embodiments, the payloads are present in a cell and/or displayed on the surface of a cell. In many embodiments of interest, the payloads are in a living cell; on the surface of a living cell; in a living organism, e.g., in a living multicellular organism. Suitable living cells include cells that are part of a living multicellular organism; cells isolated from a multicellular organism; immortalized cell lines; and the like.

The payloads may be composed of D-amino acids, L-amino acids, or both, and may be further modified, either naturally, synthetically, or recombinantly, to include other moieties. For example, the payloads may be lipoproteins, glycoproteins, or other such modified proteins.

In some embodiments, the payloads comprise at least one thiol moiety for reaction with at least one polypeptide comprising a reactive moiety, but may comprise 2 or more, 3 or more, 5 or more, 10 or more thiol moieties. The number of thiol moieties that may be present in a target molecule will vary according to the intended application of the modified target molecule of the reaction, the nature of the target molecule itself, and other considerations which will be readily apparent to the ordinarily skilled artisan in practicing the methods as disclosed herein.

The payloads can be modified to comprise a thiol moiety at the point at which linkage to the polypeptides comprising a reactive moiety is desired. For example, when the payload is a peptide or a polypeptide, the payload may be modified to contain an N-terminal thiol moiety, thereby producing a subject target peptide or polypeptide comprising a thiol moiety. It will be understood that any convenient location on a peptide or a polypeptide substrate may be modified to contain a thiol moiety and thereby produce a target peptide or polypeptide for use in the subject methods.

In some embodiments, the payloads are small molecules.

The payloads comprising a reactive moiety will in some embodiments comprise a small molecule drug, toxin, or other molecule for delivery to a cell. The small molecule drug, toxin, or other molecule will in some embodiments provide for a pharmacological activity. The small molecule drug, toxin, or other molecule will in some embodiments serve as a target for delivery of other molecules.

Small molecule drugs may be small organic or inorganic compounds having a molecular weight of more than 50 Daltons and less than about 2,500 Daltons. Small molecule drugs may comprise functional groups necessary for structural interaction with proteins, particularly hydrogen bonding, and may include at least an amine, carbonyl, hydroxyl or carboxyl group, and may contain at least two of the functional chemical groups. The drugs may comprise cyclical carbon or heterocyclic structures and/or aromatic or polyaromatic structures substituted with one or more of the above functional groups. Small molecule drugs are also found among biomolecules including peptides, saccharides, fatty acids, steroids, purines, pyrimidines, derivatives, structural analogs or combinations thereof.

In some embodiments, the payloads are cancer chemotherapeutic agents, which may include small molecules as described herein.

Suitable cancer chemotherapeutic agents include, e.g., alkylating agents, such as nitrogen mustards (for example, chlorambucil, chlormethine, cyclophosphamide, ifosfamide, and melphalan); nitrosoureas (for example, carmustine, fotemustine, lomustine, and streptozocin); platinum compounds (for example, carboplatin, cisplatin, oxaliplatin, and BBR3464); busulfan; dacarbazine; mechlorethamine; procarbazine; temozolomide; thiotepa; uramustine; antimetabolites, such as folic acid (for example, methotrexate, pemetrexed, and raltitrexed); purine (for example, cladribine, clofarabine, fludarabine, mercaptopurine, and tioguanine); pyrimidine (for example, capecitabine); cytarabine; fluorouracil; gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, rifampicin, hydroxyurea, and mitomycin; topoisomerase inhibitors, such as topotecan and irinotecan; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, axitinib, bexarotene, bevacizumab, bortezomib, celecoxib, denileukin diftitox, erlotinib, estramustine, gefitinib, hydroxycarbamide, imatinib, lapatinib, pazopanib, pentostatin, masoprocol, mitotane, pegaspargase, tamoxifen, sorafenib, sunitinib, emurafenib, vandetanib, and tretinoin. In some embodiments, the cancer chemotherapeutic agent is a topoisomerase inhibitor. In some embodiments, the cancer chemotherapeutic agent is a tubulin inhibitor. In some embodiments, the conjugates comprise two cancer chemotherapeutic agents. In some embodiments, the two cancer chemotherapeutic agents are a topoisomerase inhibitor and a tubulin inhibitor.

In some embodiments, a payload comprising a reactive moiety is selected from the group consisting of deruxtecan, exatecan, FL118, irinotecan, topotecan, SN-38, rubitecan, belotecan, lurototecan, gimatecan, diflomotecan, karenitecan, silatecan, namitecan, elomotecan, DRF-1042, delimotecan, NSC606985, chimmitecan, ZBH-1205, auristatin (MMAE, MMAF, MMAG, MMAH), calicheamicin, doxorubicin, taxol and taxol derivatives, maytansinoids (DM1-4), pyrolodiazepines (PBDs), tubulysins, eribulin, anthramycin, duocarmycin, anthracycline, and camptothecin (CPT), including the lactone and carboxylate forms of CPT. In some embodiments, a payload comprising a reactive moiety is selected from the group consisting of exatecan, auristatin (MMAE, MMAF, MMAG, MMAH), doxorubicin, and camptothecin (CPT). In some embodiments, a payload comprising a reactive moiety comprises an immune stimulating compound. In some embodiments, a payload comprising a reactive moiety comprises a DNA repair inhibitor.

In some embodiments, a payload comprising a reactive moiety is selected from the group consisting of STING agonists including cyclic di-GMP, diABZI and derivatives thereof, TLR7 agonists, TLR8 agonists, and other agonists of immune receptors.

In some embodiments, a payload comprising a reactive moiety comprises one of a pair of binding partners (e.g., a ligand; a ligand-binding portion of a receptor; an antibody; an antigen-binding fragment of an antibody; an antigen; a hapten; a lectin; a lectin binding carbohydrate). For example, the payload can comprise a polypeptide that serves as a viral receptor and, upon binding with a viral envelope protein or viral capsid protein, facilitates attachment of virus to the cell surface on which the biomolecule is displayed.

In some embodiments, the payload is a nucleic acid. In some embodiments, the nucleic acid is a DNA molecule. In some embodiments, the nucleic acid is an RNA molecule. In some embodiments, the nucleic acid comprises both deoxyribonucleotides and ribonucleotides. In some embodiments, the nucleic acid is a single-stranded DNA molecule. In some embodiments, the nucleic acid is a double-stranded DNA molecule. In some embodiments, the nucleic acid is a single-stranded RNA molecule. Suitable nucleic acids include, e.g., a small interfering RNA (siRNA), a short hairpin RNA (shRNA), a microRNA (miRNA), a ribozyme, a aptamer, and the like. Suitable nucleic acids include nucleic acids that are or act as siRNAs or other RNA interference reagents (RNAi agents or iRNA agents), shRNA, antisense oligonucleotides, self-cleaving RNAs, ribozymes, fragment thereof and/or variants thereof (such as Peptidyl transferase 23S rRNA, RNase P, Group I and Group II introns, GIR1 branching ribozymes, Leadzyme, Hairpin ribozymes, Hammerhead ribozymes, HDV ribozymes, Mammalian CPEB3 ribozyme, VS ribozymes, glmS ribozymes, CoTC ribozyme, etc.), microRNAs, microRNA mimics, supermirs, aptamers, antimirs, antagomirs, U1 adaptors, triplex-forming oligonucleotides, RNA activators, long non-coding RNAs, short non-coding RNAs (e.g., piRNAs), immunomodulatory oligonucleotides (such as immunostimulatory oligonucleotides, immunoinhibitory oligonucleotides), GNA, LNA, ENA, PNA, TNA, HNA, TNA, XNA, HeNA, CeNA, morpholinos, G-quadruplex (RNA and DNA), antiviral oligonucleotides, and decoy oligonucleotides. Nucleic acids can be of any length, and can include one or more of a modified ribonucleotide base, a modified deoxyribonucleotide base, a modified deoxyribose, a modified ribose, and a modified backbone linkage (e.g., a phosphorothioate linkage).

In some embodiments, the payload is an oligonucleotide. In some embodiments, an oligonucleotide can include an oligonucleotide complementary to a gene sense sequence, a pre-mRNA sense sequence, and/or mRNA sense sequence, or a portion thereof. In some embodiments, an oligonucleotide can include an oligonucleotide of a gene sense sequence, a pre-mRNA sense sequence, and/or mRNA sense sequence, or a portion thereof. In some embodiments, oligonucleotides described herein can also be nucleotide chemical analog-based compounds capable of binding to a gene sense sequence, a pre-mRNA sense sequence, and/or an mRNA sense sequence, or a portion thereof. In some embodiments, the oligonucleotide is a sense oligonucleotide. In some embodiments, the oligonucleotide is an antisense oligonucleotide. In some embodiments, the antisense oligonucleotide can be a single-stranded nucleic acid molecule.

In some embodiments, the oligonucleotide may be an oligonucleotide sequence of 5 to 100 nucleotides in length, for example, 10 to 40 nucleotides in length, for example, 14 to 40 nucleotides in length, for example, 10 to 30 nucleotides in length, for example, 14 to 30 nucleotides in length, for example, 14 to 25 nucleotides in length, for example, 15 to 22 oligonucleotides in length, for example, 18 to 40 nucleotides in length, for example, 18 to 24 nucleotides in length, for example 20 to 40 nucleotides in length, or for example, 20 to 24 nucleotides in length. An oligonucleotide may comprise an oligonucleotide sequence complementary to one or more than one portion of an mRNA sequence.

In some embodiments, an oligonucleotide comprises one or more ribonucleotides, one or more deoxyribonucleotides, or a mixture of ribonucleotides and deoxyribonucleotides.

In some embodiments, an oligonucleotide comprises one or more modified nucleosides, for example, 5-methylcytidine, 5-methyl-2′-deoxycytidine, deoxycytidine, 5-methyl-2′-deoxycytidine 5′-monophosphate, or 5-methyl-2′-deoxycytidine-5′-monophosphorothioate. In certain embodiments, an oligonucleotide comprises one or more modified nucleosides, for example, 2′-O-methylcytidine, 2′-O-methylguanosine, 2′-O-methylthymidine, 2′-O-methyluridine, or 2′-O-methyladenosine. In some embodiments, an oligonucleotide comprises one or more modified nucleotide, for example, 5-methyl cytosine or 5-methylguanine. In some embodiments, an oligonucleotides comprises one or more modified nucleotides, for example, 2′-O-(2-methoxyethyl) nucleosides, 2′-deoxy-2′-fluoro nucleosides, or 2′-fluoro-o-D-arabinonucleosides.

In some embodiments, an oligonucleotide comprises bridged nucleic acids, locked nucleic acids (LNA), constrained ethyl (cET) nucleic acids, tricyclo-DNAs (tcDNA), 2′-0,4′-C-ethylene linked nucleic acids (ENA), or peptide nucleic acids (PNA).

In some embodiments, an oligonucleotide may have a modified linkage, such as a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, and an aminoalkylphosphoramidate linkage, a thiophosphoramidate linkage, a thionoalkylphosphonate linkage, a thionoalkylphosphotriester linkage, a thiophosphate linkage, a selenophosphate linkage, and/or a boranophosphate linkage.

In some embodiments, a payload is a peptide. In some embodiments, the peptide is a peptide ligand or a binding peptide. In some embodiments, the binding peptide can be of different origins, e.g., synthetic, human, mouse, or rat. In some embodiments, a binding peptide may be or have been engineered to include one or more (e.g., two, three, four, or five) solvent-exposed cysteine or lysine residues, which may provide a site for conjugation. In some embodiments, the binding peptides may include only naturally occurring amino acid residues, or may include one or more non-naturally occurring amino acid residues. In some embodiments, binding peptides may be linear or cyclic. In some embodiments, the peptide ligand or binding peptide is a bicyclic peptide. In some embodiments, a binding peptide can be a monospecific peptide or multi-specific peptide (e.g, a bispecific peptide or a trispecific peptide). Bispecific peptide ligands or dual specific peptide ligands may bind one target at a time or bind two targets simultaneously. In some embodiments, a binding peptide is a cysteine motif binding peptide

### Tyrosinase Polypeptides

Tyrosinase polypeptides that are suitable for use in generating a reactive moiety (e.g., an orthoquinone) include a tyrosinase polypeptide having at least 75%, at least 80%, at least 85%, at least 90%, at least 95%, at least 98%, at least 99%, or 100%, amino acid sequence identity to any one of the tyrosinase amino acid sequences set forth any one of SEQ ID NOs: 1-6 and 52-53. In some embodiments, the tyrosinase polypeptide is an Agricus bisporus tyrosinase polypeptide. In some embodiments, the tyrosinase polypeptide is a Bacillus megaterium tyrosinase polypeptide. In some embodiments, the tyrosinase polypeptide is a Streptomyces castaneoglobisporus tyrosinase polypeptide. In some embodiments, the tyrosinase polypeptide is a Citrobacter freundii tyrosinase polypeptide. In some embodiments, the tyrosinase polypeptide is a Homo sapiens tyrosinase polypeptide. In some embodiments, the tyrosinase polypeptide is a Malus domestica tyrosinase polypeptide. In some embodiments, the tyrosinase polypeptide is an Aspergillus oryzae tyrosinase polypeptide. In some embodiments, the tyrosinase polypeptide is a Solanum lycopersicum tyrosinase polypeptide. In some embodiments, the tyrosinase polypeptide is a Burkholderia thailandensis tyrosinase polypeptide. In some embodiments, the tyrosinase polypeptide is a Juglans regia tyrosinase polypeptide. See, e.g., Pretzler et al. Sci. Rep. 2017, 7 (1), 1810; Ren et al. BMC Biotechnol. 2013, 13, 18; Faccio et al. Process Biochem. 2012, 47 (12), 1749-1760; Fairhead et al. FEBS J. 2010, 277 (9), 2083-2095; Do et al. Sci. Rep. 2017, 7 (1), 17267; Elsayed and Danial J. Appl. Pharm. Sci. 2018, 8 (09), 93-101; Lopez-Tejedor and Palomo Protein Expr. Purif. 2018, 145, 64-70; and Fairhead et al. Nature Biotechnol. 2012, 29 (2), 183-191.

In some embodiments, the tyrosinase polypeptide selectively acts on (e.g., generates a reactive moiety such as an orthoquinone) a substrate (a biomolecule or polypeptide) comprising a phenol moiety (e.g., a tyrosine) or a catechol moiety, where the substrate is neutral or positively charged within 50 Å (e.g., within 50 Å, within 40 Å, within 30 Å, or within 20 Å) of the phenol or the catechol moiety. For example, a tyrosinase having at least 75%, at least 80%, at least 85%, at least 90%, at least 95%, at least 98%, at least 99%, or 100%, amino acid sequence identity to any one of the tyrosinase amino acid sequences set forth in any one of SEQ ID NOs: 2 and 4-6 can selectively modify a phenol or catechol moiety on a substrate, where the substrate is neutral or positively charged within 50 Å (e.g., within 50 Å, within 40 Å, within 30 Å, or within 20 Å) of the phenol or the catechol moiety. In some embodiments, at least one of the at least two polypeptides of a conjugate comprises at least 2 neutral or positively charged amino acids within 10 amino acids of the phenol moiety (e.g., a tyrosine) or a catechol moiety. In some embodiments, at least one of the at least two polypeptides of a conjugate comprises 2, 3, 4, 5, 6, 7, 8, 9, or 10 neutral or positively charged amino acids within 10 amino acids of the phenol moiety (e.g., a tyrosine) or a catechol moiety. In some embodiments, at least one of the at least two polypeptides of a conjugate comprises the amino acid sequence GGGGCY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

In some embodiments, the tyrosinase polypeptide selectively acts on (e.g., generates a reactive moiety such as an orthoquinone) a substrate (a biomolecule) comprising a phenol moiety (e.g., a tyrosine) or a catechol moiety, where the substrate is negatively charged within 50 Å (e.g., within 50 Å, within 40 Å, within 30 Å, or within 20 Å) of the phenol or the catechol moiety. In some embodiments, a tyrosinase having at least 75%, at least 80%, at least 85%, at least 90%, at least 95%, at least 98%, at least 99%, or 100%, amino acid sequence identity to any one of the tyrosinase amino acid sequences set forth in any one of SEQ ID NOs: 2-3 can selectively modify a phenol or catechol moiety on a substrate, where the substrate is negatively charged within 50 Å (e.g., within 50 Å, within 40 Å, within 30 Å, or within 20 Å) of the phenol or the catechol moiety. In some embodiments, at least one of the at least two polypeptides of a conjugate comprises at least 2 negatively charged amino acids within 10 amino acids of the phenol moiety (e.g., a tyrosine) or a catechol moiety. In some embodiments, at least one of the at least two polypeptides of a conjugate comprises 2, 3, 4, 5, 6, 7, 8, 9, or 10 negatively charged amino acids within 10 amino acids of the phenol moiety (e.g., a tyrosine) or a catechol moiety. In some embodiments, at least one of the at least two polypeptides of a conjugate comprises the amino acid sequence GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, SEY, ESY, SRY, SKY, SNY, ERY, EKY, EGY, SEYP, SHRKY, SARKY, SPPEY, SSEEY, SEEEY, and SSSSY.

### Methods

Described herein, in certain embodiments, are methods of covalently linking at least two polypeptides to at least two payloads. In some embodiments, the methods comprise contacting a first polypeptide of the at least two polypeptides and a first payload of the at least two payloads using a first tyrosinase, wherein the first polypeptide comprises a first terminal tag comprising (X₁)ₘ₁X₂X₃X₄X₅, wherein X₁-X₅ is any amino a acid provided that at least one amino acid of X₁-X₅ is a tyrosine, and wherein m1 is an integer greater than or equal to 0; and contacting a second polypeptide of the at least two polypeptides and a second payload of the at least two payloads using a second tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0. In some embodiments, the methods comprise contacting a first polypeptide of the at least two polypeptides and a first payload of the at least two payloads using a first tyrosinase, wherein the first polypeptide comprises a first terminal tag comprising (X₁)ₘ₁X₂X₃X₄X₅, wherein X₁-X₅ is any amino a acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine; and contacting a second polypeptide of the at least two polypeptides and a second payload of the at least two payloads using a second tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0. The contacting is carried out under conditions sufficient for conjugation of the at least two payloads to the at least two polypeptides, thereby producing a conjugate.

In some embodiments, a subject method for chemoselective modification of a payload comprises contacting: i) a payload comprising a thiol moiety; ii) a polypeptide comprising a phenol moiety or a catechol moiety; and iii) an enzyme capable of oxidizing the phenol or catechol moiety; wherein the enzyme oxidizes the phenol or catechol moiety of the polypeptide to generate a reactive moiety, thereby generating a polypeptide comprising the reactive moiety, and wherein the reactive moiety reacts with the thiol moiety, thereby conjugating the payload and the polypeptide to one another, thereby producing a conjugate. In some embodiments, the payload comprises a single thiol moiety.

In some embodiments, a subject method for chemoselective modification of a polypeptide comprises contacting: i) a payload comprising a phenol moiety or a catechol moiety; ii) a polypeptide comprising a thiol moiety; and iii) an enzyme capable of oxidizing the phenol or catechol moiety; wherein the enzyme oxidizes the phenol or catechol moiety of the payload to generate a reactive moiety, thereby generating a payload comprising the reactive moiety, and wherein the reactive moiety reacts with the thiol moiety, thereby conjugating the payload and the polypeptide to one another, thereby producing a conjugate. In some embodiments, the polypeptide comprises a single thiol moiety. In some embodiments, the polypeptide comprises a two or more thiol moieties. In some embodiments, the polypeptide is modified to comprises a terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine.

In some embodiments, a subject method for chemoselective modification of a polypeptide comprises contacting: i) a payload comprising a maleimide moiety; ii) a polypeptide comprising a thiol moiety; and iii) an enzyme capable of oxidizing the maleimide moiety; wherein the enzyme oxidizes the maleimide moiety of the payload to generate a reactive moiety, thereby generating a payload comprising the reactive moiety, and wherein the reactive moiety reacts with the thiol moiety, thereby conjugating the payload and the polypeptide to one another, thereby producing a conjugate. In some embodiments, the polypeptide comprises a single thiol moiety. In some embodiments, the polypeptide comprises a two or more thiol moieties.

In some embodiments, the present disclosure provides for attachment a payload comprising a thiol moiety to a polypeptide comprising a phenol or catechol moiety. In some embodiments, the present disclosure provides for attachment for attachment of a payload comprising a phenol or catechol moiety to a polypeptide comprising a thiol moiety. In some embodiments, the present disclosure provides for attachment for attachment of a payload comprising maleimide moiety to a polypeptide comprising a thiol moiety. In some embodiments, the methods generally involve reacting thiol containing payloads with a polypeptide comprising a reactive moiety (e.g., an orthoquinone moiety). In some embodiments, the methods generally involve reacting thiol containing polypeptides with at least one payload comprising a reactive moiety (e.g., an orthoquinone moiety). In some embodiments, the methods generally involve reacting thiol containing polypeptides with at least one payload comprising a maleimide moiety.

The methods disclosed herein provide a simple coupling procedure that can attach payloads of interest in a site-specific manner to any position on the surface of polypeptides, thereby producing a conjugate of interest. The payload can be any of a variety of molecules (e.g., polypeptides; nucleic acids; small molecules; etc.). In some embodiments, the payload is a small molecule (e.g., a cancer chemotherapeutic agent).

Polypeptides of interest include antibodies. In some instances, the polypeptide of interest is an antibody fragment or binding derivative thereof. In some embodiments, the antibody fragment or binding derivative thereof is selected from the group consisting of a Fab fragment, a F(ab′)2 fragment, a single-chain Fv (scFv), a diabody, a nanobody, and a triabody. In some embodiments, the antibody fragment or binding derivative thereof is selected from the group consisting of a heavy chain variable region (VH), a light chain variable region (VL), a heavy chain, a light chain, or a constant chain of an antibody.

In some embodiments, a polypeptide further comprises one or more moieties selected from a fluorophore, an active small molecule, an affinity tag, and a metal-chelating agent. In some embodiments, the polypeptide further comprises a fluorescent protein. In some embodiments, the fluorescent protein is a green fluorescent protein (GFP). In some embodiments, the polypeptide is an enzyme. In some embodiments, the polypeptide is a receptor.

Payloads of interest include, but are not limited to, small molecules, polypeptides, polynucleotides, nucleic acids, carbohydrates, lipids, fatty acids, steroids, purines, pyrimidines, derivatives, structural analogs thereof and combinations thereof. In some embodiments, the payload is a small molecule (e.g., a cancer chemotherapeutic agent); and the polypeptide is an antibody (e.g., a scFv; a nanobody; and the like).

In some embodiments, the enzyme capable of oxidizing the phenol moiety or the catechol moiety is a phenol oxidase or a catechol oxidase. In certain cases, the enzyme is a tyrosinase.

The term “tyrosinase” is used herein to refer to monophenol monooxygenase (EC 1.14.18.1; CAS number: 9002-10-2) or a derivative thereof, an enzyme that catalyzes the oxidation of phenols (such as tyrosine). They are copper-containing enzymes originally present in plant and animal tissues that catalyzes the production of melanin and other pigments from tyrosine by oxidation. Tyrosinases have in common a binuclear type 3 copper center within their active site. Here two copper atoms are each coordinated with three histidine residues. Matoba et al., “Crystallographic evidence that the dinuclear copper center of tyrosinase is flexible during catalysis,” J Biol Chem. 2006 Mar. 31; 281(13):8981-90. Epub 2006 Jan. 25, disclose a three dimensional model of a tyrosinase catalytic center.

In some embodiments, the phenol moiety of a conjugate is present in a tyrosine residue. In some embodiments, the tyrosine residue is part of the polypeptide of interest. In some embodiments, the tyrosine residue is synthetically introduced into a polypeptide of a conjugate. In some embodiments, the tyrosine residue is linked to a polypeptide via a linker (e.g., as described herein). A tyrosine residue can be introduced using standard recombinant techniques, e.g., by modifying a nucleotide sequence encoding a polypeptide such that a tyrosine residue is introduced into the polypeptide.

In some embodiments, a phenol or catechol moiety is part of an unnatural (non-genetically encoded) amino acid that is introduced into a polypeptide of interest. For example, amber codon (TAG) suppression can be used to incorporate a non-genetically encoded amino acid residue that comprises a phenol moiety or a catechol moiety. See, e.g., Chin et al. (2002) J. Am. Chem. Soc. 124:9026; Chin and Schultz (2002) Chem. Biol. Chem. 3:1135; Chin et al. (2002) Proc. Natl. Acad. Sci. USA 99:11020; U.S. 2015/0240249; and US 2018/0171321. As another example, an orthogonal RNA synthetase and/or an orthogonal tRNA can be used for introducing a non-genetically encoded amino acid into a polypeptide, where the non-genetically encoded amino acid comprises a phenol moiety or a catechol moiety.

In some embodiments of the subject methods, the thiol moiety present in the payload is part of a cysteine residue. In certain cases, the cysteine residue is a native cysteine residue. In certain cases, the cysteine residue is a residue synthetically introduced into the target molecule.

In some embodiments, a polypeptide contains a thiol moiety and the payload contains a phenol or catechol moiety.

In certain embodiments, the reactive moiety is an orthoquinone or a semi-quinone radical, or a combination thereof. In certain embodiments, the subject methods provide a reaction between an orthoquinone reactive intermediate and a thiol moiety, as depicted in Scheme 1 below:

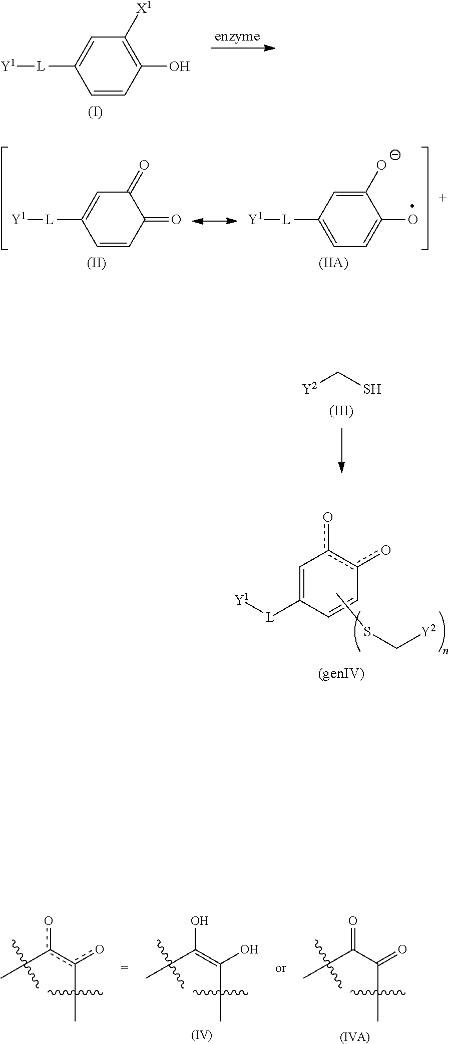

where Y¹ is a polypeptide; L is an optional linker (e.g., as described herein); X¹ is selected from hydrogen and hydroxyl; Y² is a payload; and n is an integer from 1 to 3.

As depicted in Scheme 1, in certain embodiments, a polypeptide comprising a phenol or catechol moiety (e.g., of formula (I)), undergoes activation with an enzyme capable of oxidizing the phenol or catechol moiety. In some embodiments, activation is achieved with a tyrosinase enzyme in the presence of oxygen to generate an intermediate comprising a reactive moiety (e.g., orthoquinone of formula (II) and/or semi-quinone radical of formula (IIA)), and the said reactive moiety reacts with a payload comprising a thiol based nucleophile (e.g., of formula (III)), to result in conjugation of the target molecule to the biomolecule, thereby producing a modified target molecule (e.g., of formula (genIV)). In certain embodiments, a payload of formula (III) may comprise any payload, e.g., as described herein. In some embodiments, Y2 in formula (III) is a polypeptide. In some embodiments, the conjugate is described by the formula (IV). In some embodiments, the conjugate is described by the formula (IV A).

In certain embodiments, the subject methods provide a reaction between an orthoquinone reactive intermediate and a thiol moiety, as depicted in Scheme 2 below:

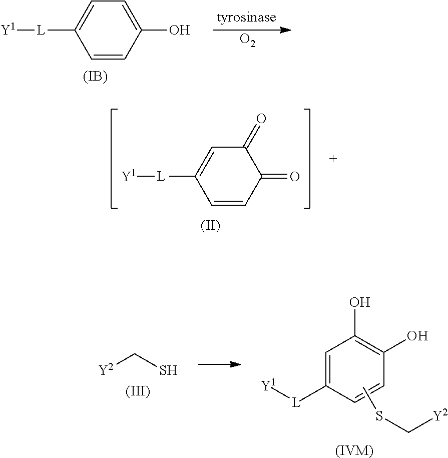

As depicted in Scheme 2, in certain embodiments, a polypeptide comprising a phenol moiety (e.g., of formula (IB)) undergoes activation with a tyrosinase enzyme in the presence of oxygen to generate an intermediate comprising a reactive moiety (e.g., orthoquinone of formula (II)), and the said reactive moiety reacts with a payload comprising a thiol based nucleophile (e.g., of formula (III)), to result in conjugation of the target molecule to the biomolecule, thereby producing a modified target molecule (e.g., of formula (IVM). In certain embodiments, a payload of formula (III) may comprise any payload, e.g., as described herein. In certain cases, Y² in formula (III) is a polypeptide. In certain cases of the conjugate of formula (IVM), the thiol group is at the 3-position of the catechol ring. In certain cases of the conjugate of formula (IVM), the thiol group is at the 5-position of the catechol ring. In certain cases of the conjugate of formula (IVM), the thiol group is at the 6-position of the catechol ring.

In certain embodiments, the subject methods provide a reaction between an orthoquinone reactive intermediate and an amine moiety (e.g., a lysine residue present in polypeptide), as depicted in Scheme 3 below:

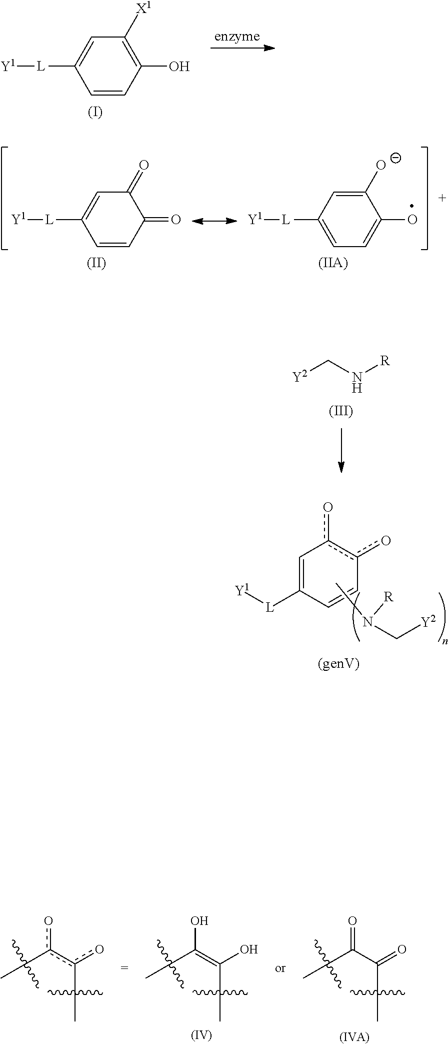

where Y¹ is a polypeptide; L is an optional linker (e.g., as described herein); X¹ is selected from hydrogen and hydroxyl; Y² is a payload; R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and n is an integer from 1 to 3. In certain instances, R is an amino acid selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine.

As depicted in Scheme 3, in certain embodiments, a polypeptide comprising a phenol or catechol moiety (e.g., of formula (I)), undergoes activation with an enzyme capable of oxidizing the phenol or catechol moiety. In some embodiments, activation is achieved with a tyrosinase enzyme in the presence of oxygen to generate an intermediate comprising a reactive moiety (e.g., orthoquinone of formula (II) and/or semi-quinone radical of formula (IIA)), and the said reactive moiety reacts with a payload comprising an amine based nucleophile (e.g., of formula (III)), to result in conjugation of the target molecule to the biomolecule, thereby producing a modified target molecule (e.g., of formula (genIV)). In certain embodiments, a payload of formula (III) may comprise any payload, e.g., as described herein. In some embodiments, Y2 in formula (III) is a polypeptide. In some embodiments, the conjugate is described by the formula (IV). In some embodiments, the conjugate is described by the formula (IV A).

In certain embodiments, the subject methods provide a reaction between an orthoquinone reactive intermediate and an imidazole moiety (e.g., a histidine residue present in a polypeptide), as depicted in Scheme 4 below:

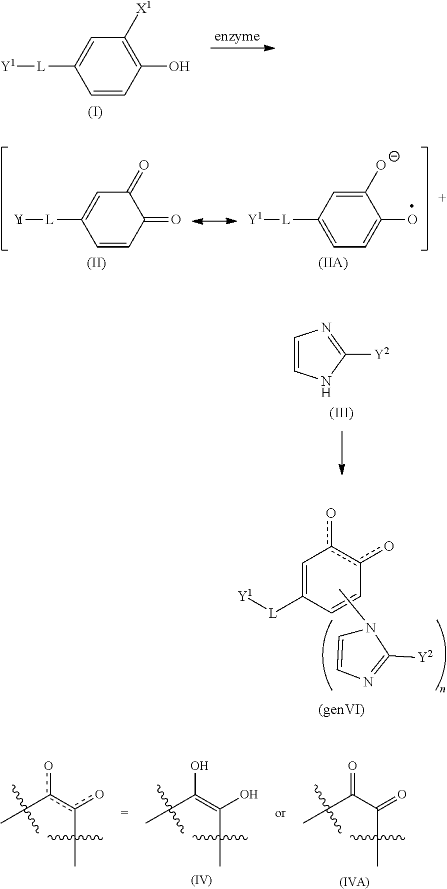

where Y¹ is a polypeptide; L is an optional linker (e.g., as described herein); X¹ is selected from hydrogen and hydroxyl; Y² is a payload; and n is an integer from 1 to 3.

As depicted in Scheme 4, in certain embodiments, a polypeptide comprising a phenol or catechol moiety (e.g., of formula (I)), undergoes activation with an enzyme capable of oxidizing the phenol or catechol moiety. In some embodiments, activation is achieved with a tyrosinase enzyme in the presence of oxygen to generate an intermediate comprising a reactive moiety (e.g., orthoquinone of formula (II) and/or semi-quinone radical of formula (IIA)), and the said reactive moiety reacts with a payload comprising an imidazole based nucleophile (e.g., of formula (III)), to result in conjugation of the target molecule to the biomolecule, thereby producing a modified target molecule (e.g., of formula (genIV)). In certain embodiments, a payload of formula (III) may comprise any payload, e.g., as described herein. In some embodiments, Y2 in formula (III) is a polypeptide. In some embodiments, the conjugate is described by the formula (IV). In some embodiments, the conjugate is described by the formula (IV A).

In certain embodiments, the subject methods provide a reaction between an orthoquinone reactive intermediate and an amine moiety, as depicted in Scheme 4 below:

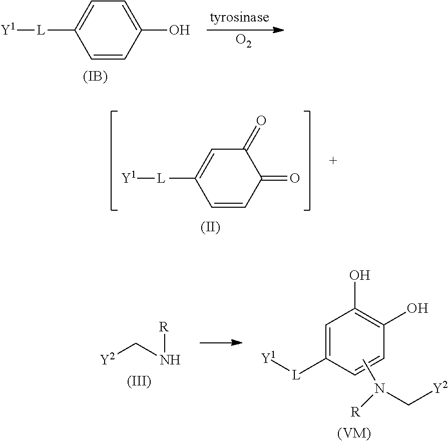

In certain embodiments, the subject methods provide a reaction between an orthoquinone reactive intermediate and an imidazole moiety, as depicted in Scheme 5 below:

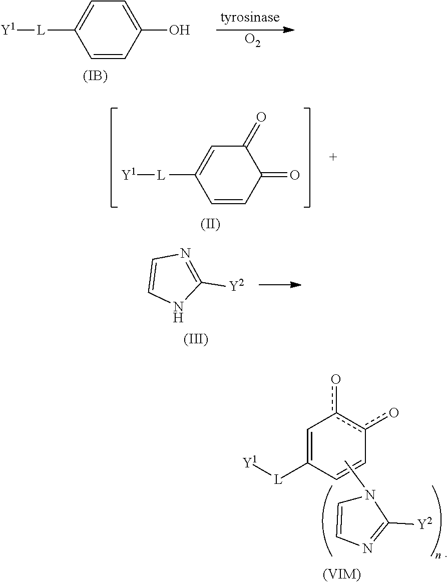

In some embodiments, the method is carried out at a pH from 4 to 9, such as 4.2, 4.5, 4.8, 5.0, 5.2, 5.5, 5.8, 6.0, 6.2, 6.5, 6.8, 7.0, 7.2, 7.5, 7.8, 8.0, 8.2, 8.5, 8.8 or 9. In some embodiments, the method is carried out at a pH of from 5 to 8, such as 5.2, 5.5, 5.8, 6.0, 6.2, 6.5, 6.8, 7.0, 7.2, 7.5, 7.8 or 8.0. In certain cases, the method is carried out at a pH of 6 to 7.5, such as 6.0, 6.3, 6.4, 6.5, 6.6, 6.8, 7.0, 7.2, 7.4, or 7.5. In some embodiments, the method is carried out at neutral pH. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In some embodiments, the methods may be carried out under physiological conditions. In some embodiments, the method is carried out on living cells in vitro. In other embodiments, the method is carried out on living cells ex vivo.

In some embodiments, the methods may be carried out in aqueous media in the presence of one or more buffers. Buffers of interest include, but are not limited to, a phosphate buffer, 2-amino-2-(hydroxymethyl)propane-1,3-diol (TRIS), 4-[4-(2-hydroxyethyl)piperzin-1-yljethanesulfonic acid (HEPES), and the like. In some embodiments, the methods may be carried out in an organic solvent. In some embodiments, the organic solvent is a water miscible solvent. In some embodiments, the organic solvent is a dipolar aprotic solvent. In some embodiments, the organic solvent is selected from acetonitrile, dimethyl formamide, methanol and acetone. In certain cases, the organic solvent is present an amount from 1 to 20%, relative to water, such as 2%, 5%, 10%, 15% or 20%. In some embodiments, the subject method is carried out in from 1% to 20% acetonitrile, such as 5%, 10%, 15% or 20%. In some embodiments, the subject method is carried out in from 1% to 20% dimethyl formamide, such as 5%, 10%, 15%, or 20%. In some embodiments, the subject method is carried out in from 1% to 20% methanol, such as 5%, 10%, 15%, or 20%. In some embodiments, the subject method is carried out in from 1% to 20% acetone, such as 5%, 10%, 15%, or 20%.

In some embodiments, the buffer can be 50 mM Phosphate pH 6.5, 150 mM NaCl, and 2 mM EDTA or 50 mM Acetate pH 5.5, 10% sucrose 1 mM EDTA. The phosphate in the first listed buffer can be replaced with HEPES, Tris, Acetate, and MES buffers, from pH 5 to pH 9. In some embodiments, sucrose and glucose can be added to the buffer up to 15% v/v, Trehalose can be added at up to 300 mM, and EDTA can be added at up to 5 mM.

In some embodiments, the conjugate is a product of double or triple conjugation (e.g., referring to formula (IV), when n is 2 or 3, referred to collectively herein as “multiple conjugation products”). In some embodiments, multiple conjugation products are present in less than 1 part in 10 by weight of one or more multiple conjugation products relative to the single conjugation product (e.g., referring to formula (IV), when n is 1), such as less than 1 part in 20, less than 1 part in 25, less than 1 part in 50, less than 1 part in 75, less than 1 part in 100, or even less. In some embodiments, no multiple conjugation products are observed.

In some embodiments, the conjugate is stable at a range of pH and temperature values and in the presence of a number of additional molecules. In some embodiments, the conjugate is stable from 0° C. to 50° C., such as 4° C. to 40° C., such as 4° C. to 37° C. In certain cases, the conjugate is stable over a pH range of 4 to 9, such as at pH 4, 4.5, 5, 5.5, 6, 6.5, 7, 7.5, 8, 8.5 or 9. In some embodiments, the conjugate is stable in the presence of biologically relevant molecules. In some embodiments, the conjugate is stable in the presence of molecules such as, the guanidinium group of an arginine residue, the primary amine of a lysine residue, and aniline moieties. In some embodiments, the conjugate is stable in physiological conditions; for example, In some embodiments, the conjugate is stable in human serum. In some embodiments, the conjugate is stable in human serum at 37° C. for a period of time of at least 2 days, at least 3 days, at least 4 days, at least 5 days, at least 6 days, at least 7 days, at least 10 days, or at least 14 days. In some embodiments, the conjugate is stable in human serum at 37° C. for a period of time from about 2 days to about 7 days, from about 7 days to about 10 days, or from about 10 days to about 14 days.

The present disclosure provides a method of linking at least two polypeptides to at least two payloads in a sequential manner. The method takes advantage of the substrate preferences of tyrosinase polypeptides, as described above. The method can be carried out on an insoluble substrate, i.e., an immobilized surface, such as a bead.

Thus, the present disclosure provides a method of linking at least two polypeptides to at least two payloads, the method comprising: a) contacting a first polypeptide of the at least two polypeptides and a first payload of the at least two payloads using a first tyrosinase, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0; and b) contacting a second polypeptide of the at least two polypeptides and a second payload of the at least two payloads using a second tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0. In some embodiments, the present disclosure provides a method of linking at least two polypeptides to at least two payloads, the method comprising: a) contacting a first polypeptide of the at least two polypeptides and a first payload of the at least two payloads using a first tyrosinase, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine, and wherein m1 is an integer greater than or equal to 0, or X₁X₂X₃, wherein X₁-X₃ is any amino acid provided that at least one amino acid of X₁-X₃ is a tyrosine; and b) contacting a second polypeptide of the at least two polypeptides and a second payload of the at least two payloads using a second tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine, and wherein m2 is an integer greater than or equal to 0. In some embodiments, the first polypeptide comprises two or more negatively charged amino acids within ten amino acids of the phenol moiety or the catechol moiety and the second polypeptide comprises two or more neutral or positively charged within ten amino acids of the phenol moiety or the catechol moiety. In some embodiments, the first enzyme is a tyrosinase polypeptide comprising an amino acid sequence having at least 75% amino acid sequence identity to any one of the amino acid sequences of SEQ ID NOs: 1-6, 52, or 53. In some embodiments, the second enzyme is a tyrosinase polypeptide comprising an amino acid sequence having at least 80% amino acid sequence identity to any one of the amino acid of SEQ ID NOs: 1-6, 52, or 53.

In some embodiments, the first and/or second enzyme is a tyrosinase polypeptide comprising an amino acid sequence having at least 85% amino acid sequence identity to any one of the amino acid sequences of SEQ ID NOs: 1-6, 52, or 53. In some embodiments, the first and/or second enzyme is a tyrosinase polypeptide comprising an amino acid sequence having at least 90% amino acid sequence identity to any one of the amino acid of SEQ ID NOs: 1-6, 52, or 53. In some embodiments, first and/or second enzyme is a tyrosinase polypeptide comprising an amino acid sequence having at least 95% amino acid sequence identity to any one of the amino acid sequences of SEQ ID NOs: 1-6, 52, or 53. In some embodiments, the first and/or second enzyme is a tyrosinase polypeptide comprising an amino acid sequence having at least 99% amino acid sequence identity to any one of the amino acid sequences of SEQ ID NOs: 1-6, 52, or 53. In some embodiments, the first and/or second enzyme is a tyrosinase polypeptide comprising an amino acid sequence according to any one of the amino acid sequences of SEQ ID NOs: 1-6, 52, or 53.

In some embodiments, the first and/or second enzyme is a tyrosinase polypeptide comprising an amino acid sequence having at least 85% amino acid sequence identity to any one of the amino acid sequences of SEQ ID NOs: 2-6. In some embodiments, the first and/or second enzyme is a tyrosinase polypeptide comprising an amino acid sequence having at least 90% amino acid sequence identity to any one of the amino acid sequences of SEQ ID NOs: 2-6. In some embodiments, the first and/or second enzyme is a tyrosinase polypeptide comprising an amino acid sequence having at least 95% amino acid sequence identity to any one of the amino acid sequences of SEQ ID NOs: 2-6. In some embodiments, the first and/or second enzyme is a tyrosinase polypeptide comprising an amino acid sequence having at least 99% amino acid sequence identity to any one of the amino acid sequences of SEQ ID NOs: 2-6. In some embodiments, the second enzyme is a tyrosinase polypeptide comprising an amino acid sequence according to any one of the amino acid sequences of SEQ ID NOs: 2-6.

In some embodiments, the first and/or second enzyme is a tyrosinase polypeptide comprising an amino acid sequence having at least 85% amino acid sequence identity to the amino acid sequence of SEQ ID NO: 2. In some embodiments, the first and/or second enzyme is a tyrosinase polypeptide comprising an amino acid sequence having at least 90% amino acid sequence identity to the amino acid sequence of SEQ ID NO: 2. In some embodiments, the first and/or second enzyme is a tyrosinase polypeptide comprising an amino acid sequence having at least 95% amino acid sequence identity to the amino acid sequence of SEQ ID NO: 2. In some embodiments, the first and/or second enzyme is a tyrosinase polypeptide comprising an amino acid sequence having at least 99% amino acid sequence identity to the amino acid sequence of SEQ ID NO: 2. In some embodiments, the first and/or second enzyme is a tyrosinase polypeptide comprising an amino acid sequence according to amino acid of SEQ ID NO: 2.

In some embodiments, the first and/or second enzyme is a tyrosinase polypeptide comprising an amino acid sequence having at least 85% amino acid sequence identity to the amino acid sequence of SEQ ID NO: 3. In some embodiments, the first and/or second enzyme is a tyrosinase polypeptide comprising an amino acid sequence having at least 90% amino acid sequence identity to the amino acid sequence of SEQ ID NO: 3. In some embodiments, the first and/or second enzyme is a tyrosinase polypeptide comprising an amino acid sequence having at least 95% amino acid sequence identity to the amino acid sequence of SEQ ID NO: 3. In some embodiments, the first and/or second enzyme is a tyrosinase polypeptide comprising an amino acid sequence having at least 99% amino acid sequence identity to the amino acid sequence of SEQ ID NO: 3. In some embodiments, the first and/or second enzyme is a tyrosinase polypeptide comprising an amino acid sequence according to amino acid of SEQ ID NO: 3.

In some embodiments, the first and/or second enzyme is a tyrosinase polypeptide comprising an amino acid sequence having at least 85% amino acid sequence identity to the amino acid sequence of SEQ ID NO: 4. In some embodiments, the first and/or second enzyme is a tyrosinase polypeptide comprising an amino acid sequence having at least 90% amino acid sequence identity to the amino acid sequence of SEQ ID NO: 4. In some embodiments, the first and/or second enzyme is a tyrosinase polypeptide comprising an amino acid sequence having at least 95% amino acid sequence identity to the amino acid sequence of SEQ ID NO: 4. In some embodiments, the first and/or second enzyme is a tyrosinase polypeptide comprising an amino acid sequence having at least 99% amino acid sequence identity to the amino acid sequence of SEQ ID NO: 4. In some embodiments, the first and/or second enzyme is a tyrosinase polypeptide comprising an amino acid sequence according to amino acid of SEQ ID NO: 4.

In some embodiments, the first and/or second enzyme is a tyrosinase polypeptide comprising an amino acid sequence having at least 85% amino acid sequence identity to the amino acid sequence of SEQ ID NO: 5. In some embodiments, the first and/or second enzyme is a tyrosinase polypeptide comprising an amino acid sequence having at least 90% amino acid sequence identity to the amino acid sequence of SEQ ID NO: 5 In some embodiments, the first and/or second enzyme is a tyrosinase polypeptide comprising an amino acid sequence having at least 95% amino acid sequence identity to the amino acid sequence of SEQ ID NO: 5. In some embodiments, the first and/or second enzyme is a tyrosinase polypeptide comprising an amino acid sequence having at least 99% amino acid sequence identity to the amino acid sequence of SEQ ID NO: 5. In some embodiments, the first and/or second enzyme is a tyrosinase polypeptide comprising an amino acid sequence according to amino acid of SEQ ID NO: 5.

In some embodiments, the first and/or second enzyme is a tyrosinase polypeptide comprising an amino acid sequence having at least 85% amino acid sequence identity to the amino acid sequence of SEQ ID NO: 6. In some embodiments, the first and/or second enzyme is a tyrosinase polypeptide comprising an amino acid sequence having at least 90% amino acid sequence identity to the amino acid sequence of SEQ ID NO: 6. In some embodiments, the first and/or second enzyme is a tyrosinase polypeptide comprising an amino acid sequence having at least 95% amino acid sequence identity to the amino acid sequence of SEQ ID NO: 6. In some embodiments, the first and/or second enzyme is a tyrosinase polypeptide comprising an amino acid sequence having at least 99% amino acid sequence identity to the amino acid sequence of SEQ ID NO: 4. In some embodiments, the first and/or second enzyme is a tyrosinase polypeptide comprising an amino acid sequence according to amino acid of SEQ ID NO: 6.

In some embodiments, the phenol moiety present in the first polypeptide is present in a Tyrosine (Tyr, Y) residue. In some embodiments, the phenol moiety present in the second polypeptide is present in a Tyr residue.

By alternating use of: a) a tyrosinase enzyme that preferentially modifies a Tyr residue that is present in a negatively charged environment (e.g., where the polypeptide comprises two or more negatively charged within ten amino acids of the Tyr residue); and b) a tyrosinase enzyme that preferentially modifies a Tyr residue that is present in a neutral or positively charged environment ((e.g., where the polypeptide comprises two or more neutral or positively charged within ten amino acids of the Tyr residue), a first payload can be added to the first polypeptide and a second payload can be added to the second polypeptide in a sequential manner. In some embodiments, the first payload and the second payload are the same. In some embodiments, the first payload and the second payload are different.

In some embodiments, the tyrosinase enzyme is inactivated or removed between any two steps of the method and before adding a further tyrosinase enzyme. For example, between step (b) and step (c) of the method described above, the second enzyme is inactivated or removed.

Described herein, in certain embodiments, are methods for selectively conjugating at least two polypeptides to at least two payloads by modulating the temperature during the one or more tyrosinase reactions. In some embodiments, a first polypeptide (e.g., a heavy chain) is selectively modified through temperature control during a first tyrosinase reaction. In some embodiments, the temperature during the first tyrosinase reaction occurs at a temperature of about 0° C. to about 18° C., such as about 0° C. to 15° C., about 0° C. to 10° C., about 4° C. to 15° C., or about 4° C. to 10° C. In some embodiments, the temperature during the first tyrosinase reaction occurs at a temperature of about 4° C. In some embodiments, the temperature during the first tyrosinase reaction occurs at room temperature. In some embodiments, a second polypeptide (e.g., a light chain) is selectively modified through temperature control during a second tyrosinase reaction. In some embodiments, the temperature during the second tyrosinase reaction occurs at a temperature of about 23° C. to about 50° C., such as about 25° C. to 50° C., about 25° C. to 40° C., or about 25° C. to 37° C. In some embodiments, the temperature during the second tyrosinase reaction occurs at a temperature of about 30° C. In some embodiments, the temperature during the second tyrosinase reaction occurs at a temperature of about 37° C. In some embodiments, the temperature during the second tyrosinase reaction occurs at room temperature. In some embodiments, the same tyrosinase is used for the reaction at the different temperatures.

Described herein, in certain embodiments, are methods for selectively conjugating at least two polypeptides to at least two payloads by deglycosylation of one or more polypeptides of the least two polypeptides. In some embodiments, one or more sugars of the one or more polypeptides is removed (i.e., deglycosylated). In some embodiments, at least about 5%, 10%, 15%, 20%, 25%, 30%, 35%, 40%, 45%, 50%, 55%, 60%, 65%, 70%, 75%, 80%, 85%, 90%, or more than 90% of the sugars of the one or more polypeptides is removed. In some embodiments, removal of the one or more sugars occurs at a temperature of about 0° C. to about 50° C., such as about 4° C. to 40° C. or about 4° C. to 37° C. In some embodiments, removal of the one or more sugars occurs at a temperature of about 4° C. In some embodiments, removal of the one or more sugars occurs at a temperature of about 37° C. In some embodiments, the deglycosylated antibodies are conjugated to one or more payloads at a temperature of about 4° C. In some embodiments, the deglycosylated antibodies are conjugated to one or more payloads at a temperature of about 37° C. In some embodiments, the deglycosylated antibodies are conjugated to one or more payloads at a temperature of about room temperature.

Described herein, in certain embodiments, are methods for selectively conjugating at least two polypeptides to at least two payloads by aglycosylation of one or more polypeptides of the least two polypeptides. In some embodiments, aglycosylation is achieved by incorporating mutations in the polypeptide to remove the glycosylation site. In some embodiments, the mutation include changing an asparagine to an alanine. In some embodiments, the mutation include changing an asparagine to a glutamine. In some embodiments, the mutation include changing an asparagine to a glycine. In some embodiments, the mutation include changing a serine to a glycine. In some embodiments, the mutation include changing a serine to an alanine. In some embodiments, the mutation include changing a threonine to an alanine. In some embodiments, one or more sugars of the one or more polypeptides is removed by mutations (i.e., aglycosylated). A person of skill in the art would know how to use routine methods to incorporate the proper mutation into the coding sequence of the polypeptide to achieve the intended mutation. In some embodiments, at least about 5%, 10%, 15%, 20%, 25%, 30%, 35%, 40%, 45%, 50%, 55%, 60%, 65%, 70%, 75%, 80%, 85%, 90%, or more than 90% of the sugars of the one or more polypeptides is removed. In some embodiments, the aglycosylated antibodies are conjugated to one or more payloads at a temperature of about 4° C. In some embodiments, the aglycosylated antibodies are conjugated to one or more payloads at a temperature of about 37° C. In some embodiments, the aglycosylated antibodies are conjugated to one or more payloads at a temperature of about 30° C.

Described herein, in certain embodiments, are methods for selectively conjugating at least two polypeptides to at least two payloads, wherein conjugation occurs at less than about 10 hours, 9 hours, 8 hours, 7 hours, 6 hours, 5 hours, 4 hours, 3 hours, 2 hours, or 1 hour. In some embodiments, the conjugation occurs less than about 1 hour, 50 minutes, 40 minutes, 30 minutes, 20 minutes, 10 minutes, 5 minutes, 1 minute, or 30 seconds. In some embodiments, the conjugation occurs less than about 10 minutes. In some embodiments, the first polypeptide is contacted with the tyrosinase for 5-70 minutes. In some embodiments, the first polypeptide is contacted with the tyrosinase for 45 min. In some embodiments, In some embodiments, In some embodiments, the first polypeptide is contacted with the tyrosinase for 60 min. In some embodiments, the second polypeptide is contacted with the tyrosinase for more than 15 minutes. In some embodiments, the second polypeptide is contacted with the tyrosinase for 60-160 minutes. In some embodiments, the second polypeptide is contacted with the tyrosinase for 90 min. In some embodiments, the second polypeptide is contacted with the tyrosinase for 120 min.

### Therapeutic Uses

The present disclosure provides methods of treating a cancer that expresses a target antigen, the method comprising administering a conjugate disclosed herein, or a pharmaceutical composition containing the conjugate, to a subject in need thereof. In some embodiments, the cancer expresses the target antigen of a polypeptide or a composition of at least two polypeptides disclosed herein.

The present disclosure provides methods of treating a cancer that expresses HER2, the method comprising a conjugate disclosed herein, or a pharmaceutical composition containing the conjugate, to a subject in need thereof. In some embodiments, the cancer is a HER2 low expressing cancer. In some embodiments, the HER2 low expressing cancer is breast cancer. In some embodiments, the cancer is a HER2 high expressing (i.e., HER2 positive) cancer. In some embodiments, the HER2 high expressing cancer is breast cancer.

In some embodiments, the method or pharmaceutical composition comprises a conjugate disclosed herein at a dose of 1 mg/kg. In some embodiments, the method or pharmaceutical composition comprises a conjugate disclosed herein at a dose of 3 mg/kg. In some embodiments, the method or pharmaceutical composition comprises a conjugate disclosed herein at a dose of 8 mg/kg.

Sequence Listing

| SEQ ID NO | Sequence |
|---|---|
| 1 | MSDKKSLMPLVGIPGEIKNRLNILDFVKNDKFFTLYVRALQVLQAR |
| Agricus bisporus | DQSDYSSFFQLGGIHGLPYTEWAKAQPQLHLYKANYCTHGTVLFP |
| tyrosinase | TWHRAYESTWEQTLWEAAGTVAQRFTTSDQAEWIQAAKDLRQPF |
| (abTYR) | WDWGYWPNDPDFIGLPDQVIRDKQVEITDYNGTKIEVENPILHYK |
| | FHPIEPTFEGDFAQWQTTMRYPDVQKQENIEGMIAGIKAAAPGFRE |
| | WTFNMLTKNYTWELFSNHGAVVGAHANSLEMVHNTVHFLIGRDP |
| | TLDPLVPGHMGSVPHAAFDPIFWMHHCNVDRLLALWQTMNYDV |
| | YVSEGMNREATMGLIPGQVLTEDSPLEPFYTKNQDPWQSDDLEDW |
| | ETLGFSYPDFDPVKGKSKEEKSVYINDWVHKHYGFVTTQTENPAL |
| | RLLSSFQRAKSDHETQYALYDWVIHATFRYYELNNSFSIIFYFDEGE |
| | GCTLESIIGTVDAFRGTTSENCANCARSQDLIAEGFVHLNYYIGCDI |
| | GQHADHEDDAVPLYEPTRVKEYLKKRKIGCKVVSAEGELTSLVVE |
| | IKGAPYYLPVGEARPKLDHEKPIVILDDIIHRVN |
| 2 | MSNKYRVRKNVLHLTDTEKRDFVRTVLILKEKGIYDRYIAWHGAA |
| megaTYR | GKFHTPPGSDRNAAHMSSAFLPWHREYLLRFERDLQSINPEVTLPY |
| (Catenase) | WEWETDAQMQDPSQSQIWSADFMGGNGNPIKDFIVDTGPFAAGR |
| | WTTIDEQGNPSGGLKRNFGATKEAPTLPTRDDVLNALKITQYDTPP |
| | WDMTSQNSFRNQLEGFINGPQLHNRVHRWVGGQMGVVPTAPNDP |
| | VFFLHHANVDRIWAVWQIIHRNQNYQPMKNGPFGQNFRDPMYPW |
| | NTTPEDVMNHRKLGYVYDIELRKSKRSSLEHHHHHH |
| 3 | MSNKYRVRKNVLHLTDTEKRDFVRTVLILKEKGIYDRYIAWHGAA |
| D55R Catenase | GKFHTPPGSRRNAAHMSSAFLPWHREYLLRFERDLQSINPEVTLPY |
| | WEWETDAQMQDPSQSQIWSADFMGGNGNPIKDFIVDTGPFAAGR |
| | WTTIDEQGNPSGGLKRNFGATKEAPTLPTRDDVLNALKITQYDTPP |
| | WDMTSQNSFRNQLEGFINGPQLHNRVHRWVGGQMGVVPTAPNDP |
| | VFFLHHANVDRIWAVWQIIHRNQNYQPMKNGPFGQNFRDPMYPW |
| | NTTPEDVMNHRKLGYVYDIELRKSKRSSLEHHHHHH |
| 4 | MSNKYRVRKNVLHLTDTEKRDFVRTVLILKEKGIYDRYIAWHGAA |
| G216E Catenase | GKFHTPPGSDRNAAHMSSAFLPWHREYLLRFERDLQSINPEVTLPY |
| | WEWETDAQMQDPSQSQIWSADFMGGNGNPIKDFIVDTGPFAAGR |
| | WTTIDEQGNPSGGLKRNFGATKEAPTLPTRDDVLNALKITQYDTPP |
| | WDMTSQNSFRNQLEGFINGPQLHNRVHRWVGGQMEVVPTAPNDP |
| | VFFLHHANVDRIWAVWQIIHRNQNYQPMKNGPFGQNFRDPMYPW |
| | NTTPEDVMNHRKLGYVYDIELRKSKRSSLEHHHHHH |
| 5 | MSNKYRVRKNVLHLTDTEKRDFVRTVLILKEKGIYDRYIAWHGAA |
| R209H Catenase | GKFHTPPGSDRNAAHMSSAFLPWHREYLLRFERDLQSINPEVTLPY |
| | WEWETDAQMQDPSQSQIWSADFMGGNGNPIKDFIVDTGPFAAGR |
| | WTTIDEQGNPSGGLKRNFGATKEAPTLPTRDDVLNALKITQYDTPP |
| | WDMTSQNSFRNQLEGFINGPQLHNRVHHWVGGQMGVVPTAPNDP |
| | VFFLHHANVDRIWAVWQIIHRNQNYQPMKNGPFGQNFRDPMYPW |
| | NTTPEDVMNHRKLGYVYDIELRKSKRSSLEHHHHHH |
| 6 | MSNKSRVRKNVLHLTDTEKRDFVRTVLILKEKGIYDRYIAWHGAA |
| Y5S Catenase | GKFHTPPGSDRNAAHMSSAFLPWHREYLLRFERDLQSINPEVTLPY |
| | WEWETDAQMQDPSQSQIWSADFMGGNGNPIKDFIVDTGPFAAGR |
| | WTTIDEQGNPSGGLKRNFGATKEAPTLPTRDDVLNALKITQYDTPP |
| | WDMTSQNSFRNQLEGFINGPQLHNRVHRWVGGQMGVVPTAPNDP |
| | VFFLHHANVDRIWAVWQIIHRNQNYQPMKNGPFGQNFRDPMYPW |
| | NTTPEDVMNHRKLGYVYDIELRKSKRSSLEHHHHHH |
| 7 | GGGGY |
| 8 | RGGGY |
| 9 | RGRGY |
| 10 | RRRGY |
| 11 | RRRRY |
| 12 | EGGGY |
| 13 | EGEGY |
| 14 | EEEGY |
| 15 | EEEEY |
| 16 | GGGWY |
| 17 | GGWGY |
| 18 | RRRWY |
| 19 | RRWRY |
| 20 | EEEWY |
| 21 | EEWEY |
| 22 | DDDDY |
| 23 | SGGY |
| 24 | KKKKY |
| 25 | RRKKY |
| 26 | RRRKY |
| 27 | EDEDY |
| 28 | EDDDY |
| 29 | EEDDY |
| 30 | RRKKY |
| 31 | KKGGY |
| 32 | DDGGY |
| 33 | EEEY |
| 34 | HHHHH |
| 35 | HHHHHHHH |
| 36 | EQKLISEEDL |
| 37 | DYKDDDDK |
| 38 | WSHPQFEK |
| 39 | YPYDVPDYA |
| 40 | RYIRS |
| 41 | FHHT |
| 42 | WEAAAREACCRECCARA |
| 43 | EGGY |
| 44 | SSEEY |
| 45 | ESSY |
| 46 | SHRKY |
| 47 | SARKY |
| 48 | SPPEY |
| 49 | SESY |
| 50 | SEEEY |
| 51 | SSSSY |
| 52 | SLLATVGPTGGVKNRLDIVDFVRDEKFFTLYIRALQAIQDKDQSDY |
| Agricus bisporus | SSFFQLSGIHGLPFTPWAKPKDTPTVPYESGYCTHSQVLFPTWHRV |
| tyrosinase | YVSIYEQILQEAAKGIAKKFTVHKKEWAQAAEDLRQPYWDTGFAL |
| abPP04 (abTYR) | VPPDEIIKLEQVKITNYDGTKITVRNPILRYSFHPIDPSFNGYPNFDT |
| | WKTTVRNPDADKKENIPALIGKLDLEADSTREKTYNMLKENANW |
| | EAFSNHGEFDDTHANSLEAVHDDIHGFVGRGAIRGHMTHALFAAF |
| | DPIFWLHHSNVDRHLSLWQALYPGVWVTQGPEREGSMGFAPGTE |
| | LNKDSALEPFYETEDKPWTSVPLTDTALLNYSYPDFDKVKGGTPD |
| | LVRDYINDHIDRRYGIKKS |
| 53 | SDKKSLMPLVGIPGEIKNRLNILDFVKNDKFFTLYVRALQVLQARD |
| Agricus bisporus | QSDYSSFFQLGGIHGLPYTEWAKAQPQLHLYKANYCTHGTVLFPT |
| tyrosinase | WHRAYESTWEQTLWEAAGTVAQRFTTSDQAEWIQAAKDLRQPF |
| abPPO3 | WDWGYWPNDPDFIGLPDQVIRDKQVEITDYNGTKIEVENPILHYKF |
| (abTYR) | HPIEPTFEGDFAQWQTTMRYPDVQKQENIEGMIAGIKAAAPGFRE |
| | WTENMLTKNYTWELFSNHGAWGAHANSLEMVHNTVHFLIGRDPT |
| | LDPLVPGHMGSVPHAAFDPIFWMHHCNVDRLLALWQTMNYDVY |
| | VSEGMNREATMGUPGQVLTEDSPLEPFYTKNQDPWQSDDLEDWE |
| | TLGFSYPDFDPVKGKSKEEKSVYINDWVHKHYG |

### EXAMPLES

### Example 1: Anti-HER2 Antibodies Conjugated to Payloads

This Example describes conjugation of two or more payloads to different regions of an antibody.

An exemplary antibody, such as Trastuzumab, with linkers are modified as follows: Heavy chains comprising a terminal tag ranging in length from -XY to -XXXXY, where X is any amino acid, are combined at a 1:100 ratio of tyrosinase enzyme to antibody in the presence of a cysteine nucleophile in 5× excess relative to the antibody. After 1 hr at 22° C., the heavy chains of the Trastuzumab antibodies are shown to be modified by both a Catenase and the tyrosinase from Agaricus Bisporus (abTYR). Light chains comprising a terminal tag ranging in length from -XXXY to -XXXXXY, where X is any amino acid but at least two X are negatively charged amino acids, are shown to react selectively with a Catenase, but not with abTYR, under similar conditions to the above.

Nucleophile-payload molecules are synthesized by combining NHS-Val-Cit-Payload with either 2(4-aminoethyl)-aniline or cysteamine for 24 hrs, and are then quenched by 50/50 dilution with water.

The light chain of antibodies are modified to contain negatively charged C-terminal tags (eg -EEEY). The heavy chain of the antibodies are modified to contain short, neutral tags (-SGGY or -SGY). The antibodies are expressed and purified according to routine procedures. The antibodies are then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM. Two payloads comprising a thiol are synthesized as described above, and are reduced prior to using in the tyrosinase reaction by running the payloads through an immobilized TCEP resin.

Payload A is attached to the heavy chain -SGGY tag by combining the antibodies with mushroom tyrosinase at 3% molar ratio of the enzyme and a 5× excess of Payload A relative to the antibody for 1 hour at RT with continuous shaking. After completion, conjugated antibodies are purified by either hydrophobic interaction chromatography (HIC) or size exclusion chromatography (SEC) according to established protocols. A Protein A column may also be used for this step.

To attach the second payload, antibodies modified with Payload A are combined with Payload B, which contains a thiol, at a 5× molar ratio, prepared as above, and 1-2% molar ratio of a Catenase (a modified tyrosinase from B. megaterium). Reactions are run for 1 hour at RT with shaking and are then purified via protein A column according to established procedures.

An antibody is thus generated with two different payloads conjugated to different portions of the antibody.

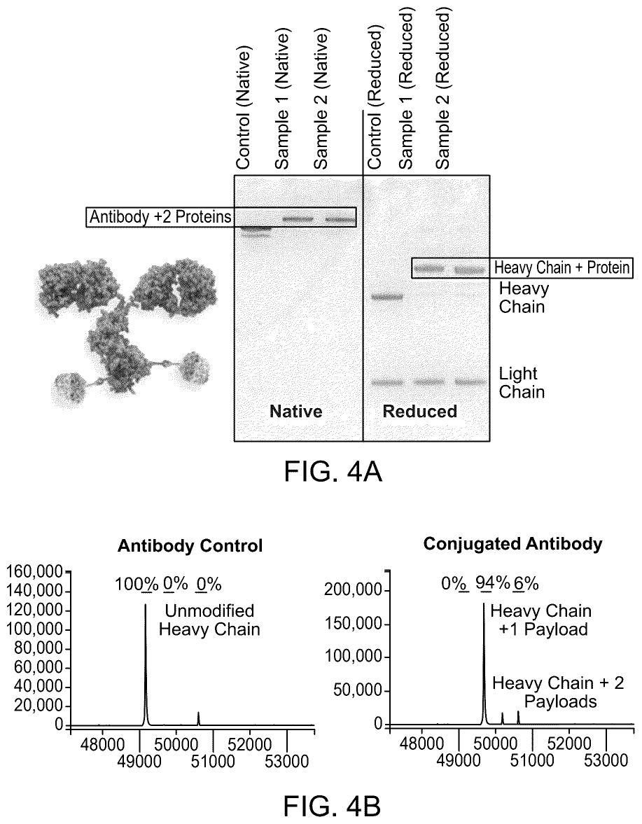

The modifications to the heavy chain are quantified by mass spectrometry. At least 90% of the exemplary heavy chain is modified to have at least 1 payload conjugated to it by the Catenase reaction (FIG. 4B, Panel B).

### Example 2: Exemplary Antibodies Conjugated to Multiple Payloads

This Example describes conjugation of at least two payloads to different regions of an antibody.

An exemplary antibody with linkers is modified as follows: Heavy chains comprising a terminal tag ranging in length from -XY to -XXXXY, where X is any amino acid, are combined at a 1:100 ratio of tyrosinase enzyme to antibody in the presence of a cysteine nucleophile in 5× excess relative to the antibody. After 1 hr at 22° C., the heavy chains of the Trastuzumab antibodies are shown to be modified by both a Catenase and the tyrosinase from Agaricus Bisporus (abTYR). Light chains comprising a terminal tag ranging in length from -XXXY to -XXXXXY, where X is any amino acid but at least two X are negatively charged amino acids, are shown to react selectively with a Catenase, but not with abTYR, under similar conditions to the above.

Nucleophile-payload molecules are synthesized by combining NHS-Val-Cit-Payload with either 2(4-aminoethyl)-aniline or cysteamine for 24 hrs, and are then quenched by 50/50 dilution with water.

The light chain of antibodies are modified to contain negatively charged C-terminal tags (eg -EEEY). The heavy chain of the antibodies are modified to contain short, neutral tags (-SGGY or -SGY). The antibodies are expressed and purified according to routine procedures. The antibodies are then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM. Two payloads comprising a thiol are synthesized as described above, and are reduced prior to using in the tyrosinase reaction by running the payloads through an immobilized TCEP resin.

Payload A is attached to the heavy chain -SGGY tag by combining the antibodies with mushroom tyrosinase at 3% molar ratio of the enzyme and a 5× excess of Payload A relative to the antibody for 1 hour at RT with continuous shaking. Payload A can also incorporate at natural tyrosine residues. After completion, conjugated antibodies are purified by either hydrophobic interaction chromatography (HIC) or size exclusion chromatography (SEC) according to established protocols. A Protein A column may also be used for this step.

To attach the second payload, antibodies modified with Payload A are combined with Payload B, which contains a thiol, at a 5× molar ratio, prepared as above, and 1-2% molar ratio of a Catenase (a modified tyrosinase from B. megaterium). Reactions are run for 1 hour at RT with shaking and are then purified via protein A column according to established procedures.

An antibody is thus generated with at least two different payloads conjugated to different portions of the antibody.

The modifications to the heavy chain are quantified by mass spectrometry. At least 90% of the exemplary heavy chain is modified to have at least 1 payload conjugated to it by the Catenase reaction, while about 30% of the heavy chain polypeptides have been modified to have at least two of Payload A conjugated to it (Panel C of FIG. 5). At least 70% of the exemplary light chain is modified to have at least 1 payload conjugated to it by the Catenase reaction, while about 2% of the light chain polypeptides have been modified to have at least two of Payload B conjugated to it (Panel D of FIG. 5).

### Example 3: Exemplary Antibodies Conjugated to Multiple Payloads Through Temperature Control

This Example describes the effect of performing the steps of antibody conjugation with a Catenase using a similar protocol described in Example 1 but with a change to the temperature of the reactions performed.

The heavy chains and light chains of an exemplary antibody were prepared similar to what was described in Example 1. Payload A was attached to the heavy chain -SGGY tag by combining the antibodies with mushroom tyrosinase at 3% molar ratio of the enzyme and a 5× excess of Payload A relative to the antibody for 1 hour at 4° C. with continuous shaking. After completion, conjugated antibodies were purified by either hydrophobic interaction chromatography (HIC) or size exclusion chromatography (SEC) according to established protocols. A Protein A column may also be used for this step.

To attach the second payload, antibodies modified with Payload A were combined with Payload B, which contains a thiol, at a 5× molar ratio, prepared as above, and 1-2% molar ratio of a Catenase (a modified tyrosinase from B. megaterium). Reactions were run for 1 hour at 37° C. with shaking and were then purified via protein A column according to established procedures.

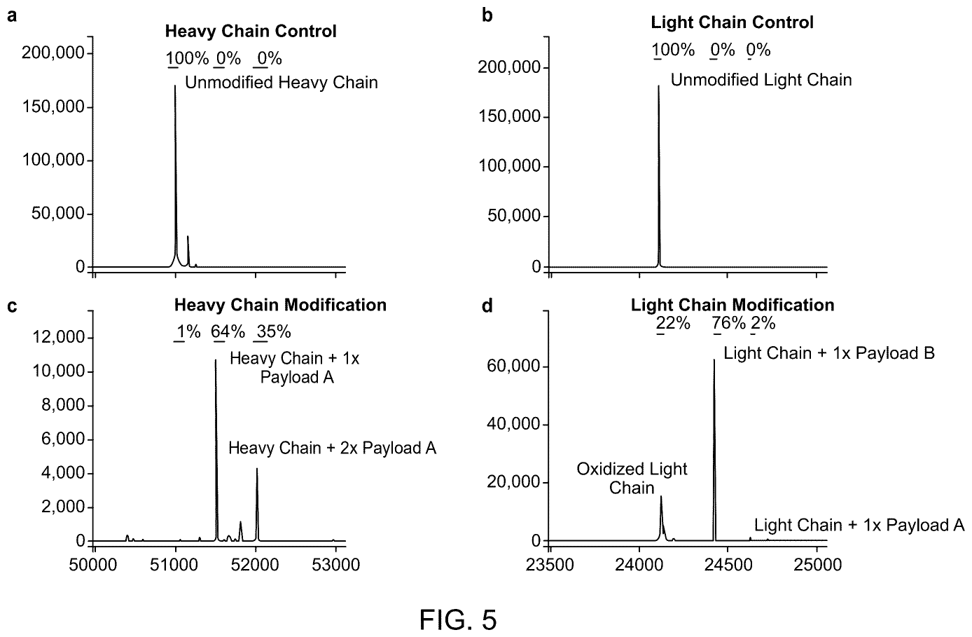

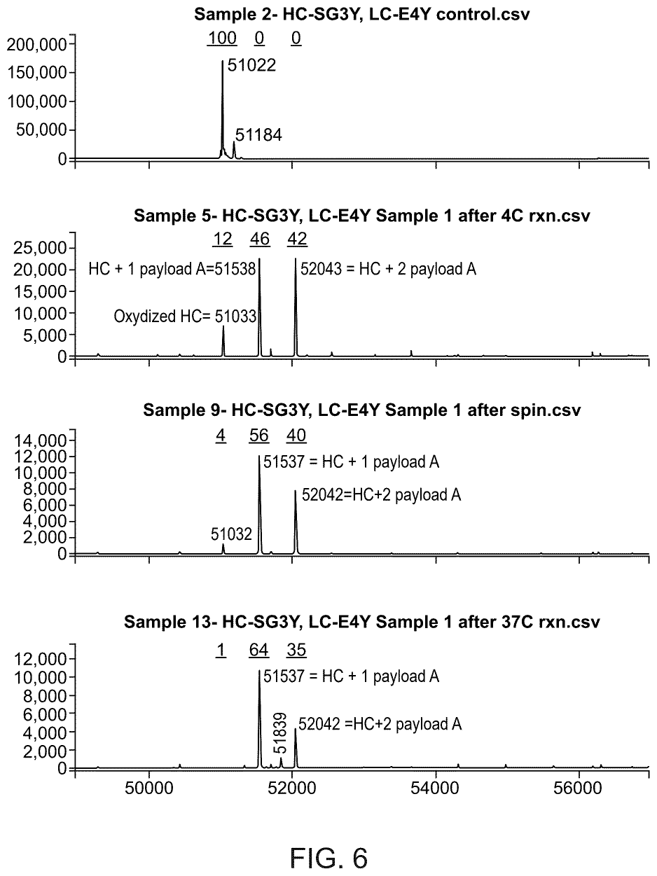

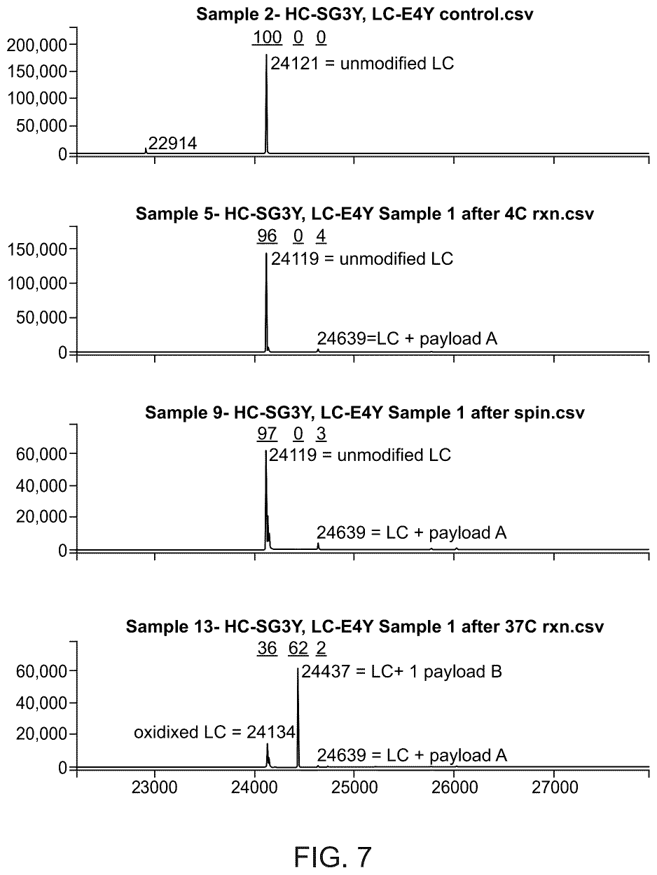

An antibody was thus generated with two different payloads conjugated to different portions of the antibody. FIG. 6 and FIG. 7 show exemplary mass spectra of the heavy chain and light chains, respectively, before, after the reaction for Payload A, after cleanup, and after the reaction for Payload B. The spectra show that the heavy chain was not modified with Payload B after the reaction to conjugate Payload B to the light chain.

### Example 4: Exemplary Antibodies Conjugated to Multiple Payloads Through Deglycosylation

This Example describes the effect of deglycoslyation of an antibody that was then conjugated to an exemplary payload with a Catenase enzyme.

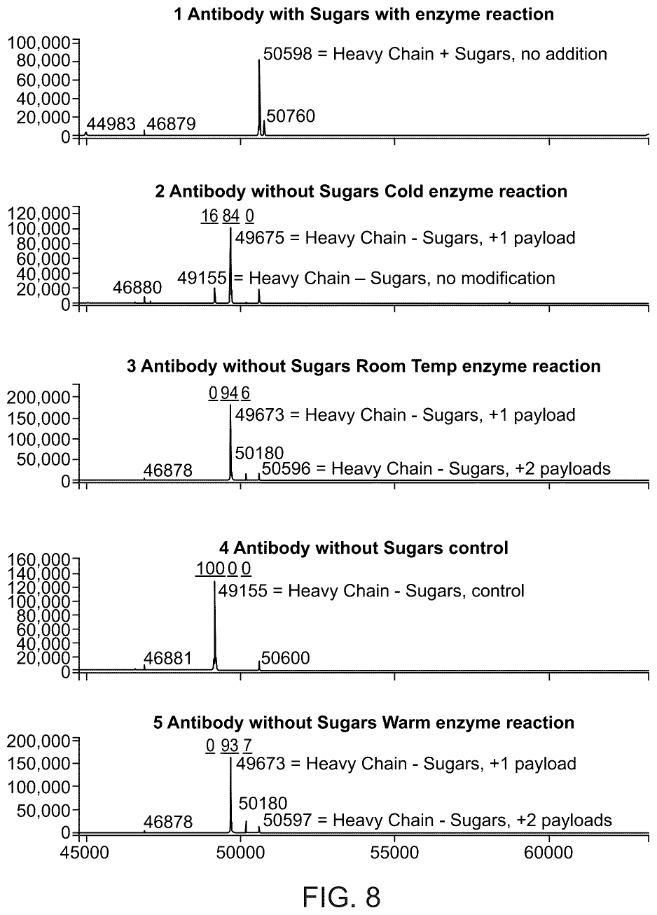

An exemplary antibody was deglycosylated through routine procedures known in the art. This exemplary antibody contains endogenous tyrosines. The deglycosylated exemplary antibody was then incubated with exemplary Payload A and a Catenase enzyme at 4° C. The resulting mass spectra analyzing this antibody is shown in FIG. 8, second graph, showing Payload A was successfully conjugated to the antibody. The antibody with natural sugars (glycosylated) was also incubated with Payload A and an enzyme, but no conjugation product was observed (FIG. 8, first mass spectra). The deglycosylated exemplary antibody was then incubated with exemplary Payload A and a Catenase enzyme at room temperature. The resulting mass spectra analyzing this antibody is shown in FIG. 8, third graph, showing Payload A was successfully conjugated to the antibody. The deglycosylated exemplary antibody was then incubated with exemplary Payload A and a Catenase enzyme at 37° C. The resulting mass spectra analyzing this antibody is shown in FIG. 8, fifth graph, showing Payload A was successfully conjugated to the antibody.

### Example 5: Exemplary HER2 Antibody Conjugated to Multiple Payloads

This Example describes conjugation of at least two payloads to different regions of a HER2 antibody.

An exemplary HER2 antibody with linkers was modified as follows: Heavy chains comprising a terminal tag ranging in length from -XY to -XXXXY, where X is any amino acid, were combined at a 1:100 ratio of tyrosinase enzyme to antibody in the presence of a cysteine nucleophile in 10×-15× excess relative to the antibody. After 1 hr, the heavy chains of the HER2 antibodies were shown to be modified by both a Catenase and the tyrosinase from Agaricus Bisporus (abTYR). Light chains comprising a terminal tag ranging in length from -XXY to -XXXXXY, where X is any amino acid but at least two X were negatively charged amino acids, were shown to react selectively with a Catenase, but not with abTYR, under similar conditions to the above.

Nucleophile-payload molecules were synthesized by combining NHS-Val-Cit-Payload with either 2(4-aminoethyl)-aniline or cysteamine for 24 hrs, and were then quenched by 50/50 dilution with water.

The light chain of antibodies were modified to contain negatively charged C-terminal tags (eg -EEEY). The heavy chain of the exemplary HER2 antibodies were modified to contain short, neutral tags (-SGGY or -SGY). The exemplary HER2 antibodies were expressed and purified according to routine procedures. The exemplary HER2 antibodies were then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM. Two payloads comprising a thiol were synthesized as described above, and were reduced prior to using in the tyrosinase reaction by running the payloads through an immobilized TCEP resin.

Payload A, a small molecule topoisomerase inhibitor, was attached to the heavy chain -SGGY tag by combining the antibodies with mushroom tyrosinase at 3% molar ratio of the enzyme and a 5×-15× (e.g., 10× or 15×) excess of Payload A relative to the antibody for 1 hour at RT with continuous shaking. Payload A can also incorporate at natural tyrosine residues. After completion, conjugated antibodies were purified by either hydrophobic interaction chromatography (HIC) or size exclusion chromatography (SEC) according to established protocols. A Protein A column may also be used for this step.

To attach the second payload, antibodies modified with Payload A were combined with Payload B, a small molecule tubulin inhibitor, which contains a thiol, at a 5×-15× molar ratio, prepared as above, and 1-2% molar ratio of a Catenase (a modified tyrosinase from B. megaterium). Reactions were run for 1 hour at RT without shaking and were then purified via SEC, HIC, or buffer exchanged.

An exemplary HER2 antibody was thus generated with at least two different payloads conjugated to different portions of the antibody that had different tags.

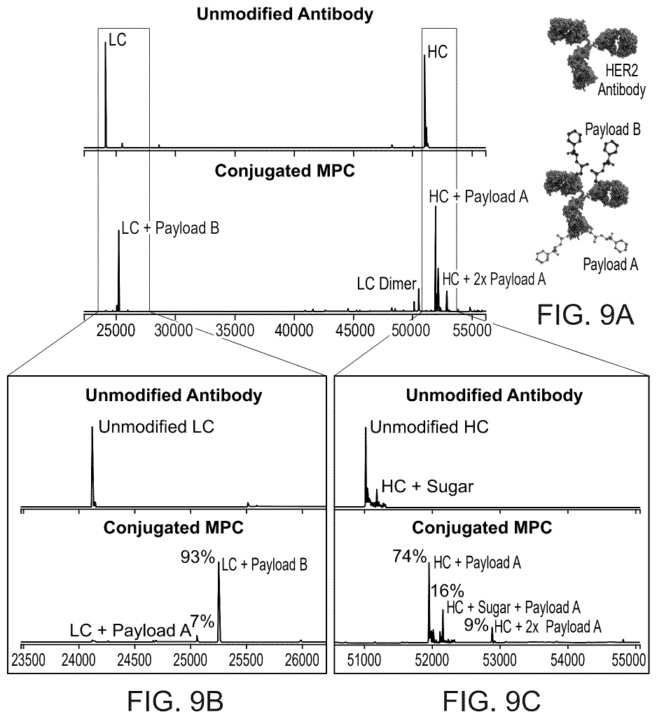

The modifications to the heavy chain were quantified by mass spectrometry. At least 90% of the exemplary heavy chain is modified to have at least 1 payload conjugated to it by the Catenase reaction, while about 9% of the heavy chain polypeptides have been modified to have at least two of Payload A conjugated to it (FIG. 9C). At least 93% of the exemplary light chain is modified to have at least 1 payload conjugated to it by the Catenase reaction (FIG. 9B).

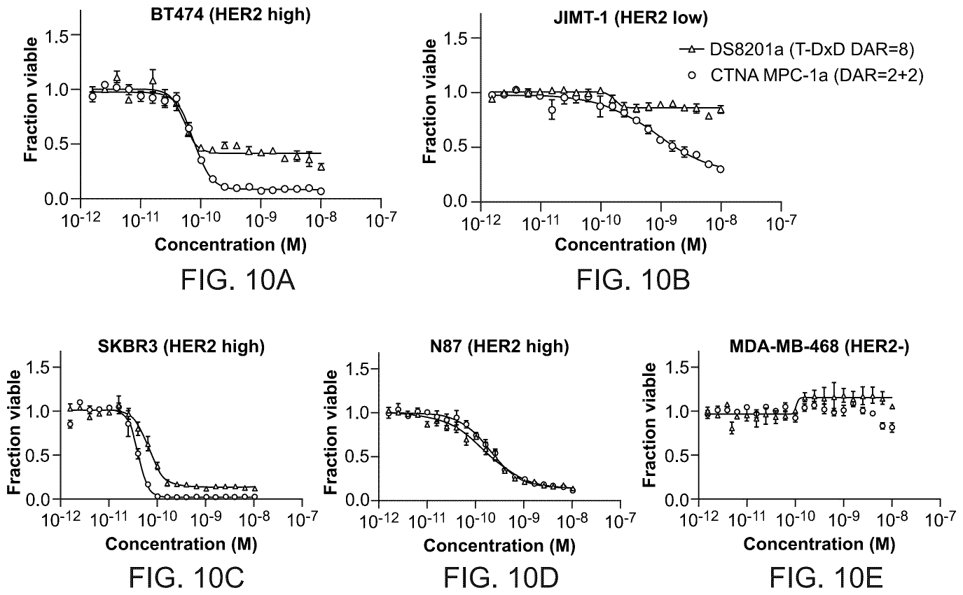

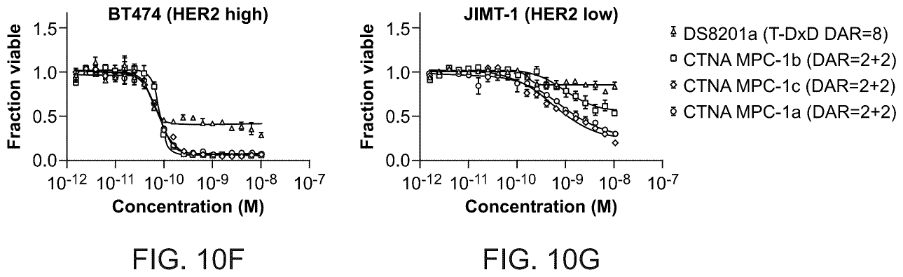

The exemplary HER2 antibody conjugates were tested in cell lines of interest. Cellular inhibition was measured via Alamar Blue Assay across 20 concentration points for each compound (n=4, FIG. 10A-10G). The exemplary HER2 antibody conjugates were tested in comparison to Ds8201a (T-Dxd), and the exemplary HER2 antibody conjugates showed significant increases in total inhibition. The exemplary HER2 antibody conjugates showed significant improvement in cell inhibition in high expressing HER2 cell lines BT474 and SKBR3 and low expressing HER2 cell lines JIMT-1, with equivalent activity in N87 cells. Both compounds show high selectivity through lack of inhibition in HER2 negative MDA-MB-468 cells.

The greater stability of the exemplary HER2 antibody conjugates and low, defined DAR of the reaction offers the incorporation of higher potency payloads with differentiated mechanism of actions to create more efficacious targeted therapies.

### Example 6: Tumor Growth in Mice Treated with an Exemplary HER2 Antibody Conjugated to Multiple Payloads

This Example describes a mouse study to compare the activity of an exemplary HER2 antibody conjugated to a topoisomerase inhibitor and a tubulin inhibitor in comparison to Ds8201a (T-Dxd).

SCID beige mice were injected with 5×10⁶ JIMT1 cells in 100 μL of PBS per mice in 100 μL Matrigel. JIMT1 is a human breast cancer xenograft model cell line. The tumors were allowed to grow in the mice until reaching a volume of 100 mm³. The mice were then randomly separated into 5 groups of 8-10 mice, which were then treated with control (PBS), Ds8201a (T-Dxd), which has a DAR of 8, or MPC (exemplary HER2 antibody conjugated to a topoisomerase inhibitor and a tubulin inhibitor), which has a DAR of 2+2. The treatment of the 5 groups is detailed below in Table A.

TABLE A

| | | N (# of | |
|---|---|---|---|
| Group | Treatment | mice) | Dose (Tail vein) |
| 1 | Vehicle control | 8 | 200 μL |
| | (PBS) | | |
| 2 | Ds8201a (T-Dxd) | 8 | 4 mg/kg (200 μL) |
| 3 | MPC dose 1 | 10 | 1 mg/kg (200 μL) |
| 4 | MPC dose 2 | 10 | 3 mg/kg (200 μL) |
| 5 | MPC dose3 | 10 | 8 mg/kg (200 μL) |

“MPC” represents the exemplary HER2 antibody conjugated to a topoisomerase inhibitor and a tubulin inhibitor.

Tumor volume (length×width×height) was measured twice per week with calipers, such as electronic calipers, and blood (plasma) samples were taken once per week. Body weight was measured twice per week.

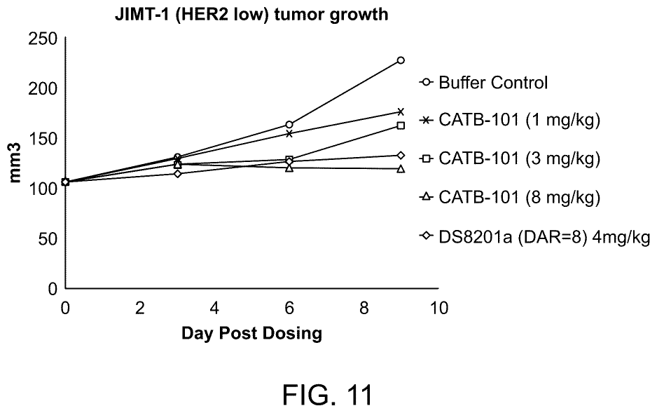

The average tumor growth (mm³) over time (days post dosing) is graphed in FIG. 11. The exemplary HER2 antibody conjugated to a topoisomerase inhibitor and a tubulin inhibitor shows equal or better activity in slowing down the growth of the tumor volume as compared to Ds8201a (T-Dxd) in comparison to the control. Notably, the exemplary HER2 antibody conjugated to a topoisomerase inhibitor and a tubulin inhibitor has a DAR of 2+2, which is half of the DAR of Ds8201a (T-Dxd), which has a DAR of 8.

### Example 7: Exemplary Modified Antibody Conjugated to Payload with Temperature Control

This Example describes temperature-selective conjugation of a payload to a heavy chain of an exemplary antibody.

In an exemplary antibody, such as Trastuzumab, the heavy chain was modified to comprise a terminal tag of SGGGY. The modified antibody was expressed and purified according to routine procedures. The modified antibody was then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM.

The modified trastuzumab was combined at a 1:100 ratio of Catenase (SEQ ID NO: 2) to antibody in the presence of an exemplary cysteine containing drug payload, Exatecan, in 10× excess relative to the antibody. The reaction was run for 1 hour at 4° C. with shaking. The modified antibody was then purified via ultrafiltration and/or diafiltration.

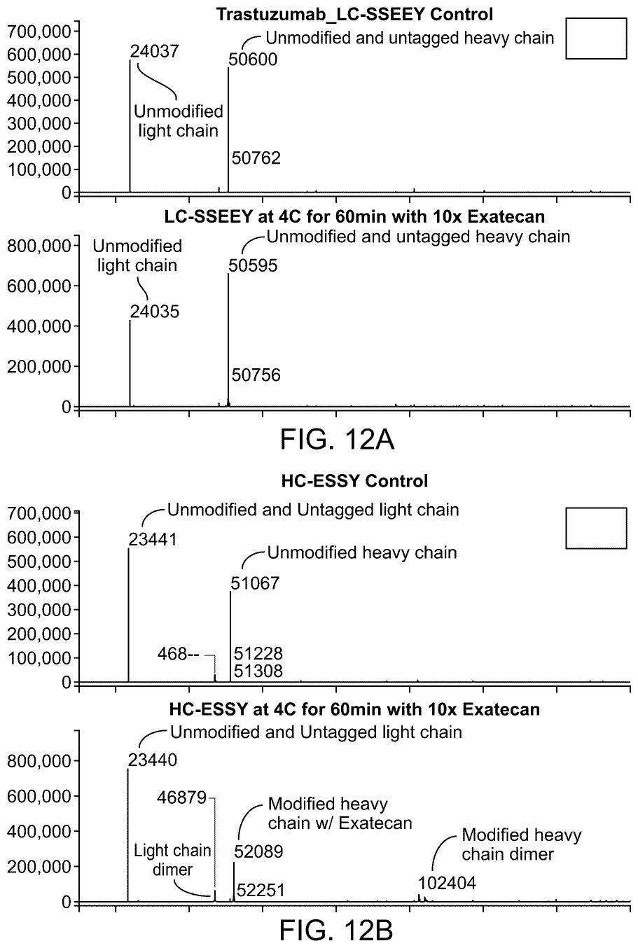

After the reaction at 1 hr and 4° C., Exatecan was conjugated to the heavy chain of the modified Trastuzumab by Catenase, as can be seen by the mass spectrometry data in FIG. 12B. Exatecan was not conjugated to the light chains of the modified Trastuzumab, as can be seen by the mass spectrometry data in FIG. 12B. A mass difference of −5 Da was observed in the heavy chain from the control (top graph) to the reaction (bottom graph) due to a change in buffer pH.

In another exemplary antibody, such as Trastuzumab, the light chain was modified to comprise a terminal tag of SSEEEY. After a reaction with Catenase at 1 hr and 4° C., Exatecan was not conjugated to the light chains of the modified Trastuzumab (FIG. 12A).

Therefore, the Catenase selectively conjugated Exatecan to the heavy chains with a terminal tag ESSY of the modified antibody when the reaction conditions used were 4° C. for 1 hr.

### Example 8: Exemplary Modified Antibody Conjugated to Payload with Temperature Control

This Example describes temperature-selective conjugation of a payload to the light chain and heavy chain of an exemplary antibody.

An exemplary antibody, such as Trastuzumab, in which the heavy chain was modified to have the terminal tag SGGGY and the light chain was modified to have the terminal tag .EEEEY. The modified antibody was expressed and purified according to routine procedures. The modified antibody was then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM.

The modified trastuzumab was combined at a 1:100 ratio of Catenase (SEQ ID NO: 2) to antibody in the presence of an exemplary cysteine containing drug payload, Exatecan, in 12× excess relative to the antibody. The reaction was run for 90 min at 30° C. with shaking. The modified antibody was then purified via protein A column according to established procedures.

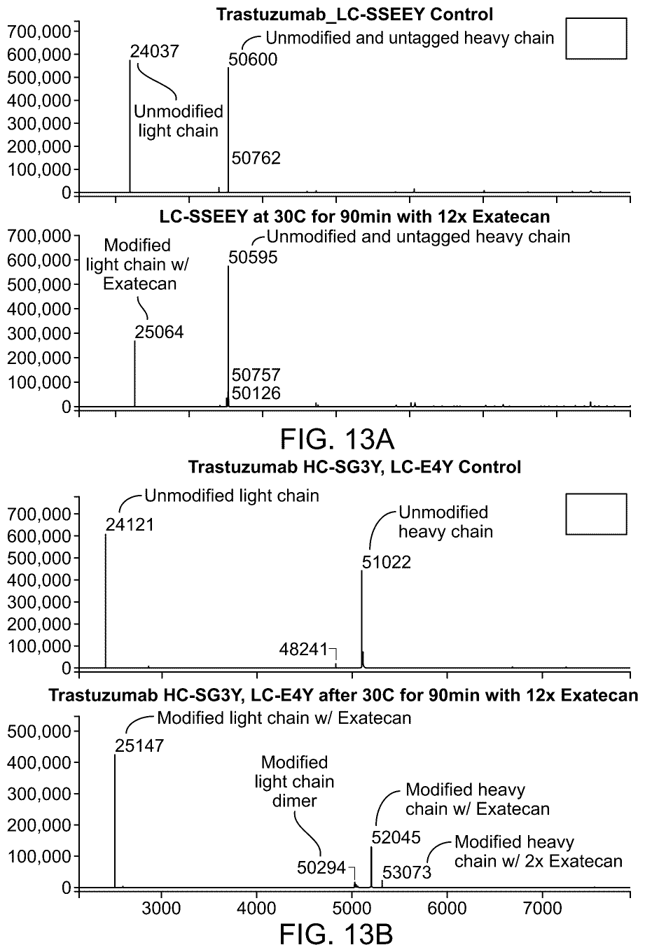

After the reaction at 90 min at 30° C., Exatecan was conjugated to the heavy chain and light chain of the modified Trastuzumab by Catenase (SEQ ID NO: 2), as can be seen by the mass spectrometry data in FIG. 13B.

Another modified Trastuzumab was prepared in which the light chain was modified to have a terminal tag of SSEEY. After the reaction with Catenase at 90 min at 30° C., the exemplary payload, Exatecan, was conjugated to the light chains of the modified antibody (FIG. 13A).

Therefore, the Catenase conjugated Exatecan to the light chain and heavy chain of the modified antibody comprising a terminal tag when the reaction conditions used were 90 min at 30° C.

### Example 9: Exemplary Modified Antibody Conjugated to Payload with Temperature Control

This Example describes temperature-selective conjugation of a payload to the light chain and heavy chain of an exemplary antibody, including a non-terminal conjugation site in the heavy chain.

An exemplary antibody, such as Trastuzumab, was modified as follows. The heavy chain was modified to comprise the terminal tag ESSY and to include the mutations S298G and T299 Å to aglycosylate the antibody and expose a non-terminal tyrosine on a loop for modification, and the light chain was modified to comprise the terminal tag SSEEY. The modified antibody was expressed and purified according to routine procedures. The modified antibody was then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM.

The modified trastuzumab was combined at a 1:100 ratio of Catenase (SEQ ID NO: 2) to antibody in the presence of an exemplary cysteine containing drug payload, Exatecan, in 10× excess relative to the antibody. The reaction was run for 60 min at 4° C. with shaking. The modified antibody was then purified via protein A column according to established procedures.

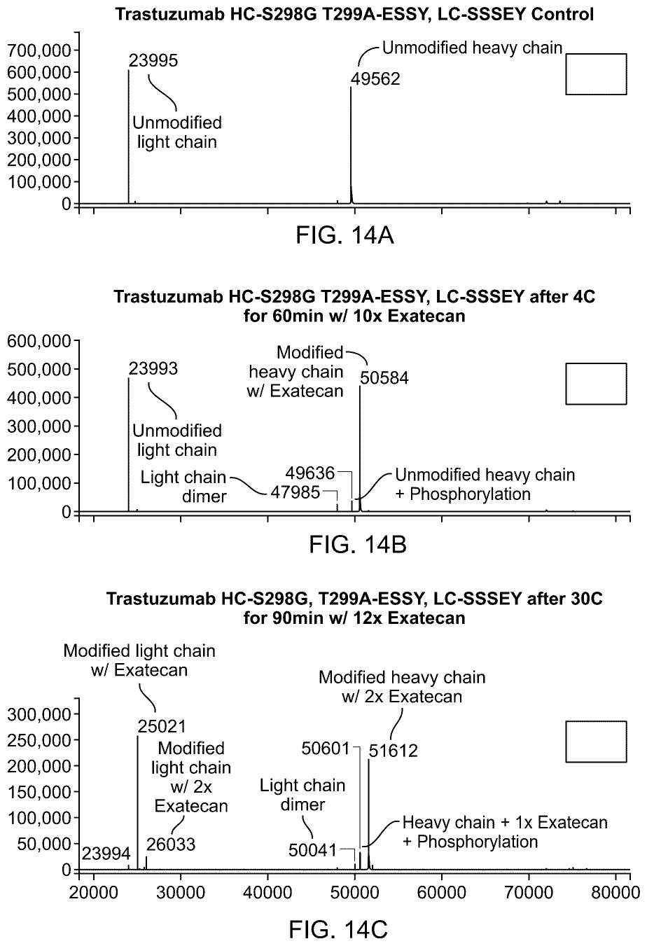

After the reaction at 60 min at 4° C., Exatecan was conjugated only to the heavy chain of the modified Trastuzumab by Catenase (SEQ ID NO: 2), as can be seen by the mass spectrometry data in FIG. 14A and FIG. 14B. Notably, based on the mass difference between the mass spec data in FIG. 14A and FIG. 14B, only one Exatecan had been conjugated to the heavy chain, meaning only one tyrosine had been reacted by Catenase, likely the more accessible tyrosine on the terminal tag of the heavy chain. Exatecan was not conjugated to the light chain of the modified Trastuzumab.

The modified Trastuzumab of this first reaction was then combined at a 1:100 ratio of Catenase (SEQ ID NO: 2) to the modified Trastuzumab in the presence of Exatecan in 12× excess. The reaction was run for 90 min at 30° C. After the reaction for 90 min at 30° C., Exatecan was now also conjugated to light chain of the modified Trastuzumab by Catenase, as can be seen by the mass spectrometry data in FIG. 14C. Further, the mass spec data showed that now two Exatecan molecules were conjugated to the heavy chain.

Therefore, the Catenase (SEQ ID NO: 2) conjugated Exatecan selectively to the more accessible tyrosine on the terminal tag of the heavy chain at lower temperatures, while leaving the other tyrosine alone. Catenase then conjugated Exatecan to the light chain and to the less accessible non-terminal tyrosine in the loop of the heavy chain at the higher temperature of 30° C. in the second reaction. Notably, the same enzyme was used for both reactions.

### Example 10: Exemplary Modified Antibody Conjugated to More than One Payload with Temperature Control

This Example describes temperature-selective conjugation of more than one payload to the light chain and heavy chain of an exemplary antibody, including a non-terminal conjugation site in the heavy chain.

An exemplary antibody, such as Trastuzumab, was modified as follows. The heavy chain was modified to comprise the terminal tag ESSY and to include the mutations S298G T299 Å to aglycosylate the antibody and expose a non-terminal tyrosine on a loop for modification, and the light chain was modified to comprise the terminal tag SSEEY. The modified antibody was expressed and purified according to routine procedures. The modified antibody was then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM.

The protocol for conjugating at least two different payloads to a modified antibody that was followed is provided below.

### Protocol for DAR2+4 MPC Production

1. Prepare everything besides payload.
   1. Labeled reactions tubes with buffer and antibody over ice.
   2. 1:10 diluted Catenase (SEQ ID NO: 2).
   3. Antibodies prepared.
   4. 1:1 inhibitor mix (tropolone and L-proline).
   5. Tris(2-carboxyethyl)phosphine (TCEP)
2. Move reactions and tubes to fumehood.
3. Add 10× Payload #1 (MMAE) to each reaction tube for the ˜4° C. reaction.
4. Be sure tubes are already in the incubator to cool to ˜4° C.
5. Add Catenase (SEQ ID NO: 2) (1× equivalence) to the reaction and run for 45 minutes with 200 rpm shaking.
6. While reaction is running, prepare 100 kDa, 4 mL Millipore spin filters (regenerated cellulose membrane) by rinsing 3 times with water, followed by passing some reaction buffer through the membrane.
7. After 45 minutes quench with inhibitor mix.
8. Take a 40 μL sample.
9. Buffer exchange the reaction mixture using 4 mL 100 kDa spin filters (one per approximately 6 mL of reaction mixture) to remove excess Catenase, payload #1, and inhibitor mix.
10. Spin at 4,000 rcf.
11. Pool the flowthrough of each filter after each spin and measure the absorbance at A280 to track tropolone removal from the reaction mixture.
12. Ensure that the flowthrough absorbance reads 0 before proceeding to reaction 2.
13. At final spin, bring each reaction tube volume down to below volume of reaction 2 without inhibitor mix, enzyme, and MMAE added.
14. Measure exact volume and adjust with buffer accordingly
15. Remove 40 μL of reaction for inline measurements (e.g., yield, gel, mass spectrometry).
16. Add proper amounts of buffer, payload #2 (15× Exatecan), and Catenase (SEQ ID NO: 2) (1× equivalence) for proper final reaction volume and drug equivalence and run the reaction for 120 min at 30° C. with 200 rpm shaking.
17. Take a 40 μL inline sample at 90 min.
18. Wipe down payload vials and transfer back to −80 C.
19. After 120 min, quench the reaction with the proper amount of inhibitor.
20. Take 50 μL aliquot of reaction for inline measurements (e.g., gel, mas spec, endotoxin).
21. Freeze the sample for future use or store in fridge to buffer exchange the following day.

### Protocol for DAR2+2 MPC Production

1. Prepare everything besides payload outside of the fume hood:
   1. Labeled reactions tubes with buffer and antibody over ice.
   2. 1:10 diluted Catenase (SEQ ID NO: 2).
   3. Antibodies.
   4. 1:1 inhibitor mix (tropolone and L-proline).
   5. TCEP.
2. Move reactions and tubes to fumehood.
3. Add 7× Payload #1 (Exatecan) to each reaction tube for the 4° C. reaction.
   1. Done with tubes in the incubator to cool to 4° C.
4. Add Catenase (SEQ ID NO: 2) (1× equivalence) to the reaction and run for 15 minutes with no shaking.
   1. While reaction is running, prepare 100 kDa, 4 mL spin filters (regenerated cellulose membrane) by rinsing 3× with water, followed by passing some reaction buffer through the membrane.
5. After 45 minutes quench with inhibitor mix.
6. Take a 40 μL inline sample.
7. Buffer exchange the reaction mixture using 4 mL 100 kDa spin filters (one per approximately 6 mL of reaction mixture) to remove excess Catenase, payload #1, and inhibitor.
   1. Spin at 4000 rcf.
   2. Pool the flowthrough of each filter after each spin and measure the absorbance at A280 to track tropolone removal from the reaction mixture.
   3. Ensure that the flowthrough absorbance reads 0 before proceeding to reaction 2.
8. At final spin, bring each reaction tube volume down to below volume of reaction 2 without inhibitor mix, enzyme, and payload #2 (MMAE) added.
   1. Measure exact volume and adjust with buffer accordingly.
9. Remove 40 μL of reaction for inline measurements (e.g., yield, gel, MS).
10. Add proper amounts of buffer, payload #2 (10×MMAE), and Catenase (SEQ ID NO: 2) (1× equivalence) for proper final reaction volume and drug equivalence and run the reaction for 120 min at 30° C. with 200 rpm shaking.
   1. Take a 40 μL inline sample at 90 min.
   2. Prepare mass spec sample spreadsheet and vials during this reaction.
11. Wipe down payload vials and transfer back to −80° C.
12. After 120 min, quench the reaction with the proper amount of inhibitor.
13. Take 50 μL aliquot of reaction for inline measurements (e.g., gel, mass spec, endotoxin).
14. Freeze the sample for future use or store in fridge to buffer exchange the following day.

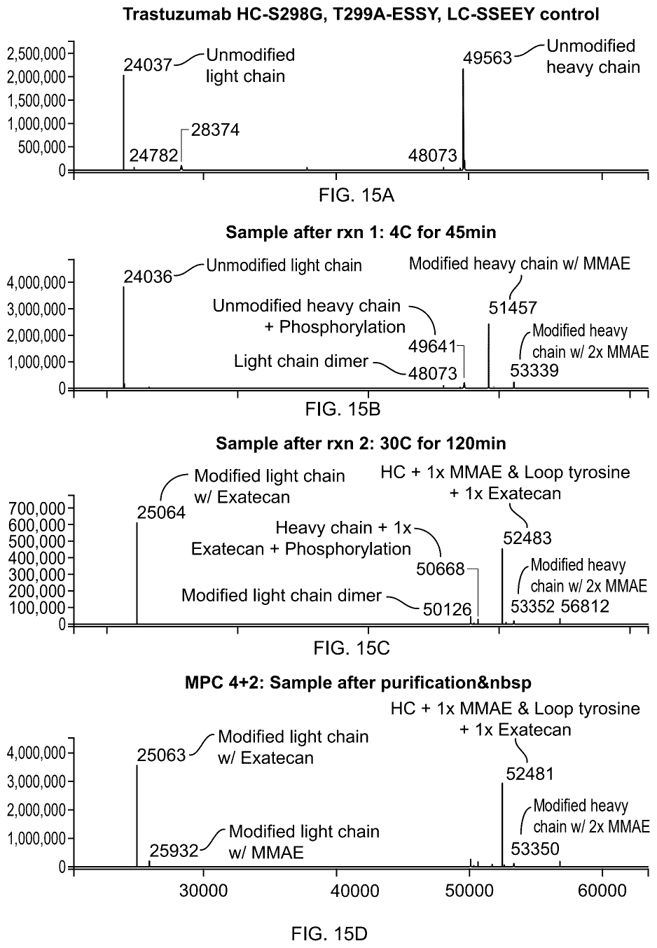

After the reaction for 45 min at 4° C., MMAE was conjugated only to the tyrosine on the terminal tag of the heavy chain by Catenase (SEQ ID NO: 2), as can be seen by the mass spectrometry data in FIG. 15A and FIG. 15B. Notably, based on the mass difference between the mass spec data in FIG. 15A and FIG. 15B, heavy chains has either one or two MMAE conjugated to it. MMAE was not conjugated to the light chain of the modified Trastuzumab.

The modified Trastuzumab of this first reaction was used for the second reaction with Exatecan. The reaction was run for 120 min at 30° C. After the reaction for 120 min at 30° C., Exatecan was conjugated to light chain of the modified antibody by Catenase (SEQ ID NO: 2), as can be seen by the mass spectrometry data in FIG. 15C. Further, the mass spec data show Exatecan was also conjugated to the loop tyrosine in the heavy chain. FIG. 15D shows the mass spectrometry of the modified antibody after purification.

Catenase conjugated a first payload, MMAE, selectively to the more accessible tyrosine on the terminal tag of the heavy chain at lower temperatures, and then the same Catenase was used to conjugate a second payload, Exatecan, to the light chain and the non-terminal tyrosine of the modified Trastuzumab. Therefore, multi-payload antibody drug conjugates can be produced using the same enzyme by simply adjusting the reaction temperature and time of reaction.

### Example 11: Exemplary Modified Antibody Conjugated to More than One Payload with a Variety of Terminal Tags

This Example describes temperature-selective conjugation of more than one payload to an exemplary modified antibody with a range of terminal tags comprising a tyrosine.

The methods described in Example 10 were generally used for the reactions described in this example. Modified antibodies were generated with different terminal tags on the heavy chain and light chain to demonstrate the range of exemplary terminal tags that allow for selective conjugation of the heavy chain and light chain under temperature controlled reactions.

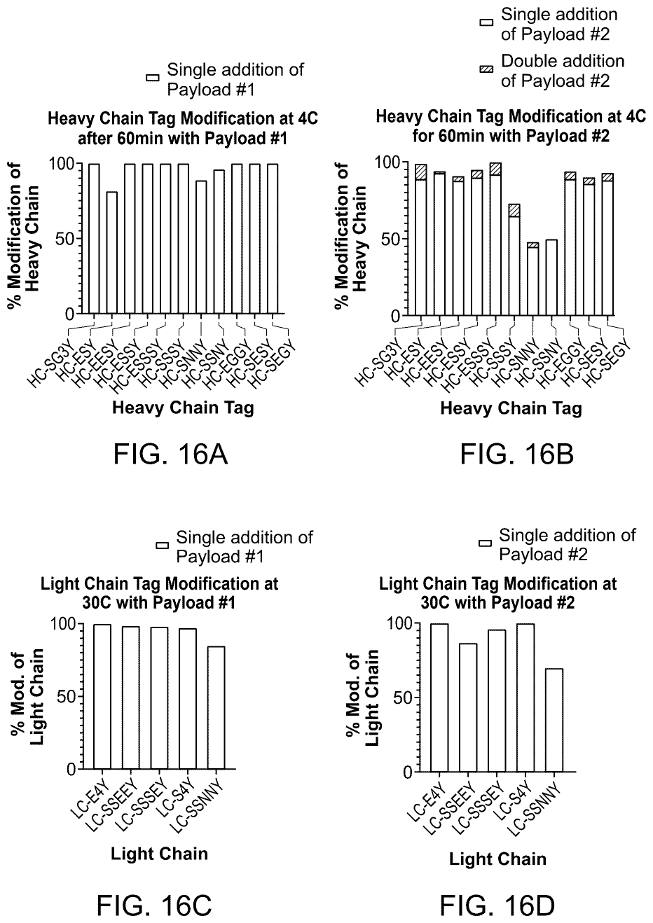

Exemplary antibodies were generated with a terminal tag on the heavy chain such as SGGGY, ESY, EESY, ESSY, ESSSY, SSSY, SNNY, SSNY, EGGY, SESY, and SEGY. Reactions at 4° C. for 60 min with any of these terminal tags and Catenase (SEQ ID NO: 2) showed that at least about 80% of the heavy chains were conjugated to exemplary payload #1 (FIGS. 16A-16D). Reactions at 4° C. for 60 min with any of these terminal tags showed that at least about 70% of the most of the heavy chains were conjugated to exemplary payload #2. Some heavy chains were modified with a second addition of payload #2 (FIG. 16B). A second payload can occasionally add into the same phenol ring as the first payload. This arises from re-oxidation of the thiol modified ring followed by subsequent addition of an additional thiol into the ring either adjacent or opposite the original modification on the ortho-quinone ring.

Exemplary antibodies were generated with a terminal tag on the light chain such as EEEEY, SSEEY, SSSEY, SSSSY, and SSNNY. Reactions at 30° C. with any of these terminal tags and Catenase (SEQ ID NO: 2) showed that at least about 85% of the lights chains were conjugated to exemplary payload #1 (FIG. 16C). Reactions at 30° C. with any of these terminal tags showed that at least about 70% of the lights chains were conjugated to exemplary payload #2 (FIG. 16D).

### Example 12: Exemplary Modified Antibody Conjugated to a Variety of Payloads

This Example describes conjugation of a variety of payloads to the light chain and heavy chain of an exemplary antibody.

An exemplary antibody, such as Trastuzumab, was modified to contain terminal tags as follows. The heavy chain was modified to comprise the terminal tag SGGGY and the light chain was modified to comprise the terminal tag EEEEY. The modified antibody was expressed and purified according to routine procedures. The modified antibody was then buffer exchanged into a Catenase reaction buffer. The Catenase reaction buffer can be 10-50 mM phosphate pH 6.5, 150 mM NaCl, and 2 mM EDTA or 10-100 mM Acetate pH 5.5, 0-10% sucrose 1 mM EDTA. The phosphate in the first listed buffer can be replaced with HEPES, Tris, Acetate, and MES buffers, from pH 5 to pH 9. Additionally, sucrose and glucose can be added to the buffer up to 15% v/v, Trehalose can be added at up to 300 mM, and EDTA can be added at up to 5 mM.

The modified trastuzumab was combined with Catenase (SEQ ID NO: 2) in the presence of an exemplary cysteine containing drug payload in 5× excess relative to the antibody. A variety of payloads were tested, including MMAE, doxorubicin, camptothecin, and exatecan. The reactions were run for 1 hour at 30° C. with shaking. The modified antibody was then purified via protein A column according to established procedures.

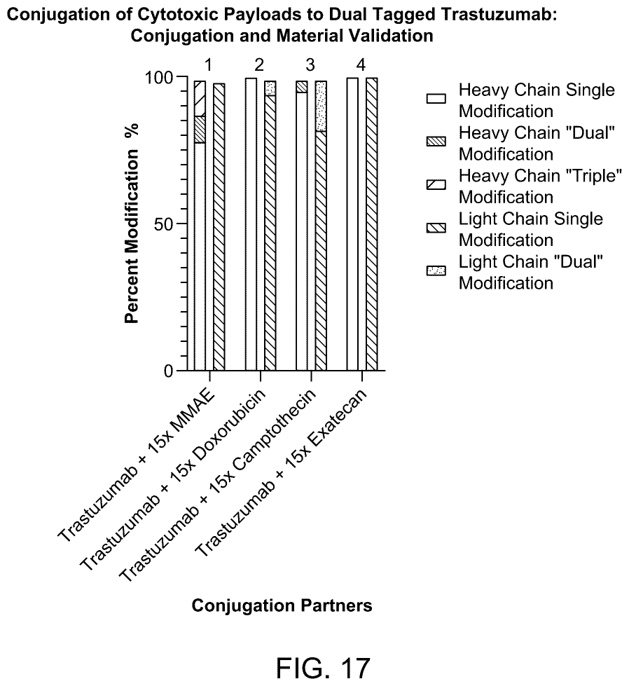

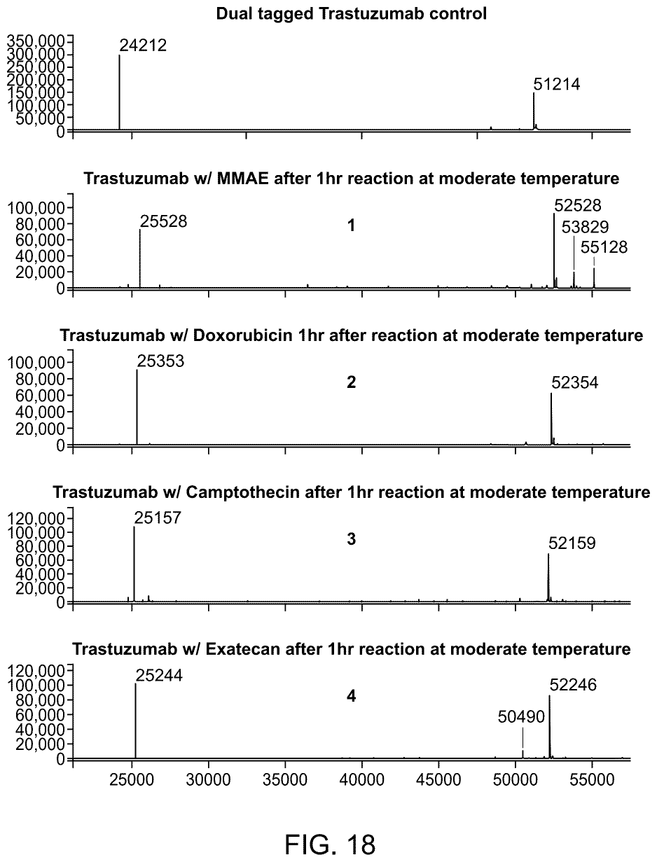

After the reaction at 30° C., all tested payloads were conjugated to the heavy chain and light chain of the modified Trastuzumab by Catenase (SEQ ID NO: 2), as can be seen by the graph and the mass spectrometry data in FIG. 17 and FIG. 18. While most heavy chains were conjugated to one payload molecule, some heavy chains were conjugated to two or three payload molecules. Some light chains were also conjugated to two payload molecules. As described herein, a second and/or third payload can occasionally add into the same phenol ring as the first payload. This arises from re-oxidation of the thiol modified ring followed by subsequent addition of an additional thiol into the ring either adjacent or opposite the original modification on the ortho-quinone ring. Full modification (more than 95% modification) of the heavy chain and light chain was observed for the reactions with all the exemplary payloads.

Therefore, Catenase conjugated a variety of exemplary payloads to an exemplary modified antibody.

### Example 13: Exemplary Modified Antibody Conjugated to a Variety of Payloads with Temperature Control

This Example describes conjugation of a variety of payloads to the heavy chain of an exemplary antibody under a temperature controlled reaction.

An exemplary antibody, such as Trastuzumab, was modified to contain terminal tags as follows. The heavy chain was modified to comprise the terminal tag SGGGY and the light chain was modified to comprise the terminal tag EEEEY. The modified antibody was expressed and purified according to routine procedures.

The modified trastuzumab was combined with Catenase (SEQ ID NO: 2) in the presence of an exemplary cysteine containing drug payload in 5× excess relative to the antibody. A variety of payloads were tested including MMAE, doxorubicin, camptothecin, and exatecan. The reactions were run for 1 hour at 4° C. with shaking. The modified antibody was then purified via protein A column according to established procedures.

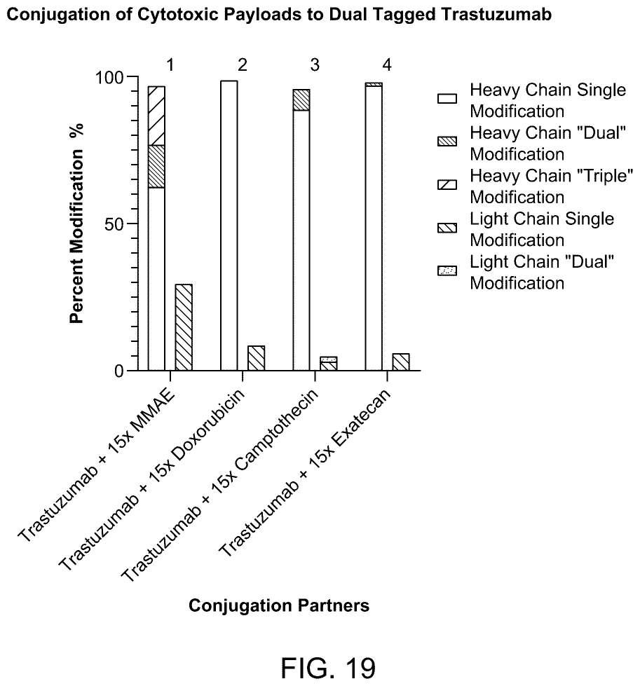

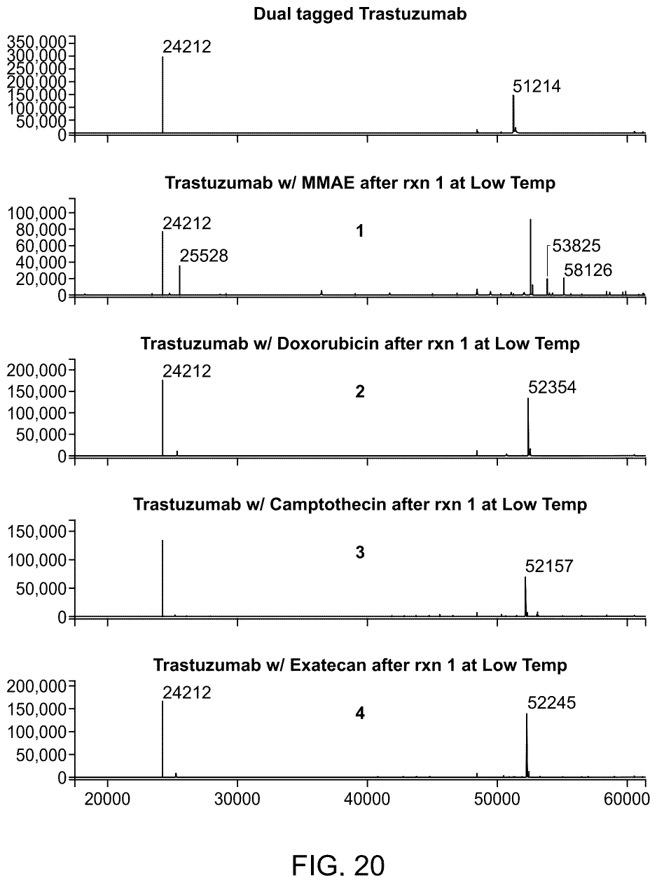

After the reaction at 4° C., the tested payload was conjugated to the heavy chain of the modified Trastuzumab by Catenase (SEQ ID NO: 2), as can be seen by the data in FIG. 19 and FIG. 20. While most heavy chains were conjugated to one payload molecule, some heavy chains were conjugated to two payload molecules or three payload molecules. Full modification (more than 95% modification) of the heavy chains was observed for the reactions with all the exemplary payloads. Minor amounts of the light chain of the modified antibody were conjugated to the exemplary payload, such as less than 30% of light chains in the reaction with MMAE and less than 10% of light chains in the reactions with the other exemplary payloads.

Therefore, Catenase selectively conjugated a variety of exemplary payloads to the heavy chain of an exemplary modified antibody under the reaction condition of low temperature (e.g., 4° C.).

The procedures disclosed herein in Examples 1-123 can be conducted with any polypeptide, such as any antibody, known in the art. It is understood by one skilled in the art of that the functionality present on various portions of the molecule should be compatible with the reagents and reactions proposed.

### Example 14: Synthesis of Exatecan with a Thiol-Containing Linker

This Example describes a synthesis of a payload, Exatecan, to a linker with a thiol. Exatecan is abbreviated as DXD in the scheme below.

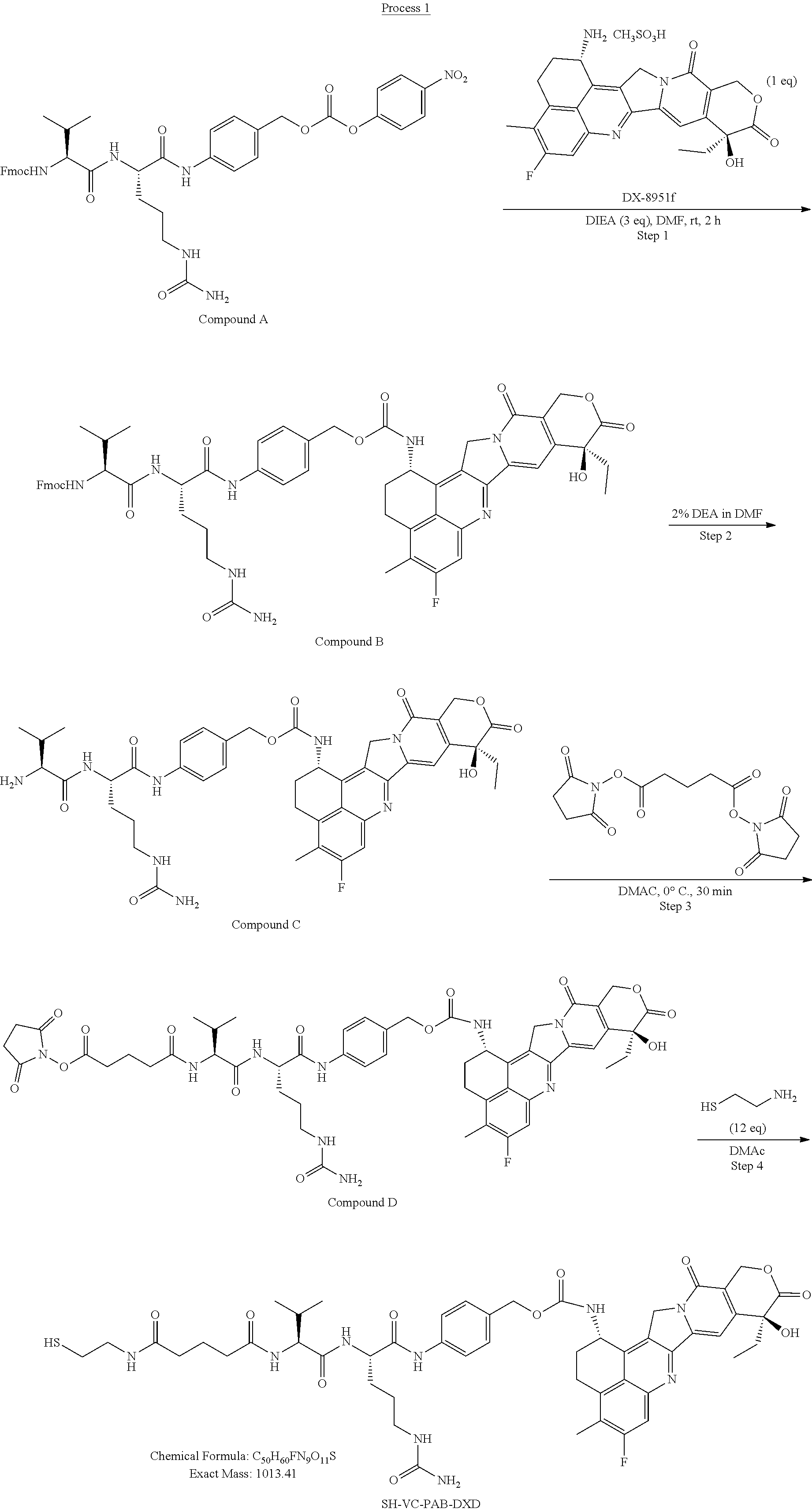

In Step 1, Compound A was reacted with DX-8951f in the presence of N,N-Diisopropylethylamine (DIEA) in dimethylformamide (DMF) at room temperature for two hours to provide Compound B. In the next step, Compound B was subjected to 2% Diethanolamine (DEA) in DMF to deprotect the Fmoc group and form Compound C. Then, Compound C was reacted with bis(2,5-dioxopyrrolidin-1-yl) glutarate in the presence of dimethylacetamide (DMAC) at 0° C., for 30 minutes to yield Compound D. Next, Compound D was reacted with 12 equivalents of 2-aminoethane-1-thiol in the presence of DMAC. The final SH-VC-PAB-DXD compound was purified, yielding 38 mg at 90% purity. [M+H]⁺ 1,014.35.

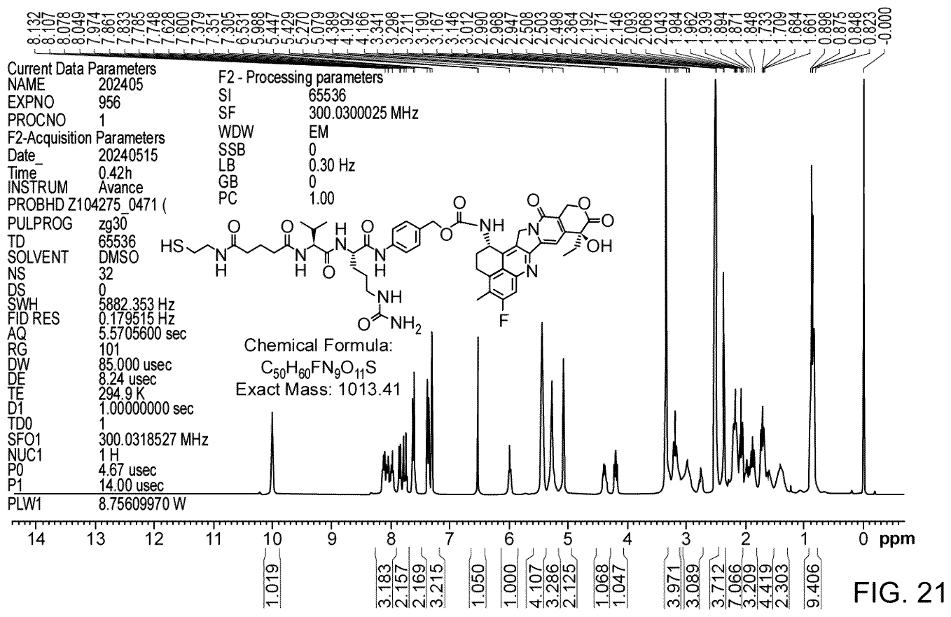

The NMR spectra of the final compound is provided in FIG. 21.

### Example 15: Synthesis of MMEA with a Thiol-Containing Linker

This Example describes a synthesis of a payload, MMEA, to a linker with a thiol.

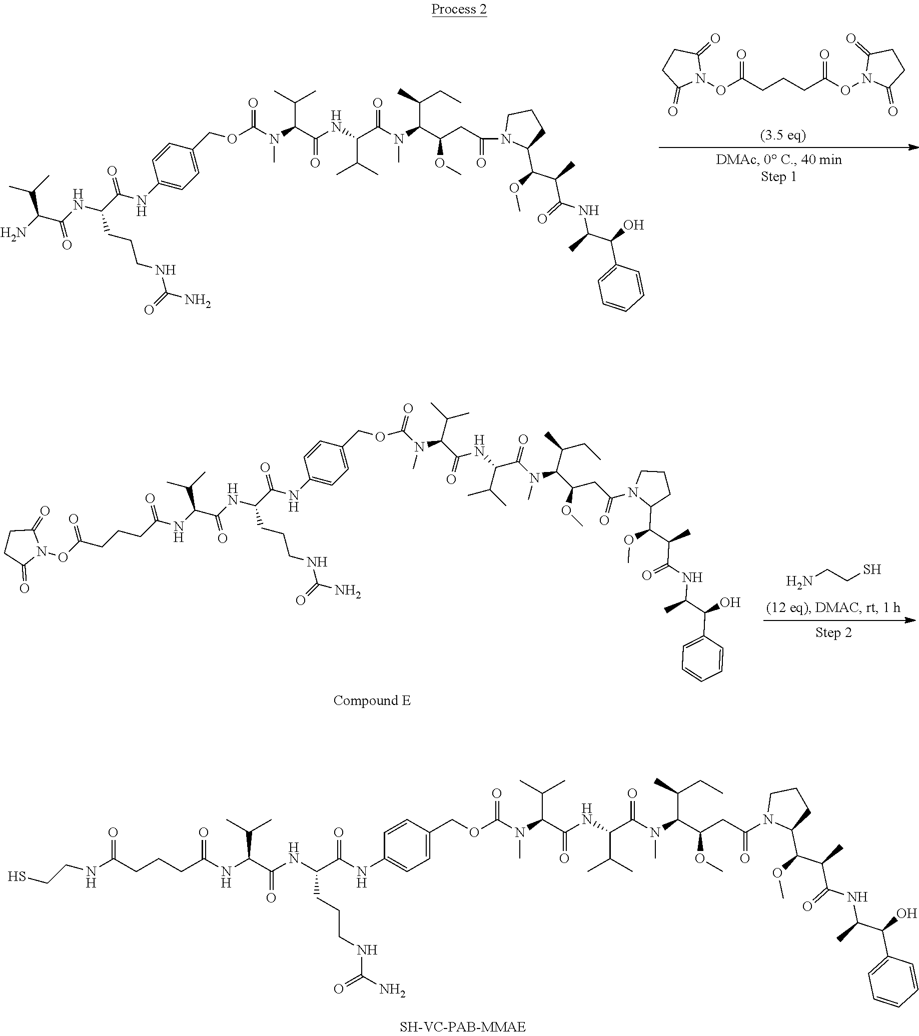

First, VC-PAB-MMAE was reacted with bis(2,5-dioxopyrrolidin-1-yl) glutarate in the presence of DMAC at 0° C. for 40 min. Next, Compound E was reacted with 12 equivalents of bis(2,5-dioxopyrrolidin-1-yl) glutarate in the presence of DMAC at room temperature for 1 hour. Finally, the SH-VC-PAB-MMAE compound was purified, yielding 59 mg. [M+H]: 649.20.

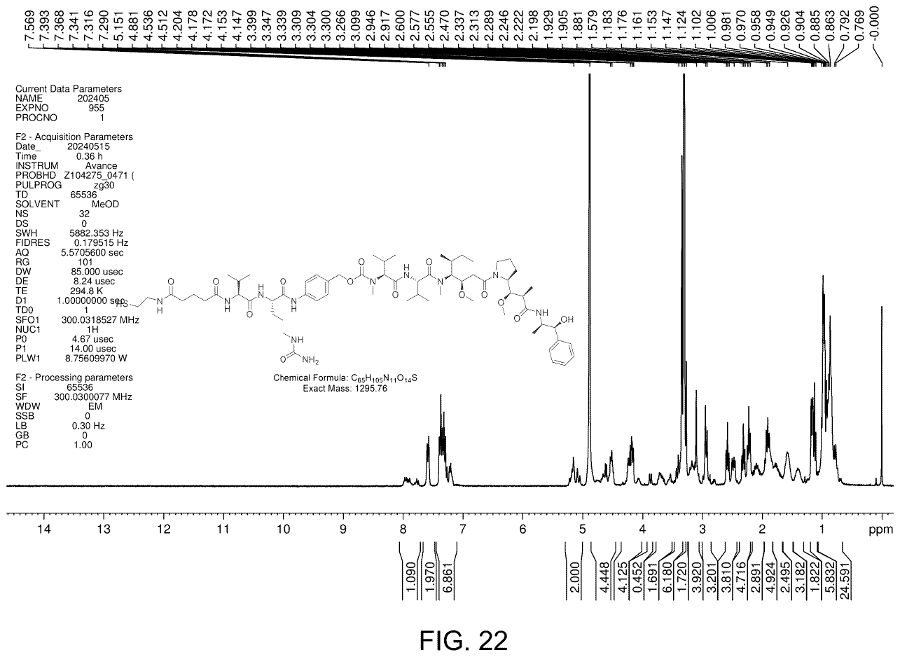

The NMR spectra of the final compound is provided in FIG. 22.

### Example 16: Synthesis of Camptothecin with a Thiol-Containing Linker

This Example describes a synthesis of a payload, Camptothecin, to a linker with a thiol.

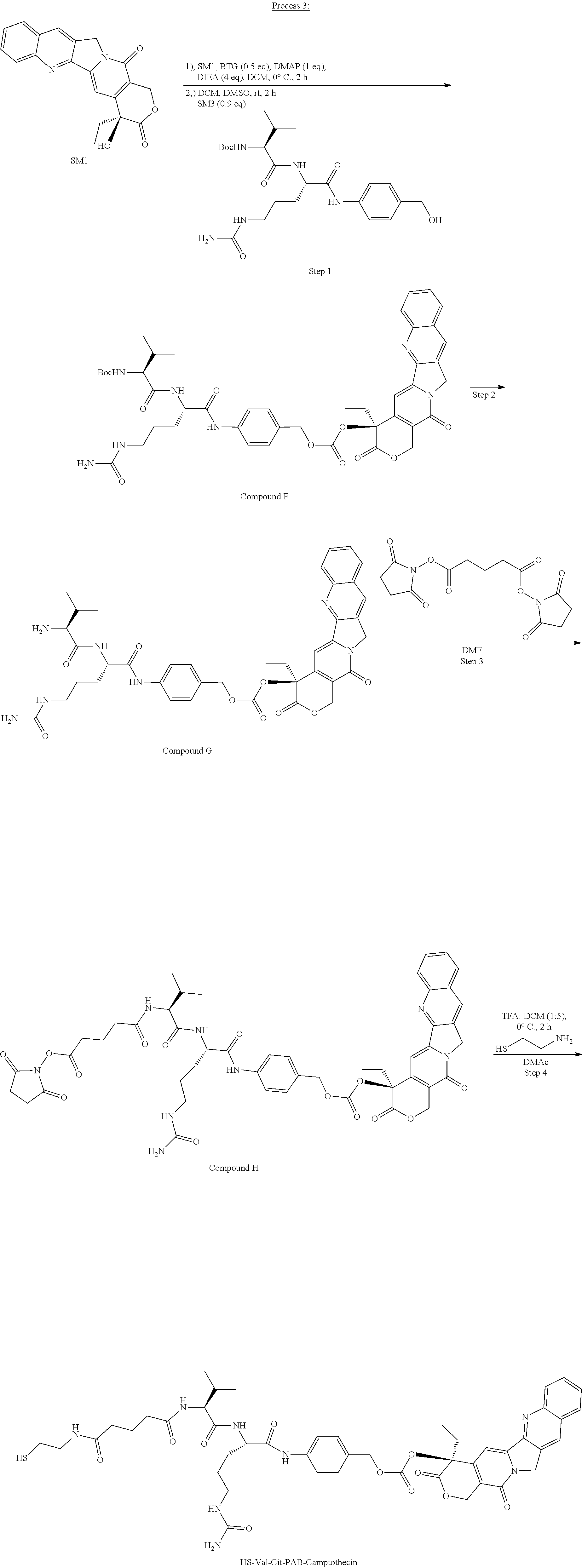

First, camptothecin was reacted with 0.5 equivalent of an agent that acts as a carbonyl source, 1 equivalent of 4-Dimethylaminopyridine (DMAP), and 4 equivalents of N,N-Diisopropylethylamine (DIPEA) in DCM at 0° C. for 2 hours. Next, the resulting compound was reacted with 0.9 equivalent of Boc-Val-Cit dipeptide in DCM and DMSO at room temperature for 2 hours. In step 2, Compound F was deprotected to form Compound G. In Step 3, Compound G was reacted with bis(2,5-dioxopyrrolidin-1-yl) glutarate in DMF to provide Compound H. In Step 4, Compound H was reacted with 2-aminoethane-1-thiol in trifluoroacetic acid (TFA) and DCM, in a 1:5 ratio, at 0° C., for 2 hours. Finally, the compound was purified to give the SH-VC-PAB-Camptothecin compound.

### Example 17: Synthesis of Doxorubicin with a Thiol-Containing Linker

This Example describes a synthesis of a payload, Doxorubicin, to a linker with a thiol.

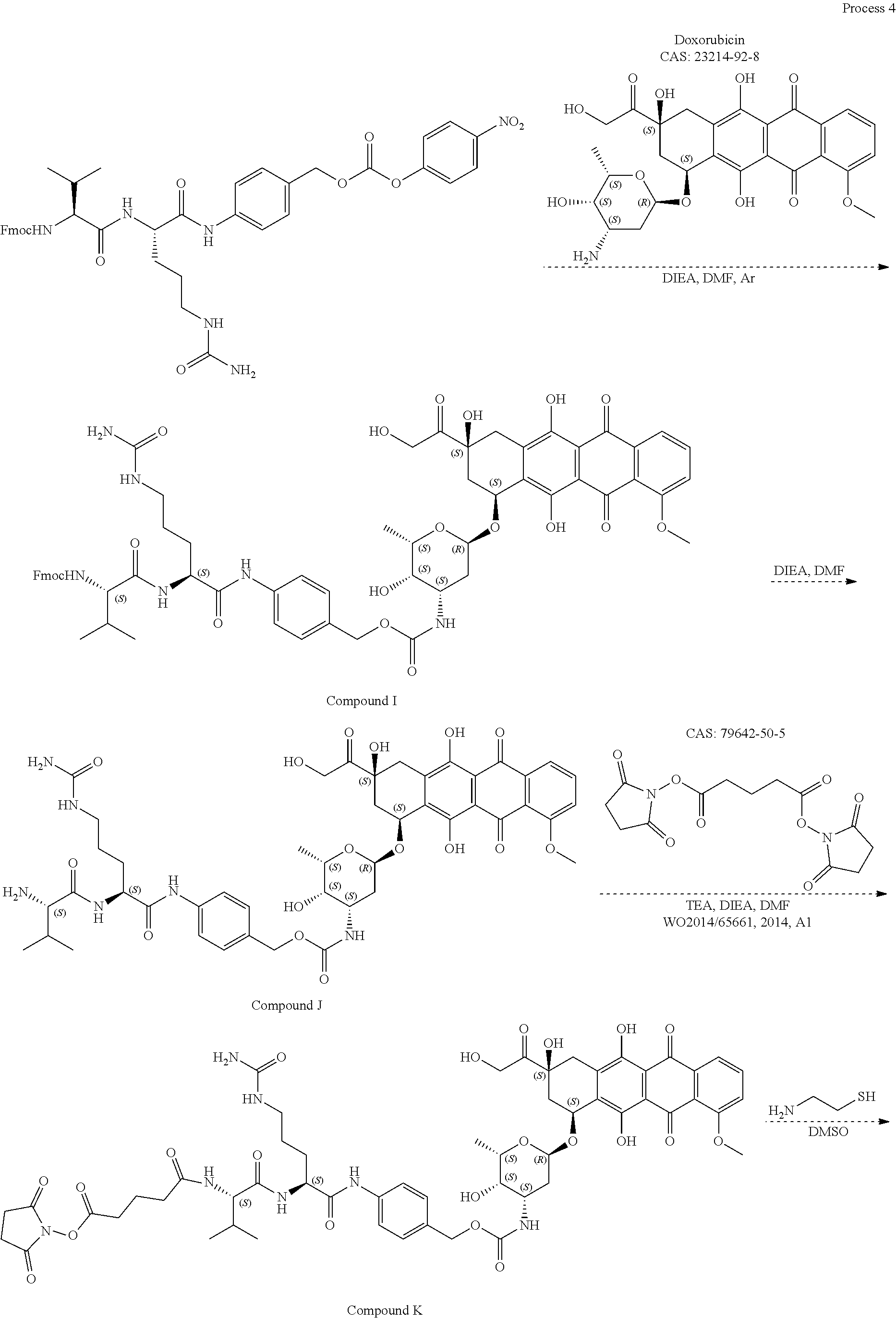

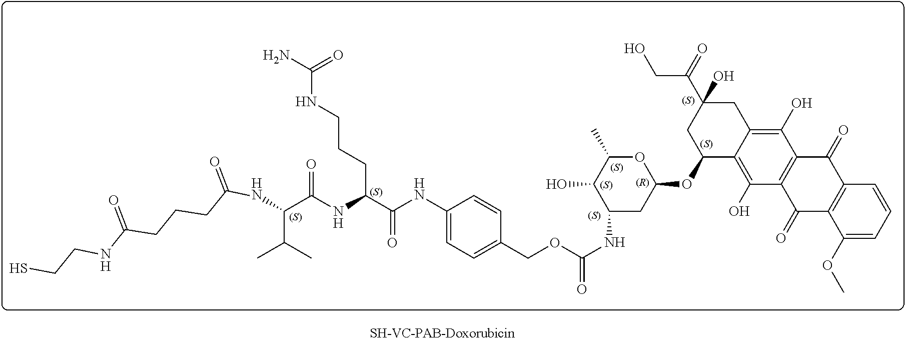

In Step 1, an Fmoc protected VC-PAB compound was reacted with doxorubicin in DIA and DMF to form Compound I. Next, Compound I was deprotected using DEA and DMF to form Compound J. Next, Compound J was reacted with bis(2,5-dioxopyrrolidin-1-yl) glutarate, TEA, DIEA, and DMA to provide Compound K. In the last step, Compound K was reacted with 2-aminoethane-1-thiol to provide the final compound, SH-VC-PAB-Doxorubicin.

The procedures disclosed herein can be conducted in a number of ways based on the teachings contained herein and synthetic procedures known in the art. In the description of the synthetic methods described above, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. It should be recognized that illustrated embodiments are only examples of the disclosed product and methods and should not be considered a limitation on the scope of the embodiments. Rather, the scope of the embodiments is defined by the claims. We therefore claim all that comes within the scope and spirit of these claims.

What is claimed is:

1. A conjugate of Formula A′, Formula B′, Formula C′, or Formula D′: 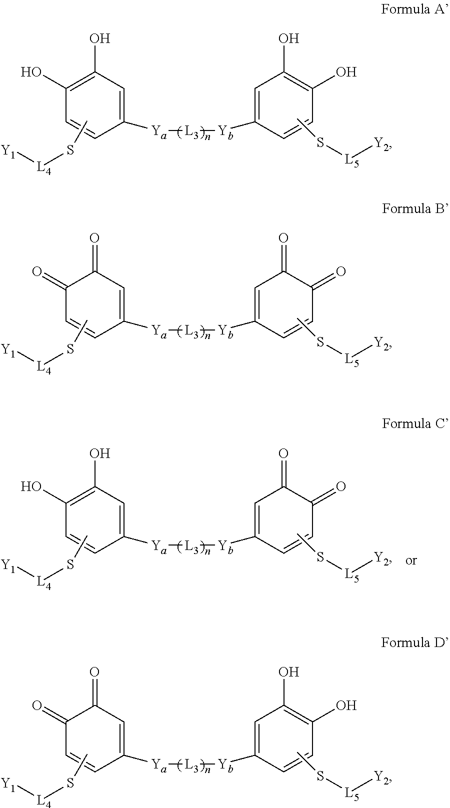
wherein:
Yₐ is a first polypeptide comprising a first terminal tag comprising (X₁)ₘ₁X₂X₃X₄X₅, wherein X₁-X₅ is any amino acid provided that at least one amino acid of X₁-X₅ comprises a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, wherein Yₐ is linked to “S” via the tyrosine or a portion thereof;
Y_{b} is a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ comprises a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof, wherein Y_{b} is linked to “S” via the tyrosine or a portion thereof;
n is an integer greater than 0;
L₃ is an optional third linker;
L₄ is an optional fourth linker;
L₅ is an optional fifth linker;
Y₁ is a first payload; and
Y₂ is a second payload.

2. The conjugate of claim 1, wherein the tyrosine or a portion thereof is selected from the group consisting of

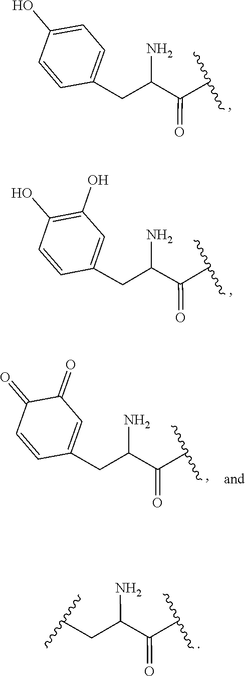 [SMILES: *C(=O)C(N)Cc1ccc(O)cc1 ; *C(=O)C(N)Cc1ccc(O)c(O)c1 ; *C(=O)C(N)CC1=CC(=O)C(=O)C=C1 ; *CC(N)C(*)=O]

3. The conjugate of claim 1-2, wherein the first terminal tag comprises at least two amino acids selected from the group consisting of aspartate (D), glutamate (E), arginine (R), and lysine (K).

4. The conjugate of any one of claims 1-3, wherein the second terminal tag comprises at least two amino acids selected from the group consisting of aspartate (D), glutamate (E), arginine (R), and lysine (K).

5. The conjugate of claim 1-2, wherein the first terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

6. The conjugate of any one of claims 1-5, wherein the second terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

7. The conjugate of any one of claims 1-6, wherein the first terminal tag comprises SGGY, SGY, SGGGY, ESY, EESY, ESSY, ESSSY, SESY, SGGGY, EESY, SSSY, SNNY, SSNY, EGGY, SESY, or SEGY.

8. The conjugate of any one of claims 1-6, wherein the second terminal tag comprises EEEY, EEEEY, SSSEY, SSEEY, SSSSY, or SSNNY.

9. The conjugate of any one of claims 1-7, wherein Yₐ, Y_{b′} or both Yₐ and Y_{b} are glycosylated.

10. The conjugate of any one of claims 1-9, wherein Yₐ, Y_{b′} or both Yₐ and Y_{b} comprise a non-terminal tyrosine.

11. The conjugate of any one of claims 1-10, wherein at least one of L₃, L₄, or L₅ is a cleavable linker.

12. The conjugate of claim 11, wherein the cleavable linker is an electrophilically cleavable linker, a nucleophilically cleavable linker, a photocleavable linker, a metal cleavable linker, an electrolytically-cleavable linker, an acid cleavable linker, or a proteolytically cleavable linker.

13. The conjugate of claim 12, wherein the cleavable linker further includes a pegylated group, a sugar group, or a modification that increases hydrophilicity.

14. The conjugate of claim 11, wherein the cleavable linker is cleavable under reductive and/or oxidative conditions.

15. The conjugate of claim 11, wherein the cleavable linker is cleavable under acidic conditions.

16. The conjugate of claim 11, wherein the cleavable linker comprises a disulfide bond.

17. The conjugate of claim 11, wherein the cleavable linker is a proteolytically cleavable linker and comprises a protease recognition sequence.

18. The conjugate of claim 17, wherein the protease recognition sequence is recognized by a protease selected from the group comprising a metalloprotease, cathepsin B, and tobacco etch virus (TEV).

19. The conjugate of claim 11, wherein the cleavable linker comprises a dipeptide, tripeptide or tetrapeptide

20. The conjugate of claim 19, wherein the dipeptide is a valine-citrulline (Val-Cit) dipeptide, a valine-lysine dipeptide, a valine-alanine dipeptide.

21. The conjugate of claim 19, wherein the tetrapeptide is a glycine-glycine-phenylalanine-glycine (GGFG) tetrapeptide.

22. The conjugate of claim 11, wherein the cleavable linker is selected from the group comprising PABC (p-aminobenzyl alcohol), glucuronide, and MABC (m-aminobenzyl alcohol).

23. The conjugate of claim 11 or 22, wherein the cleavable linker is Val-Cit-PABC.

24. The conjugate of claim 11, wherein “Y1-L4-S-” and/or “—S-L5-Y2” is selected from the group consisting of:

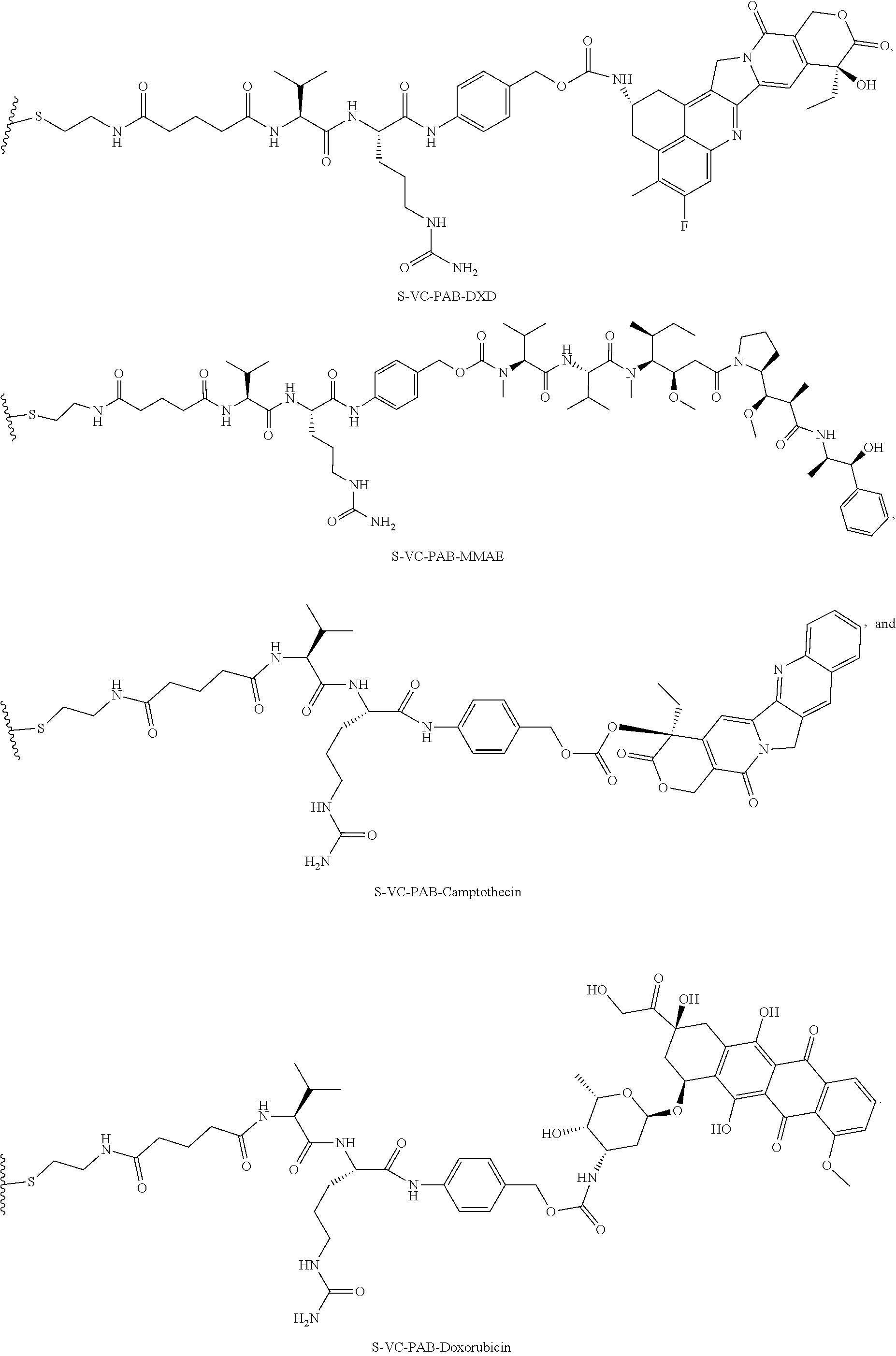

25. The conjugate of any one of claims 1-23, wherein Yₐ is a heavy chain variable region (VH), a light chain variable region (VL), a heavy chain, a light chain, a constant chain of an antibody or antibody fragment, a peptide, or a cyclic peptide.

26. The conjugate of claim 25, wherein the peptide is a binding peptide.

27. The conjugate of any one of claims 1-26, wherein Y_{b} is a heavy chain variable region (VH), a light chain variable region (VL), a heavy chain, a light chain, a constant chain of an antibody, antibody fragment, a peptide, or a cyclic peptide.

28. The conjugate of claim 27, wherein the peptide is a binding peptide.

29. The conjugate of any one of claims 1-28, wherein Yₐ and Y_{b} are attached via L₃.

30. The conjugate of any one of claims 1-28, wherein L₃ comprises a peptide sequence, a dimerization and docking domain, a leucine zipper, or knobs-into-holes.

31. The conjugate of any one of claims 1-30, wherein L₃ comprises a peptide bond, a disulfide bond, a maleimide bond, thioether bond, an azide-alkyne cycloaddition, a cystinyl-dopa, or a hydrogen bond.

32. The conjugate of any one of claims 1-28, wherein L₃ is a linker.

33. The conjugate of claim 32, wherein the linker comprises a sequence selected from the group consisting of (GS)ₙ₃, (G2S)ₙ₃, (G3S)ₙ₃, (G4S)ₙ₃, (G)ₙ₃, (GGSGGD)ₙ₃, (GGSGGE)ₙ₃, (GGGSGSGGGGS)ₙ₃, and (GGGGGPGGGGP)ₙ₃ and wherein n3 is an integer from 2 to 20.

34. The conjugate of any one of claims 1-33, wherein L₃ comprises a terminal tyrosine or a portion thereof.

35. The conjugate of any one of claims 1-34, wherein n is 1, 2, 3, 4, or 5.

36. The conjugate of any one of claims 1-34, wherein n is 1.

37. The conjugate of any one of claims 1-34 wherein Y₁ and Y₂ are the same.

38. The conjugate of any one of claims 1-34, wherein Y₁ and Y₂ are different.

39. The conjugate of any one of claims 1-38, wherein Y₁, Y₂, or both Y₁ and Y₂ is a small molecule.

40. The conjugate of claim 39, wherein the small molecule is selected from the group consisting of deruxtecan, exatecan, FL118, irinotecan, topotecan, SN-38, rubitecan, belotecan, lurototecan, gimatecan, diflomotecan, karenitecan, silatecan, namitecan, elomotecan, DRF-1042, delimotecan, NSC606985, chimmitecan, ZBH-1205, auristatin (MMAE, MMAF, MMAG, MMAH) calicheamicin, doxorubicin, taxol and taxol derivatives, maytansinoids (DM1-4), pyrolodiazepines (PBDs), tubulysins, eribulin, anthramycin, duocarmycin, anthracycline, and camptothecin (CPT), including the lactone and carboxylate forms of CPT.

41. The conjugate of claim 39, wherein the small molecule is selected from the group of a topoisomerase inhibitor and a tubulin inhibitor.

42. The conjugate of any one of claims 1-38, wherein Y₁ and Y₂, or both Y₁ and Y₂ comprises a nucleic acid, an immune agonist, a peptide, a cytokine, or a binding domain.

43. The conjugate of any one of claims 1-38, wherein Y₁ and Y₂, or both Y₁ and Y₂ comprises a nucleic acid.

44. The conjugate of claim 43, wherein Y₁ and Y₂, or both Y₁ and Y₂ comprises an oligonucleotide.

45. The conjugate of any one of claims 1-38, wherein Y₁ and Y₂, or both Y₁ and Y₂ comprises a peptide.

46. The conjugate of claim 45, wherein Y₁ and Y₂, or both Y₁ and Y₂ comprises a binding peptide.

47. A conjugate of Formula A-I, Formula B-I, Formula C-I, or Formula D-I: 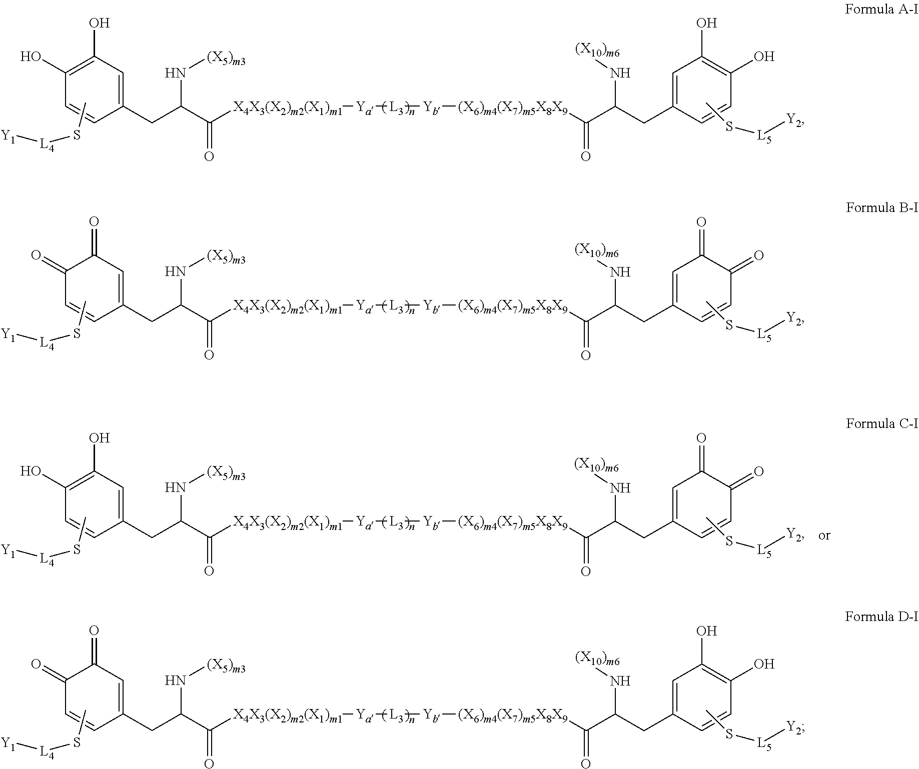
wherein:
Y_{a′} is a first polypeptide,
each of X₁, X₂, X₃, X₄, and X₅ is independently any amino acid, wherein m1, m2, and m3 are each an integer greater than or equal to 0;
Y_{b′} is a second polypeptide,
each of X₆, X₇, X₈, X₉, and X₁₀ is independently any amino acid, wherein m4, m5, and m6 are each an integer greater than or equal to 0, and wherein the —(X₁)ₘ₁(X₂)ₘ₂X₃X₄-moiety is different from the —(X₆)ₘ₁(X₇)ₘ₂X₈ X₉-moiety;
n is an integer greater than 0;
L₃ is an optional third linker;
L₄ is an optional fourth linker;
L₅ is an optional fifth linker;
Y₁ is a first payload; and
Y₂ is a second payload.

48. The conjugate of claim 47, wherein the —(X₁)ₘ₁(X₂)ₘ₂X₃X₄— moiety comprises at least two amino acids selected from the group consisting of aspartate (D), glutamate (E), arginine (R), and lysine (K).

49. The conjugate of any one of claims 47-48, wherein the —(X₆)ₘ₁(X₇)ₘ₂X₈ X₉-moiety comprises at least two amino acids selected from the group consisting of aspartate (D), glutamate (E), arginine (R), and lysine (K).

50. The conjugate of claim 47, wherein the —(X₁)ₘ₁(X₂)ₘ₂X₃X₄— moiety is selected from the group consisting of GGGG, RGGG, RGRG, RRRG, RRRR, EGGG, EGEG, EEEG, EEEE, GGGW, GGWG, RRRW, RRWR, EEEW, EEWE, DDDD, SGG, SG, KKKK, RRKK, RRRK, EDED, EDDD, EEDD, RRKK, KKGG, DDGG EES, ESSS, SSS, SNN, SSN, SEG, SSSE, EGG, SSEE, SES, ESS, ES, SR, SK, SN, ER, EK, EG, SE, SHRK, SARK, SPPE, SSEE, SEEE, and SSSS.

51. The conjugate of any one of claims 47-50, wherein the —(X₆)ₘ₁(X₇)ₘ₂X₈ X₉— moiety is selected from the group consisting of GGGG, RGGG, RGRG, RRRG, RRRR, EGGG, EGEG, EEEG, EEEE, GGGW, GGWG, RRRW, RRWR, EEEW, EEWE, DDDD, SGG, SG, KKKK, RRKK, RRRK, EDED, EDDD, EEDD, RRKK, KKGG, DDGG, EES, ESSS, SSS, SNN, SSN, SEG, SSSE, EGG, SSEE, SES, ESS, ES, SR, SK, SN, ER, EK, EG, SE, SHRK, SARK, SPPE, SSEE, SEEE, and SSSS.

52. The conjugate of any one of claims 47-51, wherein the —(X₁)ₘ₁(X₂)ₘ₂X₃X₄— moiety is SGG, SG, SGGG, ES, EES, ESS, ESSS, SES, SGGG, EES, SSS, SNN, SSN, EGG, SES, or SEG.

53. The conjugate of any one of claims 47-52, wherein the —(X₆)ₘ₁(X₇)ₘ₂X₈ X₉— moiety is EEE, EEEE, SSSE, SSEE, SSSS, or SSNN.

54. The conjugate of any one of claims 47-53, wherein Y_{a′}, Y_{b′}, or both Y_{a′} and Y_{b′} are glycosylated.

55. The conjugate of any one of claims 47-54, wherein at least one of L₃, L₄, or L₅ is a cleavable linker.

56. The conjugate of claim 55, wherein the cleavable linker is an electrophilically cleavable linker, a nucleophilically cleavable linker, a photocleavable linker, a metal cleavable linker, an electrolytically-cleavable linker, an acid cleavable linker, or a proteolytically cleavable linker.

57. The conjugate of claim 56, wherein the cleavable linker further include a pegylated group, a sugar group, or a modification that increases hydrophilicity.

58. The conjugate of claim 55, wherein the cleavable linker is cleavable under reductive and/or oxidative conditions.

59. The conjugate of claim 55, wherein the cleavable linker is cleavable under acidic conditions.

60. The conjugate of claim 55, wherein the cleavable linker comprises a disulfide bond.

61. The conjugate of claim 55, wherein the cleavable linker is a proteolytically cleavable linker and comprises a protease recognition sequence.

62. The conjugate of claim 6117, wherein the protease recognition sequence is recognized by a protease selected from the group comprising a metalloprotease, cathepsin B, and tobacco etch virus (TEV).

63. The conjugate of claim 55, wherein the cleavable linker comprises a dipeptide, tripeptide or tetrapeptide

64. The conjugate of claim 63, wherein the dipeptide is a valine-citrulline (Val-Cit) dipeptide, a valine-lysine dipeptide, a valine-alanine dieptide.

65. The conjugate of claim 63, wherein the tetrapeptide is a glycine-glycine-phenylalanine-glycine (GGFG) tetrapeptide.

66. The conjugate of claim 55, wherein the cleavable linker is selected from the group comprising PABC (p-aminobenzyl alcohol), glucuronide, and MABC (m-aminobenzyl alcohol),

67. The conjugate of any one of claims 47-66, wherein Y_{a′} is a heavy chain variable region (VH), a light chain variable region (VL), a heavy chain, a light chain, a constant chain of an antibody or antibody fragment, a peptide, or a cyclic peptide.

68. The conjugate of claim 67, wherein the peptide is a binding peptide.

69. The conjugate of any one of claims 47-67, wherein Y_{b′} is a heavy chain variable region (VH), a light chain variable region (VL), a heavy chain, a light chain, a constant chain of an antibody, antibody fragment, a peptide, or a cyclic peptide.

70. The conjugate of claim 69, wherein the peptide is a binding peptide.

71. The conjugate of any one of claims 47-70, wherein Yₐ, and Y_{b′} are attached via L₃.

72. The conjugate of any one of claims 47-71, wherein L₃ comprises a peptide sequence, a dimerization and docking domain, a leucine zipper, or knobs-into-holes.

73. The conjugate of any one of claims 47-72, wherein L₃ comprises a peptide bond, a disulfide bond, a maleimide bond, thioether bond, an azide-alkyne cycloaddition, a cystinyl-dopa, or a hydrogen bond.

74. The conjugate of any one of claims 47-71, wherein L₃ is a linker.

75. The conjugate of claim 74, wherein the linker comprises a sequence selected from the group consisting of (GS)ₙ₃, (G2S)ₙ₃, (G3S)ₙ₃, (G4S)ₙ₃, (G)ₙ₃, (GGSGGD)ₙ₃, (GGSGGE)ₙ₃, (GGGSGSGGGGS)ₙ₃, and (GGGGGPGGGGP)ₙ₃ and wherein n3 is an integer from 2 to 20.

76. The conjugate of any one of claims 47-75, wherein n is 1, 2, 3, 4, or 5.

77. The conjugate of any one of claims 47-76, wherein n is 1.

78. The conjugate of any one of claims 47-77, wherein Y₁ and Y₂ are the same.

79. The conjugate of any one of claims 47-77, wherein Y₁ and Y₂ are different.

80. The conjugate of any one of claims 47-79, wherein Y₁, Y₂, or both Y₁ and Y₂ is a small molecule.

81. The conjugate of claim 80, wherein the small molecule is selected from the group consisting of deruxtecan, exatecan, FL118, irinotecan, topotecan, SN-38, rubitecan, belotecan, lurototecan, gimatecan, diflomotecan, karenitecan, silatecan, namitecan, elomotecan, DRF-1042, delimotecan, NSC606985, chimmitecan, ZBH-1205, auristatin (MMAE, MMAF, MMAG, MMAH) calicheamicin, doxorubicin, taxol and taxol derivatives, maytansinoids (DM1-4), pyrolodiazepines (PBDs), tubulysins, eribulin, anthramycin, duocarmycin, anthracycline, and camptothecin (CPT), including the lactone and carboxylate forms of CPT.

82. The conjugate of claim 80, wherein the small molecule is selected from the group of a topoisomerase inhibitor and a tubulin inhibitor.

83. The conjugate of any one of claims 47-79, wherein Y₁ and Y₂, or both Y₁ and Y₂ comprises a nucleic acid, an immune agonist, a peptide, a cytokine, or a binding domain.

84. The conjugate of any one of claims 47-79, wherein Y₁ and Y₂, or both Y₁ and Y₂ comprises a nucleic acid.

85. The conjugate of claim 84, wherein Y₁ and Y₂, or both Y₁ and Y₂ comprises an oligonucleotide.

86. The conjugate of any one of claims 47-79, wherein Y₁ and Y₂, or both Y₁ and Y₂ comprises a peptide.

87. The conjugate of claim 86, wherein Y₁ and Y₂, or both Y₁ and Y₂ comprises a binding peptide.

88. A polypeptide comprising a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof; and a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof.

89. The polypeptide of claim 88, wherein the first terminal is on a N-terminus of the polypeptide and the second terminal tag is on the C-terminus of the polypeptide.

90. The polypeptide of claim 88 or 89, wherein the polypeptide comprises a non-terminal tyrosine.

91. The polypeptide of claim 90, wherein the polypeptide is modified to expose the non-terminal tyrosine to be accessible to an enzyme.

92. A composition of a first polypeptide and a second polypeptide, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine or a portion thereof, or X₁X₂X₃, wherein X₁-X₃ is any amino acid provided that at least one amino acid of X₁-X₃ is a tyrosine or a portion thereof; and wherein m1 is an integer greater than or equal to 0, and a second polypeptide comprising a second terminal tag different from the first terminal tag, the second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0.

93. The composition of claim 92, wherein the first polypeptide and/or the second polypeptide comprise a non-terminal tyrosine.

94. The composition of claim 93, wherein the first polypeptide and/or the second polypeptide is modified to expose the non-terminal tyrosine to be accessible to an enzyme.

95. The polypeptide or composition of claims 88-94, wherein the first terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

96. The polypeptide or composition of claims 88-95, wherein the second terminal tag comprises at least one of GGGGY, RGGGY, RGRGY, RRRGY, RRRRY, EGGGY, EGEGY, EEEGY, EEEEY, GGGWY, GGWGY, RRRWY, RRWRY, EEEWY, EEWEY, DDDDY, SGGY, SGY, KKKKY, RRKKY, RRRKY, EDEDY, EDDDY, EEDDY, RRKKY, KKGGY, DDGGY, EESY, ESSSY, SSSY, SNNY, SSNY, SEGY, SSSEY, EGGY, SSEEY, SESY, ESSY, ESY, SRY, SKY, SNY, ERY, EKY, EGY, SEYP, SHRKY, SARKY, SPPEY, SSEEY, SEEEY, and SSSSY.

97. The polypeptide or composition of claims 88-96, wherein the first terminal tag comprises SGGY, SGY, SGGGY, ESY, EESY, ESSY, ESSSY, SESY, SGGGY, EESY, SSSY, SNNY, SSNY, EGGY, SESY, or SEGY.

98. The polypeptide or composition of claims 88-97, wherein the second terminal tag comprises EEEY, EEEEY, SSEEY, SSSEY, SSSSY, or SSNNY.

99. A method of covalently linking at least two polypeptides to at least two payloads, the method comprising:
a) contacting a first polypeptide of the at least two polypeptides and a first payload of the at least two payloads using a first tyrosinase, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, and
b) contacting a second polypeptide of the at least two polypeptides and a second payload of the at least two payloads using a second tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof.

100. The method of claim 99, wherein the first tyrosinase, the second tyrosinase, or both the first tyrosinase and the second tyrosinase is Agricus bisporus tyrosinase (abTYR).

101. The method of any one of claims 99-100, wherein the first tyrosinase, the second tyrosinase, or both comprises a sequence at least 80% identity to any one of SEQ ID NOs: 1-6, 52, or 53.

102. The method of any one of claims 99-101, wherein the first tyrosinase, the second tyrosinase, or both the first tyrosinase and the second tyrosinase is a Catenase.

103. The method of claim 102, wherein the first tyrosinase and the second tyrosinase is a Catenase.

104. The method of any one of claims 99-102, wherein the first tyrosinase, the second tyrosinase, or both comprises a sequence at least 90% identity any one of SEQ ID NOs: 2-6.

105. The method of any one of claims 99-102, wherein the first tyrosinase, the second tyrosinase, or both comprises a sequence at least 90% identity to SEQ ID NO: 2.

106. The method of any one of claims 99-102, wherein the first tyrosinase is Agricus bisporus tyrosinase (abTYR) and the second tyrosinase is Catenase.

107. The method of any one of claims 99-106, wherein the first tyrosinase and the second tyrosinase are provided at the same time.

108. The method of any one of claims 99-106, wherein the first tyrosinase is provided first followed by the second tyrosinase.

109. A method of covalently linking at least two polypeptides to at least two payloads, the method comprising:
a) contacting a first polypeptide of the at least two polypeptides and a first payload of the at least two payloads in the presence of a tyrosinase, wherein the first polypeptide comprises a first terminal tag comprising (X₁)ₘ₁X₂X₃X₄X₅, wherein X₁-X₅ is any amino acid provided that at least one amino acid of X₁-X₅ is a tyrosine or a portion thereof, and wherein m1 is an integer greater than or equal to 0, or X₁X₂X₃, wherein X₁-X₃ is any amino acid provided that at least one amino acid of X₁-X₃ is a tyrosine or a portion thereof, and
b) contacting a second polypeptide of the at least two polypeptides and a second payload of the at least two payloads in the presence of the tyrosinase, wherein the second polypeptide comprises a second terminal tag comprising (X₆)ₘ₂X₇X₈X₉X₁₀, wherein X₆-X₁₀ is any amino acid provided that at least one amino acid of X₆-X₁₀ is a tyrosine or a portion thereof, and wherein m2 is an integer greater than or equal to 0, or X₁X₂X₃, wherein X₁-X₃ is any amino acid provided that at least one amino acid of X₁-X₃ is a tyrosine or a portion thereof.

110. The method of claim 109, wherein the first polypeptide is contacted with the tyrosinase at a temperature between 0-15° C.

111. The method of claim 109 or 110, wherein the first polypeptide is contacted with the tyrosinase at a temperature of 4° C.

112. The method of any one of claims 109-111, wherein the second polypeptide is contacted with the tyrosinase at a temperature between 25-50° C.

113. The method of any one of claims 109-112, wherein the second polypeptide is contacted with the tyrosinase at a temperature of 30° C.

114. The method of any one of claims 109-113, wherein the first polypeptide is contacted with the tyrosinase for 5-70 min.

115. The method of any one of claims 109-114, wherein the first polypeptide is contacted with the tyrosinase for 45 min.

116. The method of any one of claims 109-115, wherein the first polypeptide is contacted with the tyrosinase for 60 min.

117. The method of any one of claims 109-116, wherein the second polypeptide is contacted with the tyrosinase for more than 15 min.

118. The method of any one of claims 109-117, wherein the second polypeptide is contacted with the tyrosinase for 60-160 min.

119. The method of any one of claims 109-118, wherein the second polypeptide is contacted with the tyrosinase for 90 min.

120. The method of any one of claims 109-119, wherein the second polypeptide is contacted with the tyrosinase for 120 min.

121. The method of any one of claims 109-120, wherein the tyrosinase comprises at least 80% identity to any one of SEQ ID NOs: 1-6, 52, or 53.

122. A conjugate of Formula E′, Formula F′, Formula G′, Formula H′, Formula J′, or Formula K′: 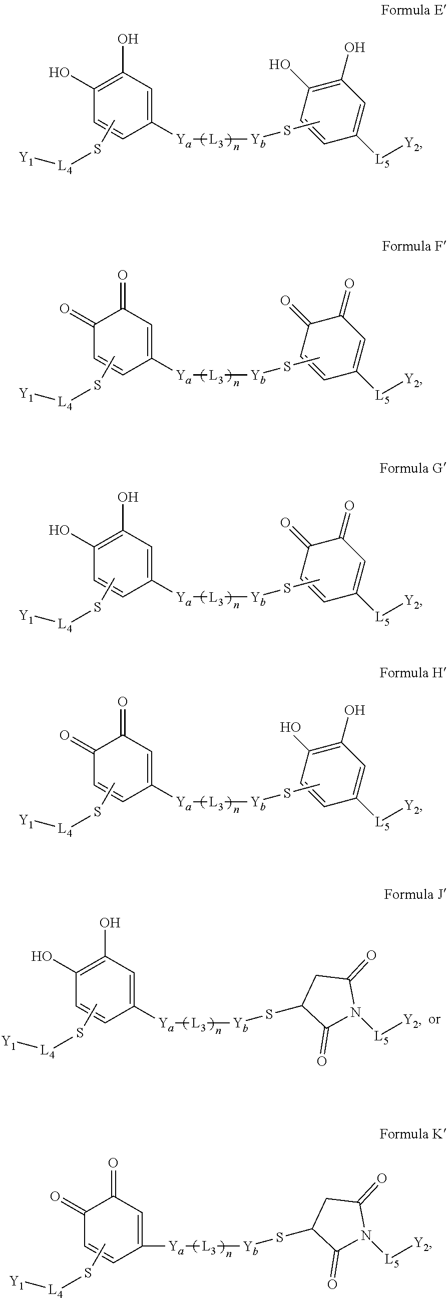
wherein:
Yₐ is a first polypeptide comprising a first terminal tag comprising (X₁)ₘ₁X₂X₃X₄X₅Y, wherein X₁-X₅ is any amino acid provided that no more than two amino acids of X₂, X₃, X₄, and X₅ are aspartate or glutamate and wherein m1 is an integer greater than or equal to 0;
Y_{b} is a second polypeptide;
n is an integer greater than 0;
L₃ is an optional third linker;
L₄ is an optional fourth linker;
L₅ is an optional fifth linker;
Y₁ is a first payload; and
Y₂ is a second payload.

123. A compound of formula L-I: 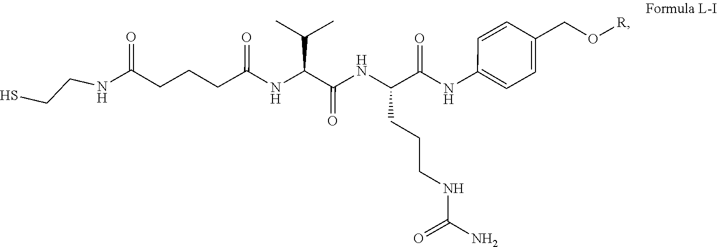
wherein R is a payload.

124. The compound of claim 123, wherein the compound is selected from the group consisting of:

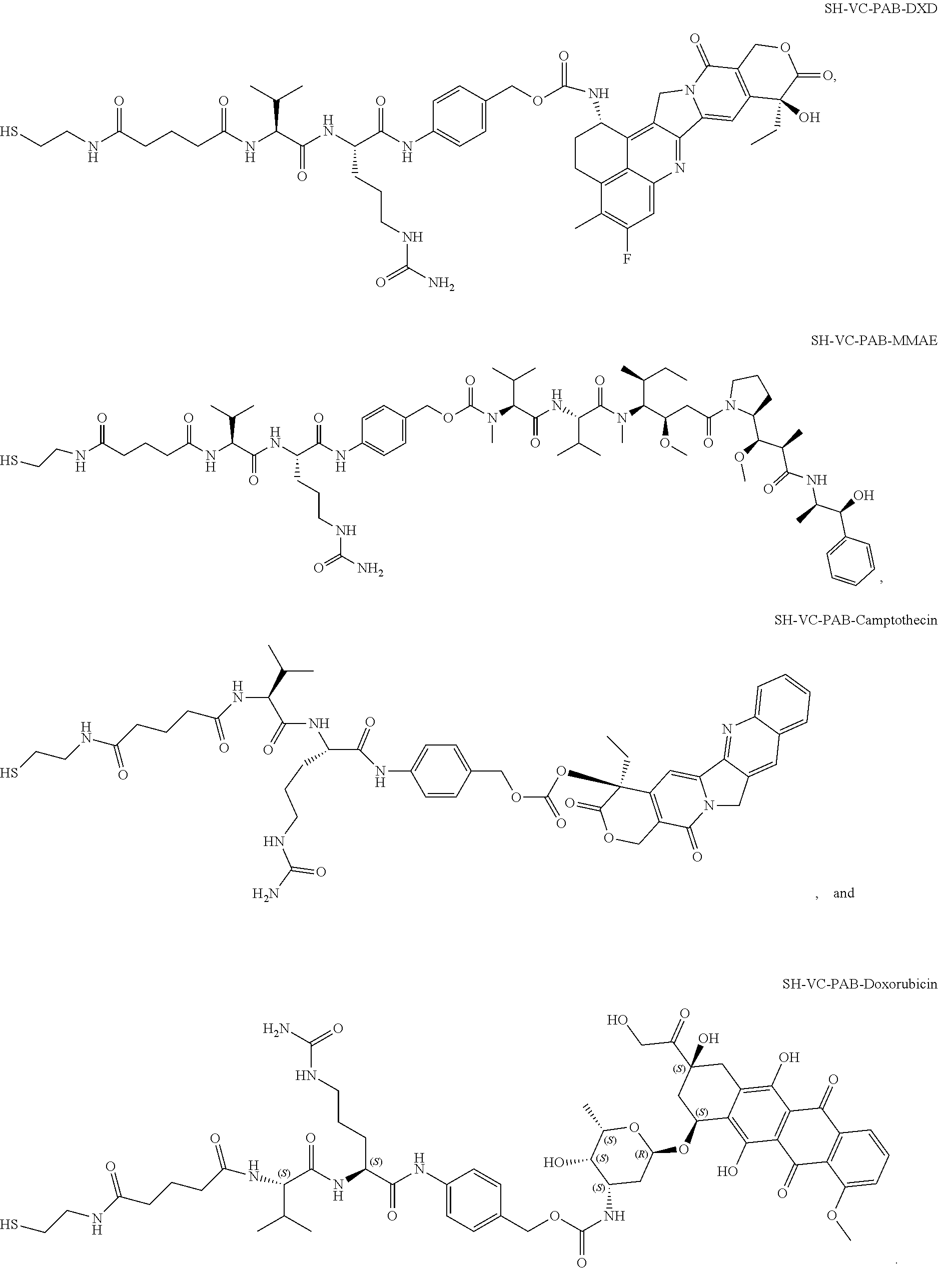

125. A polypeptide-conjugate represented by Formula S1, Formula S2, Formula S3, or Formula S4, 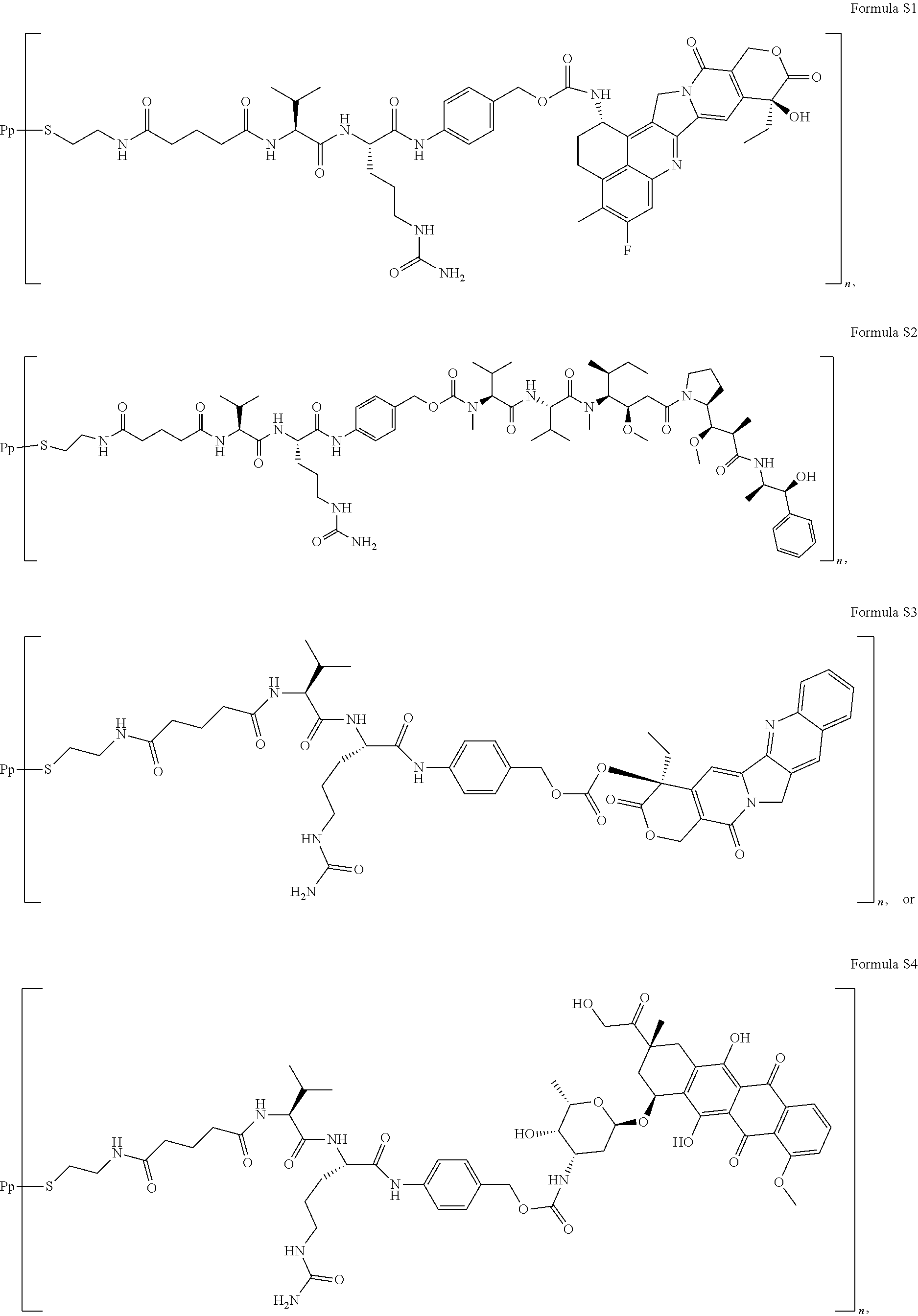
wherein: Pp is a polypeptide and contains an oxidized tyrosine;
n is 1, 2, or 3;
and wherein the “-S-” of the compound is conjugated to the oxidized tyrosine on the polypeptide.

126. The polypeptide-conjugate of claim 129, wherein the polypeptide is an enzyme, an antibody or a portion thereof, a structural polypeptide, a ligand for a receptor, or a receptor.

127. The polypeptide-conjugate of claim 129 or 130, wherein the polypeptide is an antibody or a portion thereof.

128. The polypeptide-conjugate of any one of claims 129-131, wherein the polypeptide is a heavy chain variable region (VH), a light chain variable region (VL), a heavy chain, a light chain, a constant chain of an antibody or antibody fragment, a peptide, or a cyclic peptide.


SEQUENCE LISTING (113 sequences)

SEQ ID NO: 1 - protein, 576 aa, Agaricus bisporus
MSDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHNTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYGFVTTQTENPALRLLSSFQRAKSDHETQYALYDWVIHATFRYYELNNSFSIIFYFDEGEGCTLESIIGTVDAFRGTTSENCANCARSQDLIAEGFVHLNYYIGCDIGQHADHEDDAVPLYEPTRVKEYLKKRKIGCKVVSAEGELTSLVVEIKGAPYYLPVGEARPKLDHEKPIVILDDIIHRVN

SEQ ID NO: 2 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 3 - protein, 305 aa, synthetic construct
MSNKYRVRKNVLHLTDTEKRDFVRTVLILKEKGIYDRYIAWHGAAGKFHTPPGSRRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 4 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMEVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 5 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 6 - protein, 305 aa, synthetic construct
MSNKSRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 7 - protein, 5 aa, synthetic construct
GGGGY

SEQ ID NO: 8 - protein, 5 aa, synthetic construct
RGGGY

SEQ ID NO: 9 - protein, 5 aa, synthetic construct
RGRGY

SEQ ID NO: 10 - protein, 5 aa, synthetic construct
RRRGY

SEQ ID NO: 11 - protein, 5 aa, synthetic construct
RRRRY

SEQ ID NO: 12 - protein, 5 aa, synthetic construct
EGGGY

SEQ ID NO: 13 - protein, 5 aa, synthetic construct
EGEGY

SEQ ID NO: 14 - protein, 5 aa, synthetic construct
EEEGY

SEQ ID NO: 15 - protein, 5 aa, synthetic construct
EEEEY

SEQ ID NO: 16 - protein, 5 aa, synthetic construct
GGGWY

SEQ ID NO: 17 - protein, 5 aa, synthetic construct
GGWGY

SEQ ID NO: 18 - protein, 5 aa, synthetic construct
RRRWY

SEQ ID NO: 19 - protein, 5 aa, synthetic construct
RRWRY

SEQ ID NO: 20 - protein, 5 aa, synthetic construct
EEEWY

SEQ ID NO: 21 - protein, 5 aa, synthetic construct
EEWEY

SEQ ID NO: 22 - protein, 5 aa, synthetic construct
DDDDY

SEQ ID NO: 23 - protein, 4 aa, synthetic construct
SGGY

SEQ ID NO: 24 - protein, 5 aa, synthetic construct
KKKKY

SEQ ID NO: 25 - protein, 5 aa, synthetic construct
RRKKY

SEQ ID NO: 26 - protein, 5 aa, synthetic construct
RRRKY

SEQ ID NO: 27 - protein, 5 aa, synthetic construct
EDEDY

SEQ ID NO: 28 - protein, 5 aa, synthetic construct
EDDDY

SEQ ID NO: 29 - protein, 5 aa, synthetic construct
EEDDY

SEQ ID NO: 30 - protein, 5 aa, synthetic construct
RRKKY

SEQ ID NO: 31 - protein, 5 aa, synthetic construct
KKGGY

SEQ ID NO: 32 - protein, 5 aa, synthetic construct
DDGGY

SEQ ID NO: 33 - protein, 4 aa, synthetic construct
EEEY

SEQ ID NO: 34 - protein, 5 aa, synthetic construct
HHHHH

SEQ ID NO: 35 - protein, 6 aa, synthetic construct
HHHHHH

SEQ ID NO: 36 - protein, 10 aa, synthetic construct
EQKLISEEDL

SEQ ID NO: 37 - protein, 8 aa, synthetic construct
DYKDDDDK

SEQ ID NO: 38 - protein, 8 aa, synthetic construct
WSHPQFEK

SEQ ID NO: 39 - protein, 9 aa, synthetic construct
YPYDVPDYA

SEQ ID NO: 40 - protein, 5 aa, synthetic construct
RYIRS

SEQ ID NO: 41 - protein, 4 aa, synthetic construct
FHHT

SEQ ID NO: 42 - protein, 17 aa, synthetic construct
WEAAAREACCRECCARA

SEQ ID NO: 43 - protein, 4 aa, synthetic construct
EGGY

SEQ ID NO: 44 - protein, 5 aa, synthetic construct
SSEEY

SEQ ID NO: 45 - protein, 4 aa, synthetic construct
ESSY

SEQ ID NO: 46 - protein, 5 aa, synthetic construct
SHRKY

SEQ ID NO: 47 - protein, 5 aa, synthetic construct
SARKY

SEQ ID NO: 48 - protein, 5 aa, synthetic construct
SPPEY

SEQ ID NO: 49 - protein, 4 aa, synthetic construct
SESY

SEQ ID NO: 50 - protein, 5 aa, synthetic construct
SEEEY

SEQ ID NO: 51 - protein, 5 aa, synthetic construct
SSSSY

SEQ ID NO: 52 - protein, 382 aa, Agaricus bisporus
SLLATVGPTGGVKNRLDIVDFVRDEKFFTLYIRALQAIQDKDQSDYSSFFQLSGIHGLPFTPWAKPKDTPTVPYESGYCTHSQVLFPTWHRVYVSIYEQILQEAAKGIAKKFTVHKKEWAQAAEDLRQPYWDTGFALVPPDEIIKLEQVKITNYDGTKITVRNPILRYSFHPIDPSFNGYPNFDTWKTTVRNPDADKKENIPALIGKLDLEADSTREKTYNMLKENANWEAFSNHGEFDDTHANSLEAVHDDIHGFVGRGAIRGHMTHALFAAFDPIFWLHHSNVDRHLSLWQALYPGVWVTQGPEREGSMGFAPGTELNKDSALEPFYETEDKPWTSVPLTDTALLNYSYPDFDKVKGGTPDLVRDYINDHIDRRYGIKKS

SEQ ID NO: 53 - protein, 389 aa, Agaricus bisporus
SDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTENMLTKNYTWELFSNHGAWGAHANSLEMVHNTVHFLIGRDPTLDPLVPGHMGSVPHAAFDPIFWMHHCNVDRLLALWQTMNYDVYVSEGMNREATMGUPGQVLTEDSPLEPFYTKNQDPWQSDDLEDWETLGFSYPDFDPVKGKSKEEKSVYINDWVHKHYG

SEQ ID NO: 54 - protein, 4 aa, synthetic construct
EESY

SEQ ID NO: 55 - protein, 5 aa, synthetic construct
ESSSY

SEQ ID NO: 56 - protein, 4 aa, synthetic construct
SSSY

SEQ ID NO: 57 - protein, 4 aa, synthetic construct
SNNY

SEQ ID NO: 58 - protein, 4 aa, synthetic construct
SSNY

SEQ ID NO: 59 - protein, 4 aa, synthetic construct
SEGY

SEQ ID NO: 60 - protein, 5 aa, synthetic construct
SSSEY

SEQ ID NO: 61 - protein, 4 aa, synthetic construct
SEYP

SEQ ID NO: 62 - protein, 5 aa, synthetic construct
SGGGY

SEQ ID NO: 63 - protein, 5 aa, synthetic construct
SSNNY

SEQ ID NO: 64 - protein, 4 aa, synthetic construct
GGFG

SEQ ID NO: 65 - protein, 40 aa, synthetic construct
  VARIANT 5..40: note = GS repeats may be deleted
GSGSGSGSGSGSGSGSGSGSGSGSGSGSGSGSGSGSGSGS

SEQ ID NO: 66 - protein, 60 aa, synthetic construct
  VARIANT 7..60: note = GGS repeats may be deleted
GGSGGSGGSGGSGGSGGSGGSGGSGGSGGSGGSGGSGGSGGSGGSGGSGGSGGSGGSGGS

SEQ ID NO: 67 - protein, 80 aa, synthetic construct
  VARIANT 9..80: note = GGGS repeats may be deleted
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 68 - protein, 100 aa, synthetic construct
  VARIANT 11..100: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 69 - protein, 20 aa, synthetic construct
  VARIANT 3..20: note = Residues may be deleted
GGGGGGGGGGGGGGGGGGGG

SEQ ID NO: 70 - protein, 120 aa, synthetic construct
  VARIANT 13..120: note = GGSGGD repeats may be deleted
GGSGGDGGSGGDGGSGGDGGSGGDGGSGGDGGSGGDGGSGGDGGSGGDGGSGGDGGSGGDGGSGGDGGSGGDGGSGGDGGSGGDGGSGGDGGSGGDGGSGGDGGSGGDGGSGGDGGSGGD

SEQ ID NO: 71 - protein, 120 aa, synthetic construct
  VARIANT 13..120: note = GGSGGE repeats may be deleted
GGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGE

SEQ ID NO: 72 - protein, 220 aa, synthetic construct
  VARIANT 23..220: note = GGGSGSGGGGS repeats may be deleted
GGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGSGGGSGSGGGGS

SEQ ID NO: 73 - protein, 220 aa, synthetic construct
  VARIANT 23..220: note = GGGGGPGGGGP repeats may be deleted
GGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGPGGGGGPGGGGP

SEQ ID NO: 74 - protein, 4 aa, synthetic construct
GGGG

SEQ ID NO: 75 - protein, 4 aa, synthetic construct
RGGG

SEQ ID NO: 76 - protein, 4 aa, synthetic construct
RGRG

SEQ ID NO: 77 - protein, 4 aa, synthetic construct
RRRG

SEQ ID NO: 78 - protein, 4 aa, synthetic construct
RRRR

SEQ ID NO: 79 - protein, 4 aa, synthetic construct
EGGG

SEQ ID NO: 80 - protein, 4 aa, synthetic construct
EGEG

SEQ ID NO: 81 - protein, 4 aa, synthetic construct
EEEG

SEQ ID NO: 82 - protein, 4 aa, synthetic construct
EEEE

SEQ ID NO: 83 - protein, 4 aa, synthetic construct
GGGW

SEQ ID NO: 84 - protein, 4 aa, synthetic construct
GGWG

SEQ ID NO: 85 - protein, 4 aa, synthetic construct
RRRW

SEQ ID NO: 86 - protein, 4 aa, synthetic construct
RRWR

SEQ ID NO: 87 - protein, 4 aa, synthetic construct
EEEW

SEQ ID NO: 88 - protein, 4 aa, synthetic construct
EEWE

SEQ ID NO: 89 - protein, 4 aa, synthetic construct
DDDD

SEQ ID NO: 90 - protein, 4 aa, synthetic construct
KKKK

SEQ ID NO: 91 - protein, 4 aa, synthetic construct
RRKK

SEQ ID NO: 92 - protein, 4 aa, synthetic construct
RRRK

SEQ ID NO: 93 - protein, 4 aa, synthetic construct
EDED

SEQ ID NO: 94 - protein, 4 aa, synthetic construct
EDDD

SEQ ID NO: 95 - protein, 4 aa, synthetic construct
EEDD

SEQ ID NO: 96 - protein, 4 aa, synthetic construct
KKGG

SEQ ID NO: 97 - protein, 4 aa, synthetic construct
DDGG

SEQ ID NO: 98 - protein, 4 aa, synthetic construct
ESSS

SEQ ID NO: 99 - protein, 4 aa, synthetic construct
SSSE

SEQ ID NO: 100 - protein, 4 aa, synthetic construct
SSEE

SEQ ID NO: 101 - protein, 4 aa, synthetic construct
SHRK

SEQ ID NO: 102 - protein, 4 aa, synthetic construct
SARK

SEQ ID NO: 103 - protein, 4 aa, synthetic construct
SPPE

SEQ ID NO: 104 - protein, 4 aa, synthetic construct
SEEE

SEQ ID NO: 105 - protein, 4 aa, synthetic construct
SSSS

SEQ ID NO: 106 - protein, 4 aa, synthetic construct
SGGG

SEQ ID NO: 107 - protein, 4 aa, synthetic construct
SSNN

SEQ ID NO: 108 - protein, 36 aa, synthetic construct
  VARIANT 13..36: note = GGSGGD repeats may be deleted
GGSGGDGGSGGDGGSGGDGGSGGDGGSGGDGGSGGD

SEQ ID NO: 109 - protein, 36 aa, synthetic construct
  VARIANT 13..36: note = GGSGGE repeats may be deleted
GGSGGEGGSGGEGGSGGEGGSGGEGGSGGEGGSGGE

SEQ ID NO: 110 - protein, 33 aa, synthetic construct
  VARIANT 12..33: note = GGGSGSGGGGS repeats may be deleted
GGGSGSGGGGSGGGSGSGGGGSGGGSGSGGGGS

SEQ ID NO: 111 - protein, 33 aa, synthetic construct
  VARIANT 12..33: note = GGGGGPGGGGP repeats may be deleted
GGGGGPGGGGPGGGGGPGGGGPGGGGGPGGGGP

SEQ ID NO: 112 - protein, 6 aa, synthetic construct
GGGGCY

SEQ ID NO: 113 - protein, 6 aa, synthetic construct
SSEEEY